SEQUENCE LISTING (21 sequences)

SEQ ID NO: 1 - DNA, 20 bp, synthetic construct
  source 1..20: mol_type = other DNA
gagaagggccgaggtattgt

SEQ ID NO: 2 - DNA, 20 bp, synthetic construct
  source 1..20: mol_type = other DNA
acaatacctcggcccttctc

SEQ ID NO: 3 - DNA, 20 bp, synthetic construct
  source 1..20: mol_type = other DNA
acactacctcggccctcctc

SEQ ID NO: 4 - DNA, 20 bp, synthetic construct
  source 1..20: mol_type = other DNA
acactacctcagccctcctc

SEQ ID NO: 5 - DNA, 20 bp, synthetic construct
  source 1..20: mol_type = other DNA
acgaaggtctgtagcaactc

SEQ ID NO: 6 - DNA, 20 bp, synthetic construct
  source 1..20: mol_type = other DNA
gagttgctacagaccttcgt

SEQ ID NO: 7 - DNA, 20 bp, synthetic construct
  source 1..20: mol_type = other DNA
gagttactacagaccatcgt

SEQ ID NO: 8 - DNA, 20 bp, synthetic construct
  source 1..20: mol_type = other DNA
acactacctcagccctcctc

SEQ ID NO: 9 - DNA, 20 bp, synthetic construct
  source 1..20: mol_type = other DNA
tctgtgatcgtgtaggatgc

SEQ ID NO: 10 - DNA, 20 bp, synthetic construct
  source 1..20: mol_type = other DNA
gcatcctacacgatcacaga

SEQ ID NO: 11 - DNA, 20 bp, synthetic construct
  source 1..20: mol_type = other DNA
gcaccctacacgatcaaaga

SEQ ID NO: 12 - DNA, 20 bp, synthetic construct
  source 1..20: mol_type = other DNA
gcaccctacatgatcaaaga

SEQ ID NO: 13 - DNA, 20 bp, synthetic construct
  source 1..20: mol_type = other DNA
ggttgttgacgagtagtgtg

SEQ ID NO: 14 - DNA, 20 bp, synthetic construct
  source 1..20: mol_type = other DNA
cacactactcgtcaacaacc

SEQ ID NO: 15 - DNA, 20 bp, synthetic construct
  source 1..20: mol_type = other DNA
cacattactcgtcatcaacc

SEQ ID NO: 16 - DNA, 20 bp, synthetic construct
  source 1..20: mol_type = other DNA
cacattactagtcatcaacc

SEQ ID NO: 17 - DNA, 20 bp, synthetic construct
  source 1..20: mol_type = other DNA
tggtgtgtacttgtgcagag

SEQ ID NO: 18 - DNA, 20 bp, synthetic construct
  source 1..20: mol_type = other DNA
ctctgcacaagtacacacca

SEQ ID NO: 19 - DNA, 20 bp, synthetic construct
  source 1..20: mol_type = other DNA
ctctacacaagtacatacca

SEQ ID NO: 20 - DNA, 20 bp, synthetic construct
  source 1..20: mol_type = other DNA
ctctacacaattacatacca

SEQ ID NO: 21 - DNA, 20 bp, synthetic construct
  source 1..20: mol_type = other DNA
gagttactatagaccatcgt

(19) United States
(12) Patent Application Publication
(10) Pub. No.: US 2025/0354986 A1
(43) Pub. Date: Nov. 20, 2025

(54) AFFINITY ENCODED OSCILLATOR ARRAYS, METHODS, AND RELATED ASPECTS FOR MEASURING MOLECULAR BINDING KINETICS

(71) Applicant: ARIZONA BOARD OF REGENTS ON BEHALF OF ARIZONA STATE UNIVERSITY, Scottsdale, AZ (US)

(72) Inventors: Shaopeng Wang, Chandler, AZ (US); Shuo-Hui Cao, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 19/213,763

(22) Filed: May 20, 2025

Related U.S. Application Data
(60) Provisional application No. 63/649,551, filed on May 20, 2024.

(51) Int. Cl. G01N 33/543; G01N 21/552

(52) U.S. Cl. CPC G01N 33/54373; G01N 21/553; G01N 33/54313; G01N 2201/122; G01N 2333/035; G01N 2333/726

(57) ABSTRACT

Provided herein are methods of performing multiplex detection of ligand binding kinetics. In some embodiments, the methods include contacting ligands with an array of nucleic acid barcoded oscillators disposed on a first surface of a substrate that comprises an electrically conductive coating, applying an AC electric field to the substrate sufficient to induce the nucleic acid barcoded oscillators to oscillate proximal to the first surface of the substrate, and detecting changes in oscillation amplitudes of the nucleic acid barcoded oscillators over a duration to produce sets of ligand binding data. In some embodiments, the methods also include contacting barcode decoding nucleic acids with the array of nucleic acid barcoded oscillators applying an AC electric field to the substrate sufficient to induce the nucleic acid barcoded oscillators to oscillate proximal to the first surface of the substrate, and detecting changes in oscillation amplitudes of the nucleic acid barcoded oscillators over a duration to produce sets of barcode decoding data.

### CROSS-REFERENCE TO RELATED APPLICATIONS

This application claims priority to, and the benefit of, U.S. Provisional Patent Application Ser. No. 63/649,551, filed May 20, 2024, the disclosure of which is incorporated herein by reference.

### STATEMENT OF GOVERNMENT SUPPORT

This invention was made with government support under R33 CA235294 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on May 20, 2025, is named 0391_0106_SL.xml and is 19,381 bytes in size.

### BACKGROUND

Array-based detection techniques have made remarkable progress in recent years and it has been demonstrated its capacity in rapid and high-throughput screening of biochemical molecules. With increasing demands in biological and biomedical applications towards low-cost and high-throughput measurements, traditional array detections with spot size around hundreds of micrometers have exposed its limitations such as high sample consumption, extensive incubation time, and limited array density. As a result, nanoarrays, with their much smaller dimensions, have garnered significant attention. Although the top-down lithographic nanofabrication techniques (such as dip-pen nanolithography, electron beam lithography, or nanoimprint lithography) and the bottom-up self-assembly nanopattern techniques (like DNA origami) have enabled the generation of ordered array patterns with biomolecular spots nearing 100 nm, laborious fabrication processes still cannot bridge the gap between design and the practical application. In addition, it is challenging to develop simple, sensitive, and effective readout approaches toward the ultra-miniaturization array formats. Although a few detection methods have been reported, these restrictions still prevent further development of these approaches. For instance, fluorescence-based detection involves complicated fluorophore labeling, which not only has risk to affect intrinsic bio-interactions, but suffers signal stability issues caused by photobleaching. The scanning probe microscopy based high-resolution detection, such as Kelvin probe force microscopy or atomic force microscopy, requires relatively long scanning time and carries the risk of biomolecular degradation due to tip-sample interactions.

Benefiting from the highly sensitive measurement capacity of the surface plasmon resonance (SPR) effect, optical detection of individual plasmonic nanoparticles, typically ranging from tens to hundreds of nanometers in size, has been achieved. Theoretically, each nanoparticle could function as a sensing unit. As a result, a simple and cost-effective fabrication strategy has been proposed to construct nanoarrays through directly depositing biofunctionalized nanoparticles onto the substrate, with each single nanoparticle serving as an individual array spot. For example, combined with optical dark-field spectroscopy, others have demonstrated that the immobilized single gold nanorod could give plasmonic wavelength shift responding to bound proteins. However, when it comes to multiplexing detection, different from lithography-fabricated arrays with geometric order to encode each spot through its predefined position, it is difficult to distinguish the randomly distributed nanoparticles with various biofunctionalizations if there are no effective encoding-decoding design at single nanoparticle resolution. An ingenious solution has been proposed, wherein one type of biofunctionalized nanoparticle is adsorbed onto the chip at a time, and multiple rounds of adsorption are performed. This enables the identification of nanoparticle types through continuously recording their adsorption positions during array fabrication. But the slow batch by batch adsorption for array fabrication and detection may reduce the efficiency in high-throughput screening of large biochemical libraries, thereby limiting its applications in practice.

Based on the SPR microscopy, we have reported the nano-oscillator technique to image nanoparticles tethered on the gold substrate, in which the charged nanoparticle can be driven into oscillation by an external alternating electric field, and the oscillation amplitude obtained by the plasmonic image is proportional to the net charge of the nanoparticle. Different from mass sensitive label-free techniques that the signal is limited by the molecular weight of analytes, charge distribution will be altered in most biomolecular interactions, including small molecules interactions. Through detecting the charge changes in this process, the interaction between biomolecules is effectively measured. Based on this strategy, the phosphorylation of peptides and the molecular binding events have been measured. In principle, every single nanoparticle with specific biomolecule conjugated could be served as an entity in a nanoarray for high-throughput detection, if they can be individually addressed.

Accordingly, there is a need for effective multiplexing techniques for measuring binding kinetics of molecular ligands, including drug candidates, with membrane proteins as well as other types of biomolecular binding partners.

### SUMMARY

This disclosure describes oscillator arrays, systems, kits, computer readable media, and related methods for determining binding kinetics of ligands with membrane proteins and other types of biomolecules. In some embodiments, for example, the present disclosure provides a nanoarray platform that involves the assembly of DNA barcoded nano-oscillators onto a sensor surface in a single step, resulting in the formation of a random nanoarray. To address individual nano-oscillators in the array, an integrated and encoding-decoding strategy using DNA sequence specific binding affinities is implemented in some embodiments. As an example demonstration, herpes simplex virus-1 (HSV-1) displayed G protein-coupled receptors (GPCRs) were studied. GPCRs are the largest membrane protein family in humans (>800 members), GPCRs play crucial roles in various cellular processes and are also popular drug targets, but they are notoriously hard to study due to the difficulty in extracting and the fragility in preserving their functional conformations. Avoiding the extraction and purification of membrane proteins, virion display technology keeps GPCRs in their native lipid membrane microenvironment. In some aspects of the present disclosure, various GPCRs-displayed virions are utilized to functionalize nano-oscillators, and binding kinetics and affinities of small molecule ligands are characterized through the charge-sensitive nano-oscillator array. In some embodiments, sequence specific hybridization affinity is used to address GPCRs in individual nano-oscillators. The nanoarray sensors of the present disclosure provide a simple and multiplexed detection tool in high-throughput screening for biological, biochemical, and medical applications. These and other aspects will be apparent upon a complete review of the present disclosure, including the accompanying figures.

In one aspect, the present disclosure provides a method of performing multiplex detection of ligand (e.g., an antibody, a small molecule, or the like) binding kinetics. The method includes sequentially screening multiple target ligands using an array of nucleic acid barcoded oscillators disposed on a first surface of a substrate that comprises an electrically conductive coating, applying an alternating current electric field to the substrate sufficient to induce the nucleic acid barcoded oscillators to oscillate proximal to the first surface of the substrate, and detecting changes, if any, in oscillation amplitudes of the nucleic acid barcoded oscillators over a duration to produce sets of ligand binding data. The method also includes decoding the nucleic acid barcodes of the nucleic acid barcoded oscillators by contacting barcode decoding nucleic acids with the array of nucleic acid barcoded oscillators disposed on the first surface of the substrate, applying the alternating current electric field to the substrate sufficient to induce the nucleic acid barcoded oscillators to oscillate proximal to the first surface of the substrate, and detecting changes, if any, in oscillation amplitudes of the nucleic acid barcoded oscillators over a duration to produce sets of barcode decoding data. In some embodiments, in lieu of sequentially screening multiple target ligands, those target ligands can be screened in parallel together using the same array of nucleic acid barcoded oscillators (e.g., as long as the target ligands do not have cross-talk with one another).

In another aspect, the present disclosure provides a method of performing multiplex detection of ligand (e.g., an antibody, a small molecule, or the like) binding kinetics. The method includes (a) contacting a first ligand with an array of nucleic acid barcoded oscillators disposed on a first surface of a substrate that comprises an electrically conductive coating. The nucleic acid barcoded oscillators each comprise a nanoparticle attached to the first surface via one or more linker moieties in which at least a first nucleic acid barcoded oscillator comprises one or more first ligand binding moieties and one or more first barcode coding nucleic acids attached to the nanoparticle of the first nucleic acid barcoded oscillator and in which at least a second nucleic acid barcoded oscillator comprises one or more second ligand binding moieties and one or more second barcode coding nucleic acids attached to the nanoparticle of the second nucleic acid barcoded oscillator. The first ligand binding moieties differ from the second ligand binding moieties. The first barcode coding nucleic acids differ from the second barcode coding nucleic acids. In addition, the first ligand is contacted with the array of nucleic acid barcoded oscillators under conditions sufficient for the first ligand to at least partially bind to the first and/or second ligand binding moieties. The method also includes (b) applying an alternating current electric field to the substrate sufficient to induce the nucleic acid barcoded oscillators to oscillate proximal to the first surface of the substrate, and (c) detecting changes, if any, in oscillation amplitudes of at least the first and second nucleic acid barcoded oscillators over a duration to produce a first set of ligand binding data. The method also includes (d) removing the first ligand from the array of nucleic acid barcoded oscillators, and (e) repeating steps (a)-(c) using a second ligand that differs from the first ligand to produce a second set of ligand binding data, wherein the second ligand is contacted with the array of nucleic acid barcoded oscillators under conditions sufficient for the second ligand to at least partially bind to the first and/or second ligand binding moieties. The method also includes (f) contacting one or more first barcode decoding nucleic acids with the array of nucleic acid barcoded oscillators disposed on the first surface of the substrate, wherein the first barcode decoding nucleic acids are at least partially complementary to the first and/or second barcode coding nucleic acids, and wherein the first barcode decoding nucleic acids are contacted with the array of nucleic acid barcoded oscillators under conditions sufficient for the first barcode decoding nucleic acids to at least partially hybridize with the first and/or second barcode coding nucleic acids, (g) applying the alternating current electric field to the substrate sufficient to induce the nucleic acid barcoded oscillators to oscillate proximal to the first surface of the substrate, and (h) detecting changes, if any, in oscillation amplitudes of at least the first and second nucleic acid barcoded oscillators over a duration to produce a first set of barcode decoding data. In addition, the method also includes (i) removing the first barcode decoding nucleic acids from the array of nucleic acid barcoded oscillators, and (j) repeating steps (f)-(h) using one or more second barcode decoding nucleic acids to produce a second set of barcode decoding data, wherein the second barcode decoding nucleic acids are at least partially complementary to the first and/or second barcode coding nucleic acids, wherein the second barcode decoding nucleic acids differ from the first barcode decoding nucleic acids, and wherein the second barcode decoding nucleic acids are contacted with the array of nucleic acid barcoded oscillators under conditions sufficient for the second barcode decoding nucleic acids to at least partially hybridize with the first and/or second barcode coding nucleic acids, thereby performing the multiplex detection of the ligand binding kinetics.

In some embodiments, step (d) comprises washing the first ligand from the array of nucleic acid barcoded oscillators using a buffer. In some embodiments, the method includes detecting changes, if any, in oscillation amplitudes of at least the first and second nucleic acid barcoded oscillators over a duration to produce an additional set of ligand binding data prior performing step (e). In some embodiments, step (i) comprises washing the first barcode decoding nucleic acids from the array of nucleic acid barcoded oscillators using a buffer. In some embodiments, the method includes detecting changes, if any, in oscillation amplitudes of at least the first and second nucleic acid barcoded oscillators over a duration to produce an additional set of barcode decoding data prior performing step (j).

In some embodiments, the method includes using the first and/or second ligand in a pharmaceutical agent development process based at least in part on the first and/or second set of ligand binding data and the first and/or second set of barcode decoding data. In some embodiments, the method includes administering the first and/or second ligand to a subject in need thereof based at least in part on the first and/or second set of ligand binding data and the first and/or second set of barcode decoding data.

In some embodiments, the method includes detecting the changes in the oscillation amplitudes of the first and second nucleic acid barcoded oscillators using a plasmonic imaging technique and/or a microscopic imaging technique. In some embodiments, the method includes quantifying the binding kinetics and binding affinity of the first and second ligands using the detected changes in the oscillation amplitudes of the first and second nucleic acid barcoded oscillators over the duration. In some embodiments, the method includes detecting the changes in the oscillation amplitudes of the first and second nucleic acid barcoded oscillators over the duration using a CMOS camera. In some embodiments, the method includes removing the second ligand from the array of nucleic acid barcoded oscillators prior to performing step (f). In some embodiments, step (d) comprises washing the first ligand from the array of nucleic acid barcoded oscillators.

In some embodiments, the electrically conductive coating comprises gold (Au), indium tin oxide (ITO), silver (Ag), copper (Cu), and/or aluminum (Al). In some embodiments, the linker moieties comprise polyethylene glycol (PEG) moieties and/or biomolecules.

In some embodiments, the first ligand binding moieties comprise at least a first protein that binds, or is capable of binding, to the first and/or second ligand, wherein the second ligand binding moieties comprise at least a second protein that binds, or is capable of binding, to the first and/or second ligand, and wherein the first and second proteins differ from one another. In some embodiments, virions are attached to the nanoparticles of the first and the second nucleic acid barcoded oscillators and wherein viral envelopes of the virions display the first or second proteins. In some embodiments, the virions comprise human herpes simplex virus-1 (HSV-1) virions. In some embodiments, the first and second proteins comprise different G-protein-coupled receptors (GPCRs).

In some embodiments, the nanoparticle comprises a metal nanoparticle (MNP). In some embodiments, the nanoparticle comprises a magnetic bead, a polystyrene nanoparticle, or a silica nanoparticle. In some embodiments, the first and/or second ligand binding moieties comprise proteins or nucleic acids.

In some embodiments, the detecting steps (c) and (h) comprises introducing an incident light toward a second surface of the substrate to induce a plasmonic wave at least proximal to the first surface of the substrate and detecting a change in intensity of the incident light reflected at an interface of the first surface of the substrate. In some embodiments, the method includes introducing the incident light via at least one objective lens and/or at least one prism. In some embodiments, the method includes introducing the incident light using a superluminescent diode (SLED).

In some embodiments, a first group of barcode coding nucleic acids comprises the first barcode coding nucleic acids and wherein a second group of barcode coding nucleic acids comprises the second barcode coding nucleic acids, wherein the first group comprises member nucleic acids having 5, 4, 3, 2, 1, or no non-complementary nucleotides with the first barcode decoding nucleic acid, and wherein the second group comprises member nucleic acids having 5, 4, 3, 2, 1, or no non-complementary nucleotides with the second barcode decoding nucleic acid. In some embodiments, the first surface of the substrate comprises one or more oscillators that lack a barcode coding nucleic acid attached to the nanoparticle. In some embodiments, one or more of the barcode coding nucleic acids and/or one or more of the barcode decoding nucleic acids comprise a sequence of nucleotides selected from the group consisting of: SEQ ID NOS: 1-20.

In another aspect, the present disclosure provides a method of producing an oscillator array device. The method includes forming an array of nucleic acid barcoded oscillators disposed on a first surface of a substrate that comprises an electrically conductive coating. The nucleic acid barcoded oscillators each comprise a nanoparticle attached to the first surface via one or more linker moieties in which at least a first nucleic acid barcoded oscillator comprises one or more first ligand binding moieties and one or more first barcode coding nucleic acids attached to the nanoparticle of the first nucleic acid barcoded oscillator and in which at least a second nucleic acid barcoded oscillator comprises one or more second ligand binding moieties and one or more second barcode coding nucleic acids attached to the nanoparticle of the second nucleic acid barcoded oscillator. The first ligand binding moieties differ from the second ligand binding moieties. In addition, the first barcode coding nucleic acids differ from the second barcode coding nucleic acids. In some embodiments, the method includes randomly forming the array of nucleic acid barcoded oscillators disposed on the first surface of the substrate.

In another aspect, the present disclosure provides an oscillator array device, comprising a substrate that comprises a first surface that comprises an electrically conductive coating and an array of nucleic acid barcoded oscillators disposed on the first surface of the substrate. The nucleic acid barcoded oscillators each comprise a nanoparticle attached to the first surface via one or more linker moieties in which at least a first nucleic acid barcoded oscillator comprises one or more first ligand binding moieties and one or more first barcode coding nucleic acids attached to the nanoparticle of the first nucleic acid barcoded oscillator and in which at least a second nucleic acid barcoded oscillator comprises one or more second ligand binding moieties and one or more second barcode coding nucleic acids attached to the nanoparticle of the second nucleic acid barcoded oscillator. The first ligand binding moieties differ from the second ligand binding moieties. In addition, the first barcode coding nucleic acids differ from the second barcode coding nucleic acids.

In some embodiments, the electrically conductive coating comprises gold (Au), indium tin oxide (ITO), silver (Ag), copper (Cu), and/or aluminum (Al). In some embodiments, the linker moieties comprise polyethylene glycol (PEG) moieties and/or biomolecules. In some embodiments, the oscillator array device further includes one or more spacer moieties attached to the first surface and/or to the linker moieties. In some embodiments, the oscillator array device further includes one or more blocking moieties attached to the first surface.

In some embodiments, the first ligand binding moieties comprise at least a first protein that binds, or is capable of binding, to a first and/or a second ligand, wherein the second ligand binding moieties comprise at least a second protein that binds, or is capable of binding, to the first and/or second ligand, wherein the first and second proteins differ from one another, wherein virions are attached to the nanoparticles of the first and the second nucleic acid barcoded oscillators, and wherein viral envelopes of the virions display the first or second proteins. In some embodiments, the virions comprise human herpes simplex virus-1 (HSV-1) virions. In some embodiments, the first and second proteins comprise different G-protein-coupled receptors (GPCRs). In some embodiments, a kit includes the oscillator array device.

In another aspect, the present disclosure provides a system for performing multiplex detection of ligand binding kinetics. The system includes a substrate having a first surface and a second surface opposite the first surface. The first surface comprises an electrically conductive coating. An array of nucleic acid barcoded oscillators is disposed on the first surface in which the nucleic acid barcoded oscillators each comprise a nanoparticle attached to the first surface via one or more linker moieties, in which at least a first nucleic acid barcoded oscillator comprises one or more first ligand binding moieties and one or more first barcode coding nucleic acids attached to the nanoparticle of the first nucleic acid barcoded oscillator, and in which at least a second nucleic acid barcoded oscillator comprises one or more second ligand binding moieties and one or more second barcode coding nucleic acids attached to the nanoparticle of the second nucleic acid barcoded oscillator. The first ligand binding moieties differ from the second ligand binding moieties. The first barcode coding nucleic acids differ from the second barcode coding nucleic acids. The system also includes a power source electrically connected to the substrate, which power source is configured to apply an alternating current electric field to the substrate, an objective lens or a prism disposed proximal to the second surface of the substrate, a light source configured to introduce light through the objective lens or the prism to induce a plasmonic wave at least proximal to the first surface of the substrate, and a detector configured to collect light reflected from the substrate. In addition, the system also includes a controller operably connected at least to the power source, the light source, and the detector, wherein the controller comprises, or is capable of accessing, computer readable media comprising non-transitory computer-executable instructions which, when executed by at least one electronic processor, perform at least: applying an alternating current electric field to the substrate to induce the nucleic acid barcoded oscillators to oscillate proximal to the first surface of the substrate using the power source; introducing an incident light toward the second surface of the substrate from the light source to induce the plasmonic wave at least proximal to the first surface of the substrate; and detecting changes in oscillation amplitudes of the first and second nucleic acid barcoded oscillators over a duration.

In another aspect, the present disclosure provides a computer readable media comprising non-transitory computer executable instruction which, when executed by at least electronic processor, perform at least: applying an alternating current electric field to a substrate having a first surface and a second surface opposite the first surface, wherein the first surface comprises an electrically conductive coating and an array of nucleic acid barcoded oscillators attached to the first surface via one or more linker moieties, and wherein the alternating current electric field induces the nucleic acid barcoded oscillators to oscillate proximal to the first surface of the substrate; introducing an incident light toward the second surface of the substrate from a light source to induce the plasmonic wave at least proximal to the first surface of the substrate; and detecting changes in oscillation amplitudes of the first and second nucleic acid barcoded oscillators over a duration.

### BRIEF DESCRIPTION OF DRAWINGS

FIG. 1A is a flow chart that schematically shows exemplary method steps of determining binding kinetics of a ligand according to some aspects disclosed herein.

FIG. 1B is a flow chart that schematically shows exemplary method steps of determining binding kinetics of a ligand according to some aspects disclosed herein.

FIG. 2 schematically shows a virion nanoarray for oscillation measurement.

FIG. 3 is a schematic diagram of an exemplary system suitable for use with certain aspects disclosed herein.

FIGS. 4A-4C: (a) Schematic illustration of the prism based SPR imaging setup for oscillation measurement, where WE, RE, and CE stand for the working, reference, and counter electrodes, respectively. Inset: assembly schematic of the nano-oscillator, where biotin-avidin interactions mediate virion-AuNP conjugation, DNA barcode loading, and PEG linker-based AuNP capture. (b) Typical SEM image of the nanocomplex oscillators, each of which was composed by one AuNP and one virion. The scale bar was 500 nm. (c) Typical snapshots of plasmonic images of individual nanocomplex oscillators under negative (top) and positive (bottom) potentials.

FIG. 5: Schematic illustration of the nano-oscillator sensor platform for molecular interaction screening and affinity-based decoding. The system utilizes L distinct affinity barcodes paired with M barcode groups, allowing the encoding of N virions via LM unique combinations. Each decoding cycle detects L affinity values, with full sensor decoding achieved through M sequential hybridizations.

FIGS. 6A and 6B: (a) Plasmonic image intensity histogram of 106 individual virion-AuNP nanocomplexes, presenting an averaged intensity of 170.6±31.6 mDeg for a single object. (b) Fourier spectra of a nano-oscillator showing oscillation amplitudes with (lighter grayscale) and without (darker grayscale) applying the alternating electric field. Insets show oscillation amplitudes as a function of time under the electric field. The frequency of the alternating electric field was 5 Hz.

FIGS. 7A-7H: (a) Plasmonic image of individual virion-AuNP nano-oscillators, where the virions display GPCR membrane proteins NTSR1 in their envelopes. (b) Distribution of the equilibrium dissociation constant (KD) for NTSR1-Neurotensin pair simultaneously measured by 35 individual nano-oscillators, presenting an averaged KD of 12.8±2.3 nM. The solid line is the Gaussian fitting to the distribution. (c-h) Binding curves (lighter grayscale) and the corresponding fitting curves (darker grayscale) of individual nano-oscillators marked in (a) responding to ligands, with the injection of ligands starting at 0 s, and the injection of buffer starting at 300 s.

FIGS. 8A and 8B: (a-b) Distribution of equilibrium affinity constant (K_{A}) measured by individual nano-oscillators with DNA barcodes hybridized with the corresponding DNA decoding sequences. The solid lines are the Gaussian fittings to the distributions. For each probe, 37 individual nano-oscillators were measured for statistics. Two DNA barcode groups used for decoding were tested. For the first group (a), the averaged K_{A} were (8.1±1.1)×10⁸ M⁻¹, (5.3±0.74)×10⁷ M⁻¹, and (5.9±0.82)×10⁶ M⁻¹, respectively for the barcodes complementary to, two bases mismatched to, and three bases mismatched to the corresponding DNA decoding sequence. For the second group (b), the averaged K_{A} were (7.1±0.98)×10⁸ M⁻¹, (6.5±1.1)×10⁷ M⁻¹, and (2.0±0.78)×10⁶ M⁻¹, respectively for the barcodes complementary to, two bases mismatched to, and three bases mismatched to the corresponding DNA decoding sequence.

FIGS. 9A-9G: (a) Plasmonic image of GPCRs displayed oscillator nanoarray. Nano-oscillators conjugated with the different type of virions are marked by stars with different colors (6 oscillators were selected for each type) according to the decoding result. (b-g) Binding curves (gray) of individual nano-oscillators marked in (a), including five specific GPCRs-ligands pairs of NTSR1-Neurotensin (b), OPRM1-DynorphinA (c), ADRB2-Propranolol (d), DRD1-D1 antagonist (e), and SSTR2-SRIF14 (f), and one K082 virion with no GPCR displayed on the envelope as a negative control to interact with the ligand (propranolol as an example) (g). The injection of ligands started at 0 s, and the injection of buffer started at 300 s. The averaged binding curves of the 6 individuals for each type of nano-oscillators were used to generate the fitting curves (except the case in (g) where the averaged curve was present, showing the not obvious responding) drawn by the corresponding colors in (a). The darker and lighter dots in the insets of (b-g) respectively indicated the KA values measured by the selected nano-oscillators to experience the sequential hybridizations with the two decoding sequences.

FIGS. 10A and 10B: SPR Images tracking the fabrication of nano-oscillators on gold film. (a) A time sequence of SPR images illustrating nano-oscillators' formation on the surface. The color map represents SPR image intensity in millidegrees (mDeg). (b) SPR intensity shifts over time at specific regions (indicated by numbers) where individual nano-oscillators adsorb onto the gold surface.

FIGS. 11A-11C: Statistical analysis of SPR intensity distributions of different nanoparticles on gold film. (a) Virons, (b) AuNPs, and (c) gold nanoparticle-viron complexes. Insets display typical SPR images of individual particles. The solid lines represent Gaussian fittings of the distributions.

FIGS. 12A-12D: Scanning electron microscopy images of nanocomplex oscillators. Arrows indicate the presence of AuNPs and virions. Scale bar: 200 nm.

FIG. 13: Absorbance spectra illustrating the distinct signatures of AuNPs, AuNP-virions, and DNA codes-AuNP-virions. The peaks at 600 nm are caused by the resonance of 150 nm AuNPs, while the increased signals at 280 nm (lightest grayscale curve) can be attributed to the protein features of virions attached to the AuNPs. Additionally, the increased signals around 260 nm (medium grayscale curve) demonstrate the successful labeling of DNA codes.

FIGS. 14A and 14B: Control experiments using (a) the K082 virion based oscillator with no GPCR displayed on the envelope and (b) the gold nanoparticle oscillator with no virion to test their responses to target ligands of Neurotensin.

FIG. 15: Plasmonic image of high-density ensemble virion oscillators on a gold chip, with the region containing ensemble virions outlined by the yellow dotted line. The virions were connected with gold film through thiol-PEG-NHS. Scale bar: 10 mm.

FIGS. 16A and 16B: (a) ensemble virion oscillators and (b) single oscillator responses to target ligands in dose-dependent measurements through oscillation technique, where the solid lines are global fitting of the data to the first order kinetic.

FIGS. 17A-17F: Barcode readout tests in single nano-oscillator with one kind of barcode sequence modified modification, where solid lines are fitting according to the first order kinetic model. Barcodes containing (a) 0-, (b) 2-, and (c) 3-nucleotide mismatches to the decoding sequence 1 and (d-f) barcodes containing (d) 0-, (e) 2-, and (f) 3-nucleotide mismatches to the decoding sequence 2 are separately used.

FIGS. 18A-18D: Spotting-free nano-oscillator array (SFNOA) for measuring ligand binding kinetics of membrane proteins in their native membrane environment. a) A nano-oscillator comprised of a HSV-1 virion displaying human GPCRs conjugated to a DNA barcoded gold nanoparticle and tethered to the sensing chip via flexible PEG linkers; b) Scanning electron microscopy image of a nano-oscillator; c) SFNOA showing intermixed nano-oscillators of different GPCRs on a plasmonic imaging system with electrical field driven oscillation; d) Molecular binding to the nano-oscillator conjugate changes the charge profile of the oscillator and consequently the oscillation amplitude, which allows accurate determination of the binding kinetics.

FIGS. 19A-19D: Measuring virion oscillation amplitude with plasmonic imaging. a) Plasmonic image of single virion oscillators, each showing a parabolic pattern (arising from the scattering of surface plasmonic waves by the virion). b) Snapshots of one oscillator during different phases of an oscillation cycle, where the image intensity change reflects the change in virion-substrate distance (and thus the oscillation amplitude). c) Virion-substrate distance (darker grayscale) of the oscillator in (b) and applied field (lighter grayscale) with frequency of 5 Hz. d) Fast Fourier Transform (FFT) of oscillation amplitude (lighter grayscale) showing a pronounced peak at 5 Hz, and the peak amplitude is the oscillation amplitude, where the black line is from the control obtained by performing FFT without applied electric field.

FIG. 20: Workflow of SFNOA. Step I: sequentially introduce each ligand to measure ligand binding kinetics to all individual oscillators; Step II: decode the array by sequentially measuring the hybridization affinities of all individual oscillators to each decoding sequence. The example schematic here shows two decoding groups. Each decoding group contains 3 barcodes (3+1 states). This combination can encode 42=16 different types of membrane proteins. Additional decoding groups can be added to expand the total encoding state to 4m, where m is the number of decoding groups.

FIGS. 21A-21D: Preliminary data of binding affinities of a group of DNA barcodes. a-c) Representative single nano-oscillator response curves for the decoding sequence binding to the DNA barcodes listed in Table 1, with averaged equilibrium affinity constant KA values listed in the graph. d) Histograms of KA of the 3 DNA barcodes hybridized with the DNA decoding sequence, measured by individual nano-oscillators. The solid lines are the Gaussian fittings to the distributions. For each type of barcode, 37 individual nano-oscillators were measured for statistics.

FIG. 22: Schematically shows the production of 315 human non-odorant VirD GPCRs.

FIGS. 23A-23D: Measuring small molecule binding to VirD-GPCR oscillators. a-c) Binding kinetic curves (oscillation amplitude changes vs. time) of three ligand/VirD GPCR pairs, where the solid lines are global fitting of the data to the first order kinetics. Each binding curve is average of at least 5 virions. Ligand structure, kinetic and equilibrium constants are labeled next to the graphs. d) Negative control experiment performed on virions without GPCRs displayed shows no binding to any of the small molecule ligands. Applied voltage: 0.4 V at 5 Hz. Buffer: diluted PBS buffer at pH 7.4.

FIGS. 24A-24D: SFNOA with affinity encoded multi-state DNA barcoding for multiplexed binding kinetics measurement. a) Plasmonic imaging of the oscillation amplitude of the SFNOA, with the barcode identified specific types of individual oscillators marked with the initial letters of the GPCR: ADRB2, DRD1, and K082. b)-d) Small molecule ligand binding response curves plotted in gray color for individual nano-oscillators marked in a, with the ligand/receptor names and fitted kinetic constants listed on the graph. The injection of ligands started at 0 s, and switched to buffer at 300 s. The averaged binding curves of the 6 individual nano-oscillators for each type of virions were used to generate the fitting curves (solid lines) in b and c. The colored dots in the insets of b-d are the corresponding DNA barcode affinity values of the selected nano-oscillators to the decoding sequence.

FIGS. 25A and 25B: High resolution PSM imaging of nanoparticle. a) Principle and setup of a prism coupled PSM; b) PSM image of a 100 nm polystyrene nanoparticle.

FIG. 26: Block diagram of the high throughput SFNOA system based upon PSM detection. A flow cell with optical window and electrodes is mounted on top of the sensor. A flow control system is implemented for precision sample delivery. Thermal control encompasses the prism and flow cell regions for reproducible and stable performance. Various control circuits and boards are implemented to support a commercially viable PSM detection system.

FIG. 27: Anti-V5 Western blot analysis to assess expression of GPCRs on purified virions used in this study. Note: For complete gel and blot data, see uncropped images in FIG. 32.

FIGS. 28A-28C: Fabrication of DNA-encoded gold nanoparticle-virion complexes for nano-oscillator construction. (a) Schematic illustration of the process to obtain a single batch of encoded complexes, each involving one kind of virion. (b) Schematic diagram showing the process for generating a library for nano-oscillator assembly. (c) Schematic illustration of the self-assembly of nanocomplexes on a PEG-modified gold sensor chip to form the nano-oscillator structure.

FIGS. 29A and 29B: DNA-barcode density investigation. (a) Schematic illustrating how increasing DNA-barcode density enhances oscillation amplitude. Higher barcode density increases negative charge density, stretching the linkers and driving the system toward maximal amplitude under potentials. (b) Initial oscillation amplitudes (dark grayscale) for 0 nM, 20 nM, and 50 nM barcode modifications, and amplitude changes (light grayscale) after incubation with DNA-decode. While the 50 nM condition reaches saturation amplitude, the 20 nM condition maintains optimal dynamic range, providing both sufficient initial amplitude and measurable post-incubation changes for analysis.

FIGS. 30A-30C: Typical scanning electron microscopy images of nanocomplex oscillators at different magnifications. Scale bars: (a) 5 mm, (b) 2 mm, and (c) 500 nm.

FIG. 31: Size distribution of AuNp-virion nanocomplex measured by dynamic light scattering.

FIG. 32: Full uncropped images corresponding to FIG. 27.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Biomolecule: As used herein, “biomolecule” refers to an organic molecule produced by a living organism. Exemplary biomolecules, include without limitation macromolecules, such as nucleic acids, proteins, peptides, oligomers, carbohydrates, and lipids.

Ligand: As used herein, “ligand” refers to a substance that forms a complex with another molecule, such as a biomolecule.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, or any combination thereof.

Protein: As used herein, “protein” or “polypeptide” refers to a polymer of at least two amino acids attached to one another by a peptide bond. Examples of proteins include enzymes, hormones, antibodies, and fragments thereof.

### DETAILED DESCRIPTION

### Introduction

Nanoarray-based assays offer advantages over standard microarray, featuring minimal sample consumption, high efficiency, and enhanced multiplexing capacity. Despite their potential, challenges such as the complexity and high cost of nanoarray spotting and fabrication, along with a lack of effective detection schemes, impede practical applications. Membrane proteins, including G protein-coupled receptors (GPCRs), ion channels, and transporters, representing over 60% of drug targets, pose challenges in extraction, purification, and preserving physiological functions. To address these and other issues, the present disclosure provides a spotting-free nanoarray platform utilizing the virion display technique for study of membrane proteins in some embodiments. Using herpes simplex virus-1 (HSV-1) displayed GPCRs as models, the present disclosure shows that this high-throughput approach enables multiplexed study of membrane proteins under their native conformations on engineered virion envelopes. In some embodiments of this method, DNA-barcoded gold nanoparticle-conjugated virions, anchored on a gold chip via flexible molecular linkers, act as nano-oscillators in an alternating electric field. In some embodiments, the self-assembled nano-oscillators of the present disclosure form a DNA barcoded, spotting-free nanoarray, detected through plasmonic imaging. In some embodiments, this charge-sensitive detection method allows quantitative characterization of small molecule ligand binding kinetics to membrane proteins at the single nano-oscillator resolution. In some embodiments, multiplexed decoding and affinity-addressed DNA barcodes enhance efficiency in this innovative nanoarray platform. This technology opens avenues for drug screening and sensing applications in biological and biomedical research, offering a useful tool for advancing the study of membrane proteins and facilitating drug discovery. These and other attributes will be apparent upon a complete review of the present disclosure, including the accompanying figures.

### Exemplary Methods

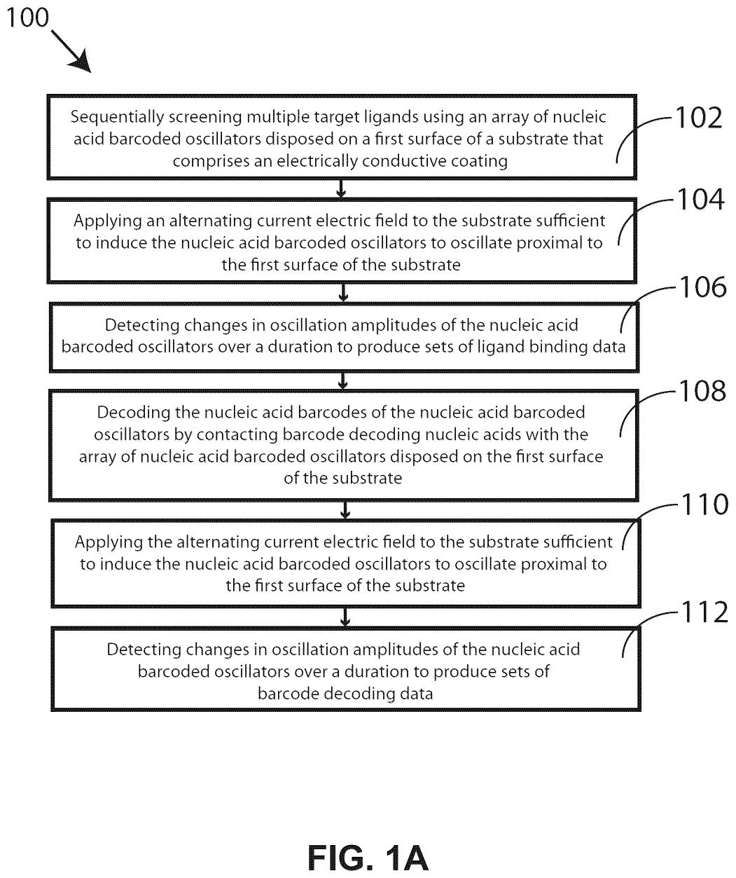

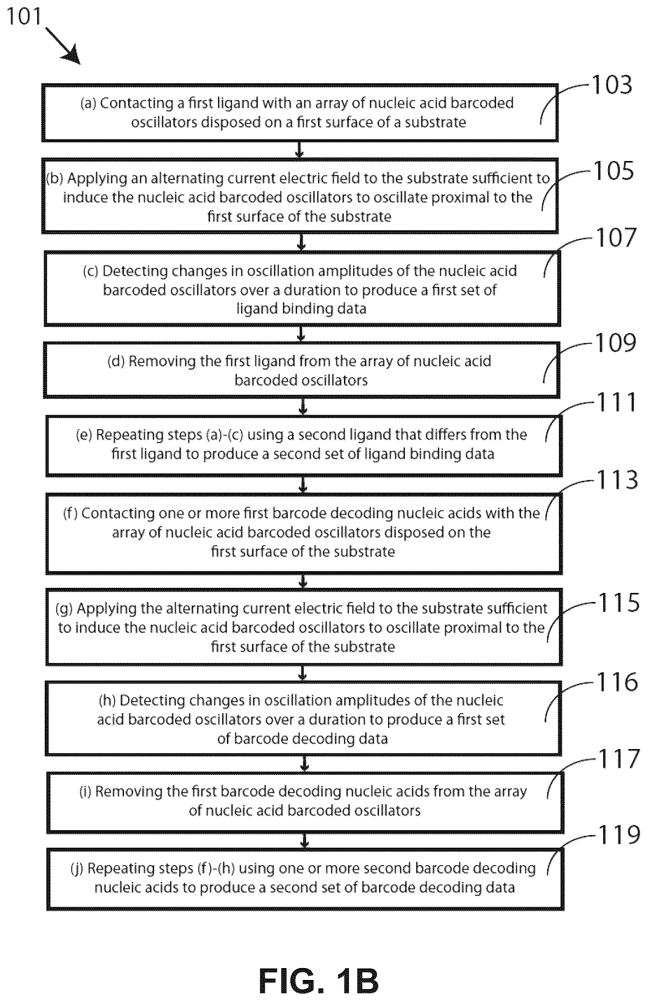

In one aspect, the present disclosure provides a method of performing multiplex detection of ligand (e.g., an antibody, a small molecule, or the like) binding kinetics. As shown in FIG. 1A, for example, method 100 includes sequentially screening multiple target ligands using an array of nucleic acid barcoded oscillators disposed on a first surface of a substrate that comprises an electrically conductive coating (step 102) and applying an alternating current electric field to the substrate sufficient to induce the nucleic acid barcoded oscillators to oscillate proximal to the first surface of the substrate (step 104). Method 100 also includes detecting changes, if any, in oscillation amplitudes of the nucleic acid barcoded oscillators over a duration to produce sets of ligand binding data (step 106). In addition, method 100 also includes decoding the nucleic acid barcodes of the nucleic acid barcoded oscillators by contacting barcode decoding nucleic acids with the array of nucleic acid barcoded oscillators disposed on the first surface of the substrate (step 108), applying the alternating current electric field to the substrate sufficient to induce the nucleic acid barcoded oscillators to oscillate proximal to the first surface of the substrate (step 110), and detecting changes, if any, in oscillation amplitudes of the nucleic acid barcoded oscillators over a duration to produce sets of barcode decoding data (step 112).

To further illustrate, FIG. 1B is a flow chart that schematically shows exemplary method steps of determining binding kinetics of a ligand according to some aspects disclosed herein. As shown, method 101 includes (a) contacting a first ligand with an array of nucleic acid barcoded oscillators disposed on a first surface of a substrate that comprises an electrically conductive coating (step 103). The nucleic acid barcoded oscillators each comprise a nanoparticle attached to the first surface via one or more linker moieties in which at least a first nucleic acid barcoded oscillator comprises one or more first ligand binding moieties and one or more first barcode coding nucleic acids attached to the nanoparticle of the first nucleic acid barcoded oscillator and in which at least a second nucleic acid barcoded oscillator comprises one or more second ligand binding moieties and one or more second barcode coding nucleic acids attached to the nanoparticle of the second nucleic acid barcoded oscillator. The first ligand binding moieties differ from the second ligand binding moieties. The first barcode coding nucleic acids differ from the second barcode coding nucleic acids. In addition, the first ligand is contacted with the array of nucleic acid barcoded oscillators under conditions sufficient for the first ligand to at least partially bind to the first and/or second ligand binding moieties.

Method 101 also includes (b) applying an alternating current electric field to the substrate sufficient to induce the nucleic acid barcoded oscillators to oscillate proximal to the first surface of the substrate (step 105), and (c) detecting changes, if any, in oscillation amplitudes of at least the first and second nucleic acid barcoded oscillators over a duration to produce a first set of ligand binding data (step 107). Method 101 also includes (d) removing the first ligand from the array of nucleic acid barcoded oscillators (step 109), and (e) repeating steps (a)-(c) using a second ligand that differs from the first ligand to produce a second set of ligand binding data (step 111), in which the second ligand is contacted with the array of nucleic acid barcoded oscillators under conditions sufficient for the second ligand to at least partially bind to the first and/or second ligand binding moieties.

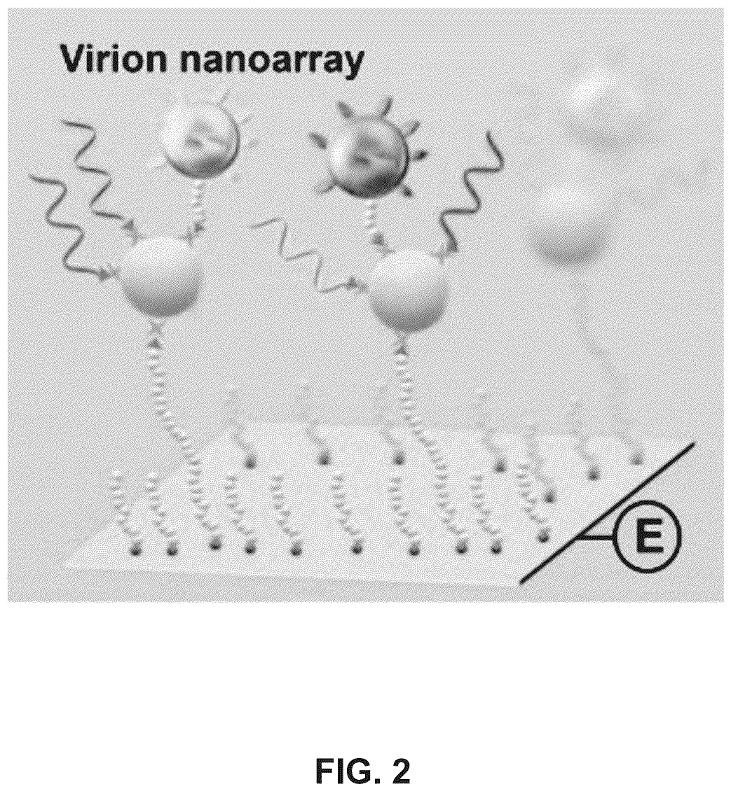

Method 101 also includes (f) contacting one or more first barcode decoding nucleic acids with the array of nucleic acid barcoded oscillators disposed on the first surface of the substrate (step 113), in which the first barcode decoding nucleic acids are at least partially complementary to the first and/or second barcode coding nucleic acids, and in which the first barcode decoding nucleic acids are contacted with the array of nucleic acid barcoded oscillators under conditions sufficient for the first barcode decoding nucleic acids to at least partially hybridize with the first and/or second barcode coding nucleic acids. Method 101 also includes (g) applying the alternating current electric field to the substrate sufficient to induce the nucleic acid barcoded oscillators to oscillate proximal to the first surface of the substrate (step 115), and (h) detecting changes, if any, in oscillation amplitudes of at least the first and second nucleic acid barcoded oscillators over a duration to produce a first set of barcode decoding data (step 116). In addition, method 101 also includes (i) removing the first barcode decoding nucleic acids from the array of nucleic acid barcoded oscillators (step 117), and (j) repeating steps (f)-(h) using one or more second barcode decoding nucleic acids to produce a second set of barcode decoding data (step 119). The second barcode decoding nucleic acids are at least partially complementary to the first and/or second barcode coding nucleic acids. The second barcode decoding nucleic acids differ from the first barcode decoding nucleic acids. In addition, the second barcode decoding nucleic acids are contacted with the array of nucleic acid barcoded oscillators under conditions sufficient for the second barcode decoding nucleic acids to at least partially hybridize with the first and/or second barcode coding nucleic acids. FIG. 2 schematically shows an exemplary virion nanoarray for oscillation measurement according to these methods in some embodiments.

In some embodiments, step (d) comprises washing the first ligand from the array of nucleic acid barcoded oscillators using a buffer. In some embodiments, method 101 includes detecting changes, if any, in oscillation amplitudes of at least the first and second nucleic acid barcoded oscillators over a duration to produce an additional set of ligand binding data prior performing step (e) (e.g., to generate dissociation data). In some embodiments, step (i) comprises washing the first barcode decoding nucleic acids from the array of nucleic acid barcoded oscillators using a buffer. In some embodiments, method 101 includes detecting changes, if any, in oscillation amplitudes of at least the first and second nucleic acid barcoded oscillators over a duration to produce an additional set of barcode decoding data prior performing step (j) (e.g., to generate dissociation data). In some embodiments, the process includes decoding sequence kinetic measurements that start with a buffer to establish a base oscillation amplitude, followed by introducing the barcode decoding nucleic acids to measure association, and then replaced with buffer to measure dissociation. In some embodiments, this process is repeated for next barcode decoding nucleic acids. In some embodiments, an electrical field is applied during the entire measurement process. Starting from introduce buffer (baseline), first ligand (association/binding curve), buffer (dissociation curve). The voltage may not be applied between different ligand samples, while washing off the residue ligand from previous binding with buffer, an optional step.

In some embodiments, the methods disclosed herein include using the first and/or second ligand in a pharmaceutical agent development process based at least in part on the first and/or second set of ligand binding data and the first and/or second set of barcode decoding data. In some embodiments, the methods of the present disclosure include administering the first and/or second ligand to a subject in need thereof based at least in part on the first and/or second set of ligand binding data and the first and/or second set of barcode decoding data.

In some embodiments, the methods disclosed herein include detecting the changes in the oscillation amplitudes of the first and second nucleic acid barcoded oscillators using a plasmonic imaging technique and/or a microscopic imaging technique. In some embodiments, the methods disclosed herein include quantifying the binding kinetics and binding affinity of the first and second ligands using the detected changes in the oscillation amplitudes of the first and second nucleic acid barcoded oscillators over the duration. In some embodiments, the methods disclosed herein include detecting the changes in the oscillation amplitudes of the first and second nucleic acid barcoded oscillators over the duration using a CMOS camera. In some embodiments, method 101 includes removing the second ligand from the array of nucleic acid barcoded oscillators prior to performing step (f). In some embodiments, step (d) of method 101 comprises washing the first ligand from the array of nucleic acid barcoded oscillators.

In some embodiments, the electrically conductive coating comprises gold (Au), indium tin oxide (ITO), silver (Ag), copper (Cu), and/or aluminum (Al). In some embodiments, the linker moieties comprise polyethylene glycol (PEG) moieties and/or biomolecules. In some embodiments, the nanoparticle comprises a metal nanoparticle (MNP). In some embodiments, the nanoparticle comprises a magnetic bead, a polystyrene nanoparticle, or a silica nanoparticle. In some embodiments, the first and/or second ligand binding moieties comprise proteins or nucleic acids.

In some embodiments, the first ligand binding moieties comprise at least a first protein that binds, or is capable of binding, to the first and/or second ligand, wherein the second ligand binding moieties comprise at least a second protein that binds, or is capable of binding, to the first and/or second ligand, and wherein the first and second proteins differ from one another. In some embodiments, virions are attached to the nanoparticles of the first and the second nucleic acid barcoded oscillators and wherein viral envelopes of the virions display the first or second proteins. In some embodiments, the virions comprise human herpes simplex virus-1 (HSV-1) virions. In some embodiments, the first and second proteins comprise different G-protein-coupled receptors (GPCRs).

In some embodiments, the detecting steps (c) and (h) of method 101 comprise introducing an incident light toward a second surface of the substrate to induce a plasmonic wave at least proximal to the first surface of the substrate and detecting a change in intensity of the incident light reflected at an interface of the first surface of the substrate. In some embodiments, the methods include introducing the incident light via at least one objective lens and/or at least one prism. In some embodiments, the methods include introducing the incident light using a superluminescent diode (SLED).

In some embodiments, a first group of barcode coding nucleic acids comprises the first barcode coding nucleic acids and wherein a second group of barcode coding nucleic acids comprises the second barcode coding nucleic acids, in which the first group comprises member nucleic acids having 5, 4, 3, 2, 1, or no non-complementary nucleotides with the first barcode decoding nucleic acid, and in which the second group comprises member nucleic acids having 5, 4, 3, 2, 1, or no non-complementary nucleotides with the second barcode decoding nucleic acid.

In another aspect, the present disclosure provides a method of producing an oscillator array device. The method includes forming an array of nucleic acid barcoded oscillators disposed on a first surface of a substrate that comprises an electrically conductive coating. The nucleic acid barcoded oscillators each comprise a nanoparticle attached to the first surface via one or more linker moieties in which at least a first nucleic acid barcoded oscillator comprises one or more first ligand binding moieties and one or more first barcode coding nucleic acids attached to the nanoparticle of the first nucleic acid barcoded oscillator and in which at least a second nucleic acid barcoded oscillator comprises one or more second ligand binding moieties and one or more second barcode coding nucleic acids attached to the nanoparticle of the second nucleic acid barcoded oscillator. The first ligand binding moieties differ from the second ligand binding moieties. In addition, the first barcode coding nucleic acids differ from the second barcode coding nucleic acids. In some embodiments, the method includes randomly forming the array of nucleic acid barcoded oscillators disposed on the first surface of the substrate.

### Exemplary Devices and Kits

The present disclosure also provides various oscillator array devices and kits. In some embodiments, an oscillator array device of the present disclosure (see, e.g., FIG. 2) includes a substrate that comprises a first surface that comprises an electrically conductive coating and an array of nucleic acid barcoded oscillators disposed on the first surface of the substrate. The nucleic acid barcoded oscillators each comprise a nanoparticle attached to the first surface via one or more linker moieties in which at least a first nucleic acid barcoded oscillator comprises one or more first ligand binding moieties and one or more first barcode coding nucleic acids attached to the nanoparticle of the first nucleic acid barcoded oscillator and in which at least a second nucleic acid barcoded oscillator comprises one or more second ligand binding moieties and one or more second barcode coding nucleic acids attached to the nanoparticle of the second nucleic acid barcoded oscillator. The first ligand binding moieties differ from the second ligand binding moieties. In addition, the first barcode coding nucleic acids differ from the second barcode coding nucleic acids.

In some embodiments, the electrically conductive coating comprises gold (Au), indium tin oxide (ITO), silver (Ag), copper (Cu), and/or aluminum (Al). In some embodiments, the linker moieties comprise polyethylene glycol (PEG) moieties and/or biomolecules. In some embodiments, the oscillator array device further includes one or more spacer moieties attached to the first surface and/or to the linker moieties. In some embodiments, the oscillator array device further includes one or more blocking moieties attached to the first surface.

In some embodiments, the first ligand binding moieties comprise at least a first protein that binds, or is capable of binding, to a first and/or a second ligand, wherein the second ligand binding moieties comprise at least a second protein that binds, or is capable of binding, to the first and/or second ligand, wherein the first and second proteins differ from one another, wherein virions are attached to the nanoparticles of the first and the second nucleic acid barcoded oscillators, and wherein viral envelopes of the virions display the first or second proteins. In some embodiments, the virions comprise human herpes simplex virus-1 (HSV-1) virions. In some embodiments, the first and second proteins comprise different G-protein-coupled receptors (GPCRs). In some embodiments, a kit includes the oscillator array device.

### Exemplary Systems

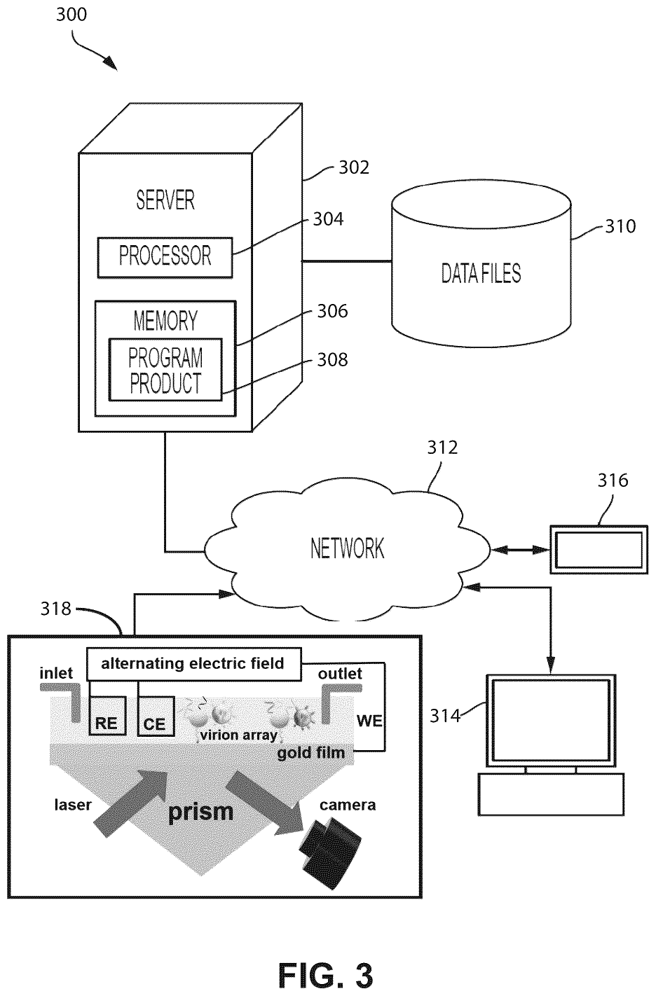

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 3 provides a schematic diagram of an exemplary system suitable for use with implementing at least aspects of the methods disclosed in this application. As shown, system 300 includes at least one controller or computer, e.g., server 302 (e.g., a search engine server), which includes processor 304 and memory, storage device, or memory component 306, and one or more other communication devices 314, 316, (e.g., client-side computer terminals, telephones, tablets, laptops, other mobile devices, etc. (e.g., for receiving molecular interaction data sets or results, etc.) in communication with the remote server 302, through electronic communication network 312, such as the Internet or other internetwork. Communication devices 314, 316 typically include an electronic display (e.g., an internet enabled computer or the like) in communication with, e.g., server 302 computer over network 312 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 300 also includes program product 308 (e.g., for determining binding kinetics of a ligand as described herein) stored on a computer or machine readable medium, such as, for example, one or more of various types of memory, such as memory 306 of server 302, that is readable by the server 302, to facilitate, for example, a guided search application or other executable by one or more other communication devices, such as 314 (schematically shown as a desktop or personal computer). In some aspects, system 300 optionally also includes at least one database server, such as, for example, server 310 associated with an online website having data stored thereon (e.g., entries corresponding to molecular interaction data, etc.) searchable either directly or through search engine server 302. System 300 optionally also includes one or more other servers positioned remotely from server 302, each of which are optionally associated with one or more database servers 310 located remotely or located local to each of the other servers. The other servers can beneficially provide service to geographically remote users and enhance geographically distributed operations.

As understood by those of ordinary skill in the art, memory 306 of the server 302 optionally includes volatile and/or nonvolatile memory including, for example, RAM, ROM, and magnetic or optical disks, among others. It is also understood by those of ordinary skill in the art that although illustrated as a single server, the illustrated configuration of server 302 is given only by way of example and that other types of servers or computers configured according to various other methodologies or architectures can also be used. Server 302 shown schematically in FIG. 3, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 300. As also understood by those of ordinary skill in the art, other user communication devices 314, 316 in these aspects, for example, can be a laptop, desktop, tablet, personal digital assistant (PDA), cell phone, server, or other types of computers. As known and understood by those of ordinary skill in the art, network 312 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 308 is optionally in the form of microcode, programs, cloud computing format, routines, and/or symbolic languages that provide one or more sets of ordered operations that control the functioning of the hardware and direct its operation. Program product 308, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 308 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 308 is optionally copied from the computer-readable medium to a hard disk or a similar intermediate storage medium. When program product 308, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 308 includes non-transitory computer-executable instructions which, when executed by electronic processor 304, perform at least: applying an alternating current electric field to a substrate having a first surface and a second surface opposite the first surface, wherein the first surface comprises an electrically conductive coating and an array of nucleic acid barcoded oscillators attached to the first surface via one or more linker moieties, and wherein the alternating current electric field induces the nucleic acid barcoded oscillators to oscillate proximal to the first surface of the substrate; introducing an incident light toward the second surface of the substrate from a light source to induce the plasmonic wave at least proximal to the first surface of the substrate; and detecting changes in oscillation amplitudes of the first and second nucleic acid barcoded oscillators over a duration.

In some embodiments, binding kinetics of a ligand is measured using device 318. As shown, device 318 includes a substrate (e.g., gold coated coverglass) having first surface and a second surface opposite the first surface. The first surface comprises an electrically conductive coating (e.g., Au) and an array of nucleic acid barcoded oscillators as described herein. Device 318 also includes a power source electrically connected to the substrate. The power source is configured to apply an alternating current electric field to the substrate. Device 318 also includes a prism disposed proximal to the second surface of the substrate. In addition, device 318 also includes a light source configured to introduce light (e.g., collimated light) through the prism to induce a plasmonic wave at least proximal to the first surface of the substrate, and a detector (e.g., a CMOS camera) configured to collect light reflected from the substrate.

### EXAMPLES

### Example 1: Plasmonic DNA-Barcoded Virion Nano-Oscillators for Multiplexed Quantification of Small-Molecule Binding Kinetics to Membrane Proteins

### Introduction

Array-based detection techniques have made remarkable progress in recent years, demonstrating the potential for rapid, high-throughput biochemical screening. Conventional microarray platforms, however, are constrained by spot sizes in the hundreds of micrometers, resulting in limited array density and substantial sample consumption. These inherent limitations have sparked the growing interest in nanoarrays with much smaller dimensions. Current nanoarrays fabrication employs either top-down lithographic nanofabrication techniques (e.g., dip-pen nanolithography, electron beam lithography, and nanoimprint lithography) or bottom-up nanopattern techniques (e.g., DNA origami). While these methods can achieve highly ordered arrays with feature sizes down to 100 nm, they remain limited by their dependence on expensive lithography equipment and/or labor-intensive fabrication steps. Moreover, existing readout approaches for ultra-miniaturized arrays face significant limitations. Fluorescence-based detection, for instance, relies on fluorophore labeling, which may perturb native molecular interactions and is further compromised by photobleaching-induced signal decay. Alternatively, scanning probe microscopy techniques (e.g., Kelvin probe force microscopy and atomic force microscopy) are hampered by slow acquisition rates due to their sequential scanning nature, while the required tip-sample interactions risk mechanical or chemical degradation of delicate biomolecular structures.

In contrast, self-assembled nanoparticles offer the potential for high-density, parallel measurements similar to nanoarrays, while simplifying both fabrication and measurement. The plasmonic effect allows individual nanoparticles to serve as sensing units, as the localized surface plasmon resonance (LSPR) wavelength shifts in response to bound proteins. However, a key challenge arises in multiplexed detection: unlike pre-patterned arrays with defined spatial arrangements, randomly distributed nanoparticles lack inherent positional encoding for target identification. One approach to nanoparticle identification involves continuously recording immobilization positions, where different types of biofunctionalized nanoparticles are adsorbed onto the chip sequentially, one type at a time. However, this slow, batch-by-batch adsorption process for chip fabrication and detection reduces efficiency, hindering high-throughput screening of large biochemical libraries and restricting its practical use.

For individual nanoparticle sensing, we have developed the nano-oscillator technique, based on surface plasmon resonance (SPR) microscopy, to image nanoparticles tethered on a gold substrate. In this method, charged nanoparticles are driven into oscillation by an external alternating electric field, and the oscillation amplitude, quantified by the plasmonic image intensity changes, is proportional to the net charge of the nanoparticle. Unlike conventional mass-sensitive techniques, such as SPR and LSPR, in which the signals are scaled and constrained by the molecular weight of analytes, the nano-oscillator focuses on detecting charge changes. This feature makes the technique versatile for studying biomolecular interactions, as charge distribution is altered in most biomolecular interactions, including those involving small molecules. Using this strategy, we have successfully measured peptide phosphorylation dynamics and small-molecule binding kinetics. Nevertheless, multiplexed detection has remained elusive.

Small molecules and peptides are the most popular drug candidates. In 2021, over two-thirds of the new approved drugs were small molecules and peptides. Measuring their binding kinetics to their target is an essential step in drug discovery. However, due to their low molecular weight, small molecules pose challenges for detection using common mass-based label-free methods, especially for their interactions with large protein receptors. Conversely, labeling the small molecules with fluorescence or chemiluminescence dyes could improve sensitivity but may alter their sizes and structures, potentially affecting their properties and functions. Thus, label-free detection methods are critical for accurate characterization of small-molecule interactions. Our nano-oscillator sensor addresses this challenge by detecting ligand-binding events through charge modulation rather than mass changes, enabling direct observation of unmodified small molecules. This approach preserves intrinsic binding kinetics while maintaining high sensitivity. We envision that the nano-oscillator technique, leveraging single nanoparticle resolution and widefield imaging, could enable high-throughput detection of small molecule binding kinetics in a nanoarray-like format. However, realizing this vision requires breakthroughs in robust nano-oscillator fabrication, reliable measurement of biomolecular interactions at the individual nano-oscillator level, and effective addressing of each nano-oscillator on the sensor chip.

In this example, we report a new high-density nanosensor platform created by self-assembled, DNA-barcoded nano-oscillators that enables multiplexed molecular detection without nanolithography or precision patterning. This innovative multiplexing capability represents a significant technological advancement, allowing for high-throughput analysis of molecular interactions while maintaining single-nanosensor resolution in a highly scalable platform. To showcase this breakthrough, we developed a nano-oscillator sensor for parallel profiling of G protein-coupled receptors (GPCRs), the largest human membrane protein family (>800 members) and crucial drug targets that are notoriously difficult to study due to their structural fragility. We use virion display technology to preserve native GPCR conformation, and combine it with the plasmonic detection platform and DNA-barcodes to achieve label-free, multiplexed measurements of small-molecule binding kinetics with GPCRs. A unified charge-sensitive transduction mechanism of nano-oscillators is used to detect ligand-binding events and resolve affinity-specific DNA barcode hybridization for target discrimination. Five small-molecule ligands, including neurotensin (a 13-residue peptide modulating neurotransmission and cancer progression via NTSR1 receptor interactions) and other clinically relevant compounds (e.g., kinase inhibitors or GPCR-targeting drugs), and their corresponding GPCR receptors, are used as models in this study. Our results show that this plasmonic DNA-barcoded virion nano-oscillator sensor platform has great potential for high-throughput screening of molecular interactions in biological, biochemical, and biomedical applications, addressing a critical need in modern drug discovery and biomaterial development.

### Results and Discussion

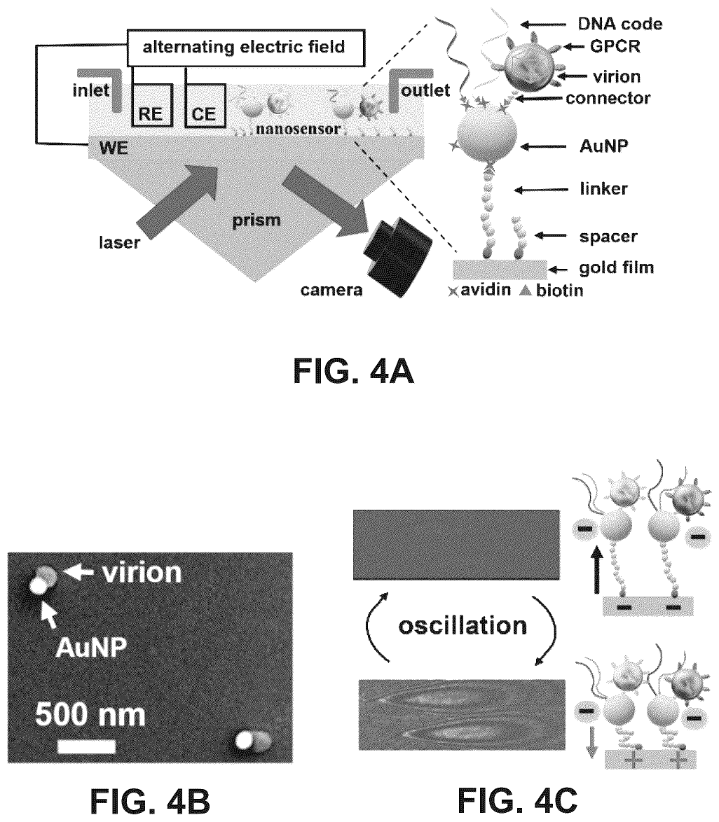

To overcome the limited imaging throughput of objective-based SPR microscopy used in our previous studies, we use a prism-based Kretschmann configuration with an expanded field-of-view for high-throughput plasmonic imaging (FIG. 4a; see Experimental Setup in Supplementary Information). In this platform, each surface-assembled nano-oscillator functions as an independent sensing unit, facilitating multiplexed detection in a nanoarray fashion. By integrating a multi-state encoding-decoding strategy that leverages DNA sequence-specific hybridization affinities, individual nano-oscillators can be uniquely identified, permitting massively parallel measurements.

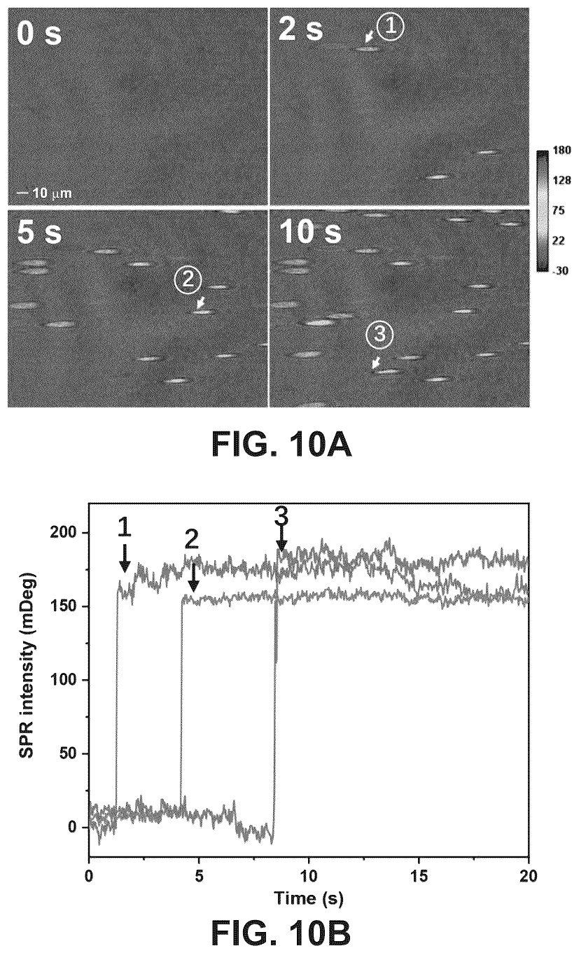

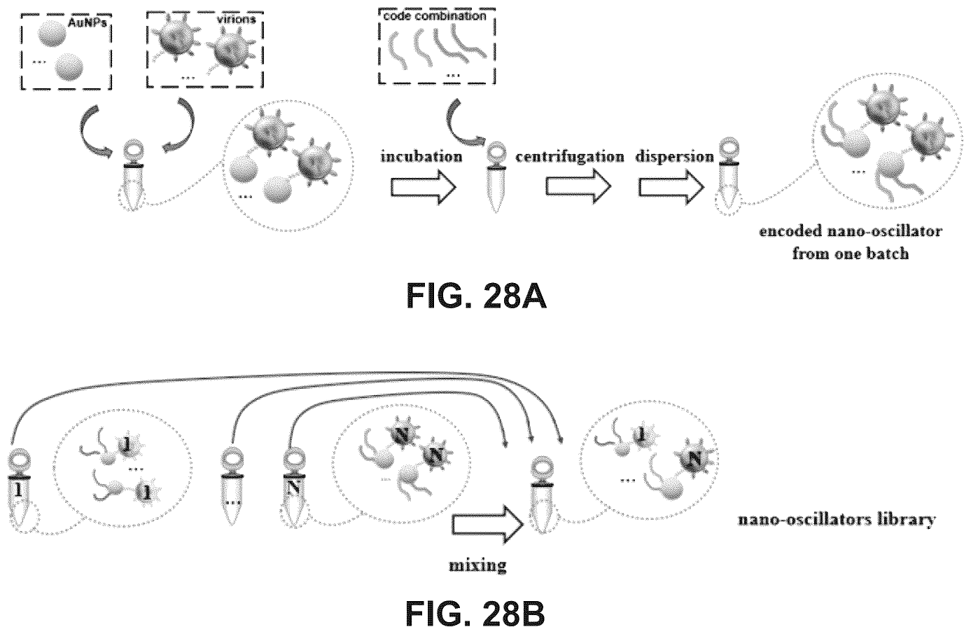

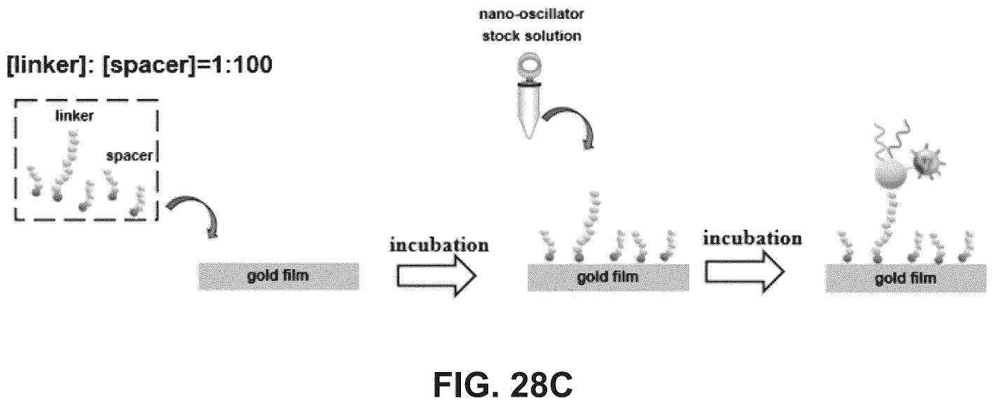

We present a nano-oscillator featuring a nanocomposite structure (FIG. 4a inset), where a 150 nm gold nanoparticle (AuNP) is linked to a GPCR-displayed virion (herpes simplex virus-1, ˜200 nm), amplifying the oscillation response and providing additional docking sites for multiplexed identification encoding (see scanning electron microscopy image in FIG. 4b and detailed descriptions in FIGS. 10-13 and 27-31). A library of nanocomposites was created, with each type of nanocomposite consisting of a gold nanoparticle conjugated to a virion expressing a unique GPCR (FIGS. 28a and 28b). GPCR-displayed virions were conjugated to streptavidin-coated AuNPs using a bifunctional polyethylene glycol (PEG) molecule. The PEG molecule has N-hydroxysuccinimide (NHS) at one end and biotin at the other, enabling it to bind to primary amines on the virion envelope and streptavidin on the AuNPs. Concurrently, biotinylated DNA barcode sequences were immobilized on the streptavidin-coated AuNPs. In this way, the DNA on the AuNP marks the specific type of virion to which it is conjugated. Nanocomposites were self-assembled onto a gold film modified with long-chain thiol-PEG-biotin linkers, where the biotin group binds to streptavidin on the nanocomposite. A substantial quantity of short spacer-PEG was employed to regulate the linker density and minimize non-specific adsorption. The long (˜60 nm) and flexible PEG linker effectively tethers the nanocomposites without obstructing their oscillation under the alternating electric field, thereby enabling the creation of efficient nano-oscillators (FIG. 28c).

Instead of imaging via a high numerical aperture oil-immersion objective based SPR microscopy, a prism-based Kretschmann configuration setup was used for large field-of-view plasmonic imaging. Each nano-oscillator locates within the plasmonic image is served as an independent sensing unit, enable simultaneous multiplexed detection in a nanoarray fashion. Nano-objects surrounded by the plasmonic wave induce a scattering component that interferes with the plasmonic wave and generate a parabolic shape pattern in the SPR image. This interference enhanced signal makes nanoparticles smaller than the diffraction limit visible in SPR imaging. Owing to the exponentially decaying nature of plasmonic wave intensity with increasing distance from the surface, the imaging signal has remarkable sensitivity to the vertical displacement of nano-oscillators relative to the sensor interface. When driven by an external alternating electric field, perpendicular oscillation of nano-oscillators with respect to the interface induces periodic intensity modulations in the plasmonic imaging signal. As a result, the oscillation amplitude of the nano-oscillator (x₀) could be accurately measured by monitoring the oscillation-induced intensity change in SPR image. Subsequently, the net charge of nano-oscillator (q) can be derived by the oscillation model, given by equation (1):

$${x}_{0} = \frac{{E}_{0}}{j 12 {π}^{2} η af} q \qquad ( 1 )$$

where E₀ and f are the amplitude and frequency of the applied electric field, η is the viscosity of the solution, a is the radius of nano-oscillator, and j is the imaginary unit (j=\sqrt{−1}) to represent a 90° phase difference between the applied electric field and oscillation displacement. Consequently, the charges of the nano-oscillator can be conveniently monitored by its intensity in SPR images, and changes of charges induced by molecule interactions can be recorded in real-time to quantify binding kinetics.

In this example, GPCR-displayed virions were assembled into nano-oscillators to characterize binding kinetics of small molecule drugs with human GPCRs. A 150 nm gold nanoparticle (AuNP) is conjugated to a GPCR-displayed virion (˜200 nm), forming a nanocomposite structure, to amplify the oscillation response and to provide extra dock for multiplexing identification label (see scanning electron microscopy image in FIG. 4C and detailed descriptions in FIGS. 10-13). A library of nanocomposite containing different GPCRs were generated as the following: the GPCR-displayed virion and the streptavidin coated AuNP was conjugated by a functional polyethylene glycol (PEG) molecule with N-hydroxysuccinimide (NHS) and biotin ends, which can bind to primary amines on the envelope of the virion and streptavidin on the AuNP, respectively. At the same time, biotin-modified DNA barcoding sequences also were immobilized to the streptavidin on the AuNP to mark the specific type of GPCR-displayed virion, for subsequence in-situ decoding using the plasmonic imaging-based detection of DNA hybridization.

The random array of nanocomplex-oscillators from the library were formed on sensor surface by self-assembly. The assembly was coupled by a long chain thiol-PEG-biotin molecule with a molecular weight of 10 kDa, the thiol and biotin will bind to the gold sensor surface and the streptavidin coated AuNP respectively and then tether the nanocomplex near the sensor surface. To minimize nonspecific interaction onto the exposed gold surface, the substrate was also blocked by short-chain thiol-(PEG)4-methoxy molecule. The linker PEG molecule was designed to be long (˜60 nm in length) and flexible enough to tether the nanocomplex on the substrate but avoid impeding its oscillation under the alternating electric field. Since the nanocomplexes were negatively charged, they were attracted to the substrate under the positive potential with the enhanced signal and were repelled to the solution under the negative potential with the decreased signal, which were precisely measured by the distance-sensitive plasmonic images (FIG. 4C).

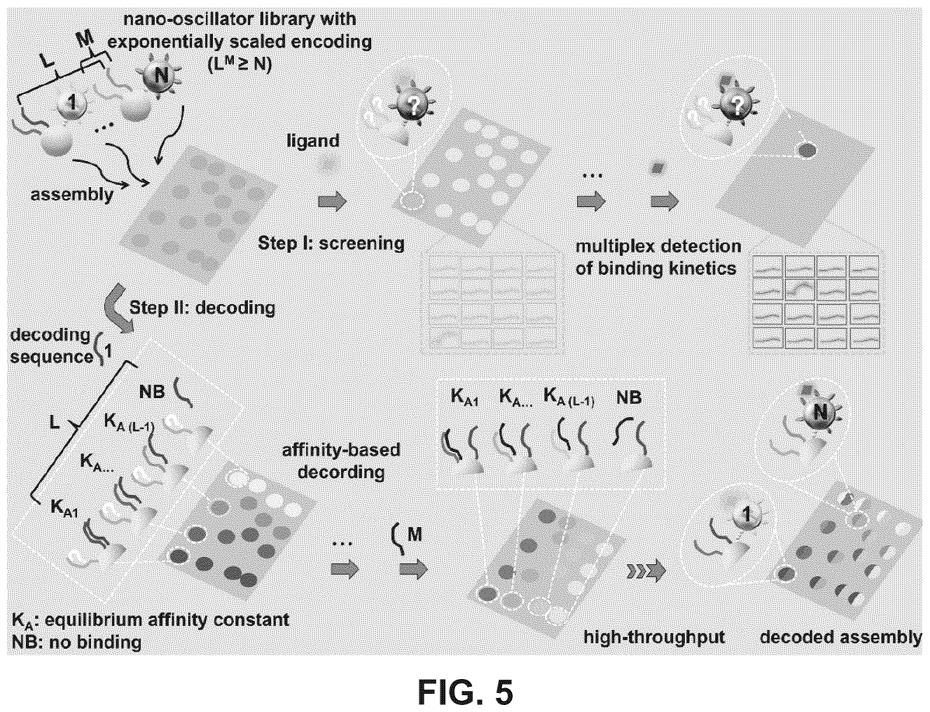

The workflow of the nanoarray measurement is shown in FIG. 5. After the nanocomplex-oscillator array from the library is assembled on the sensor surface, it is ready for the multiplexing detection. The first step of the measurement is target screening. By sequentially delivering small molecule drugs (ligands) onto the nanoarray and following with buffer washing, ligands-GPCRs binding and dissociation induce changes of charges on each individual oscillator are reflected by its oscillation amplitude change, which are quantified with changes of plasmonic image intensity. The binding kinetics between ligands and GPCRs can then be determined from the response curves extracted from the images. After the binding kinetic measurements are finished, the decoding processes is performed by hybridizing the specific readout sequence to the barcodes on the nanocomplex. Same as the measurement of ligand binding kinetics, the decoding hybridizations induced changes of charges on individual oscillators are quantified by the changes of their oscillation amplitudes.

Following ligand binding kinetics measurements, the nano-oscillator system detects DNA hybridization events through the intrinsic charge signal of oligonucleotides and performs decoding via an affinity-based encoding-decoding scheme. In this process, specific readout sequences undergo sequential hybridization with the DNA barcodes on each nano-oscillator. The system distinguishes each barcode, whether fully complementary or containing nucleotide mismatches to the readout sequence, through its characteristic hybridization affinity, enabling precise classification into distinct groups. Consider a system with M barcoding groups (each addressed by a unique decoding sequence), where each group contains L possible DNA barcode states (including the absence of a barcode as a state). This configuration generates L^{M} distinct combinatorial encoding states.

Conventional DNA barcoding (where L=2, detecting only presence/absence of a barcode) offers limited encoding capacity. In contrast, our affinity-assisted DNA barcoding approach significantly expands this capacity by correlating binding kinetics with hybridization mismatches, allowing the inclusion of additional mismatched barcodes and thus increasing L. Through M sequential decoding hybridizations, this method provides (L−2)^{M} fold greater encoding states compared to conventional binary barcoding. This exponentially scaled capacity enables the encoding of a much larger library of nano-oscillators. The decoding process is also streamlined, requiring only a limited number of hybridization steps to achieve comprehensive barcode identification.

Crucially, the platform's multiplexing capability is enhanced by its dual-use detection scheme: a single charge-sensitive transduction system resolves both DNA barcode signatures and ligand binding interactions. With integrated encoding, decoding, and ligand screening in a unified nano-oscillator platform, this innovative strategy achieves unprecedented efficiency and establishes a new paradigm for multiplexed analysis.

The DNA barcodes are decoded based on the hybridization affinity to the readout sequences. The barcodes are divided into groups by a distinct decoding sequence. In each group, a barcode has a sequence that is complementary or has a couple of mismatched nucleotides to the decoding sequence. While barcodes in different groups are identified by different readout sequences, barcodes within a group are discriminated by the affinities to the readout sequence since the binding kinetics are corelated to the number of mismatches in hybridizations. In this way, each group is decoded just by one time hybridization of a single decoding sequence, significantly reduced the decoding time and cost.

Assuming each coding group contained j types of DNA barcodes (i.e. states, and noting no barcode could also be considered as an individual state), and the number of coding groups was m, the combinatorial assignment of DNA barcodes from different coding groups would generate distinct states of j^{m}. For conventional DNA barcoding strategy, where only with and without barcode could be distinguished by one readout, j=2. If we additionally introduce 2 different mismatched barcodes, then j becomes 4 (with/without and 2 mismatches). Therefore, for m groups of decoding sequences, the affinity assisted DNA barcoding can potentially have 2^{m} times more distinct states than conventional method. This exponentially scaled capacity can effectively encode a large library of nano-oscillators. The decoding process is streamlined and efficient, requiring only a few sequential hybridizations. And the readout is facilitated by the same charge sensitive transduction mechanism for the ligand bindings. This innovative approach significantly enhances the encoding and decoding capabilities of DNA barcoding, providing a powerful tool for efficiently handling diverse libraries of nano-oscillators.

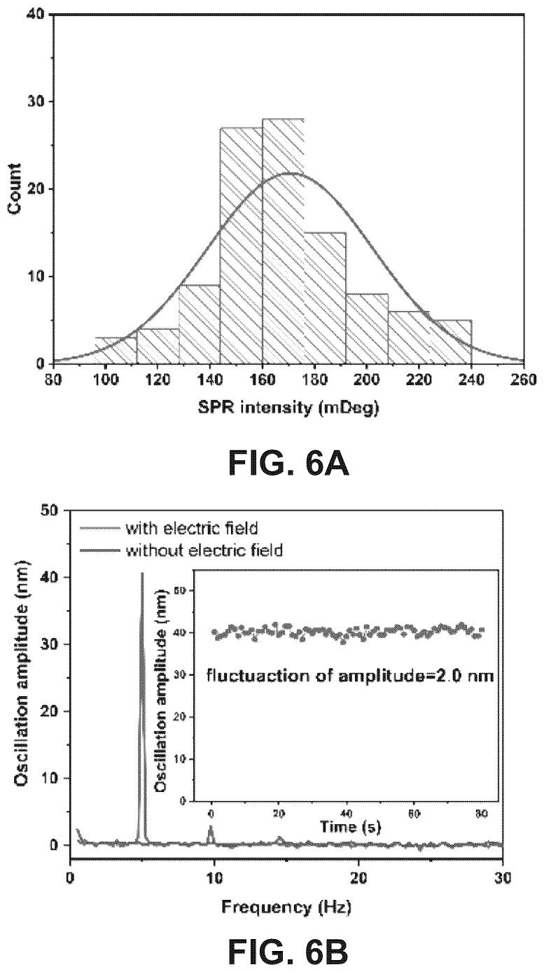

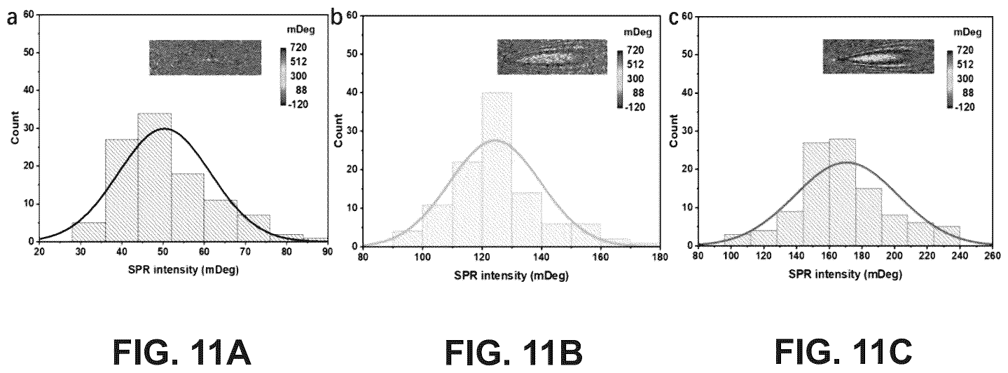

Virion-AuNP nanocomplex not only acted as the carrier for membrane proteins and DNA barcodes, but also played the role to generate the signal in plasmonic imaging. An 3× contrast enhancement can be observed due to the higher refractive index of the nanocomplexes (FIG. 11). As shown in FIG. 6A, the histograms of 106 individual nanocomplex oscillation events can be fit with a single Gaussian distribution peak, which indicates that the self-assembled nanoarray was reliably fabricated with no observable aggregations. The oscillation amplitudes of individual oscillators were extracted from the Fast Fourier Transform (FFT) plasmonic imaging sequence. As shown in FIG. 6B, when the averaged FFT image intensity of a nano-oscillator was plotted as a function of frequency, a pronounced peak at the frequency of the applied electric field (5 Hz) is observed. An oscillation amplitude of 40 nm was achieved for the nanocomplex (lighter grayscale curve) with the applied electrical field. The uncertainty of the detection was determined by testing the signal component at the frequency of 5 Hz without applying electric field (darker grayscale curve), which could be attributed to the Brownian motion induced distance changes and the measurement errors. For the measurement of the nanocomplex, the uncertainty was 0.36 nm. The time-dependent oscillation behaviors were recorded by measuring the oscillation amplitudes over one minute (Insets of FIG. 6B). For the nanocomplex, the mean value of amplitude was 40.2 nm and a fluctuation of 2.0 nm were recorded, which corresponded to charges of 380 e and 18.9 e, respectively, according to equation (1). For a surface with N electron charges, the charge fluctuation is in the order of \sqrt{N},^{[14]} and thus the estimated charge fluctuation for the nanocomplex is \sqrt{380} e=19.4 e, close to the measured value of 18.9 e. This result show that the intrinsic charge fluctuation can be effectively monitored by the nano oscillators, and it is the major origin of amplitude fluctuations that should be taken into account in amplitude measurements. In the following study, to ensure the reliable measurement of targets-induced responses, for nanocomplexes with 40 nm initial oscillation amplitude, only oscillation amplitude changes larger than 3.1 nm were considered as effective responses, which was determined by the sum of charge fluctuation induced amplitude changes (2.0 nm) and 3-fold of the uncertainty in measurement (3×0.36 nm).

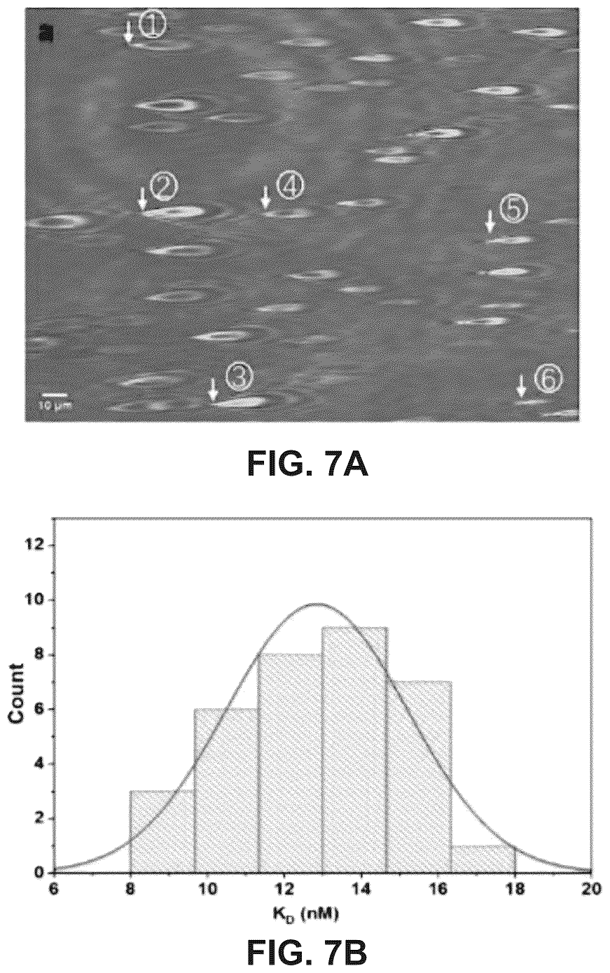

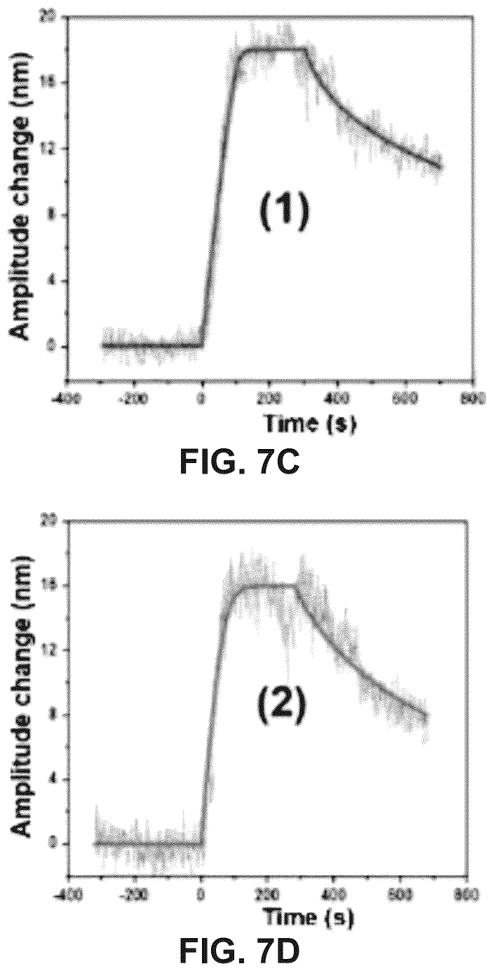

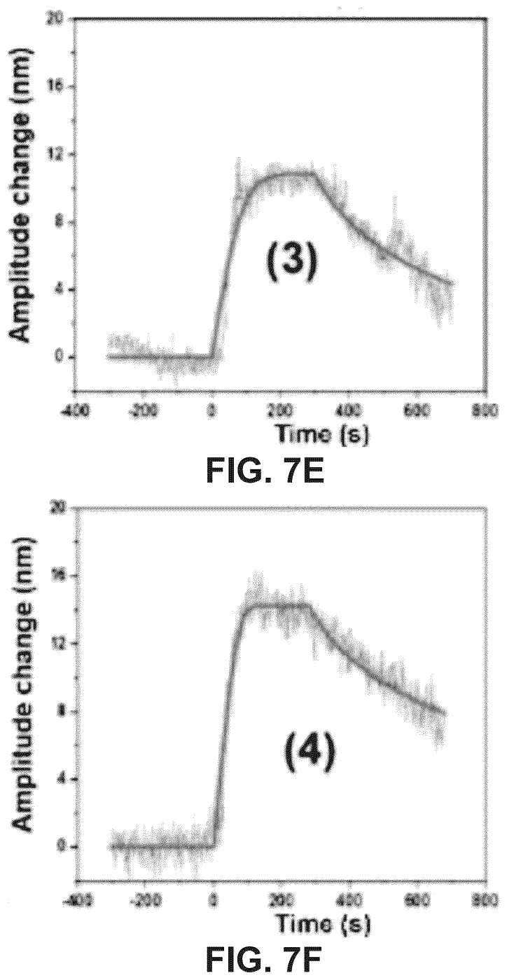

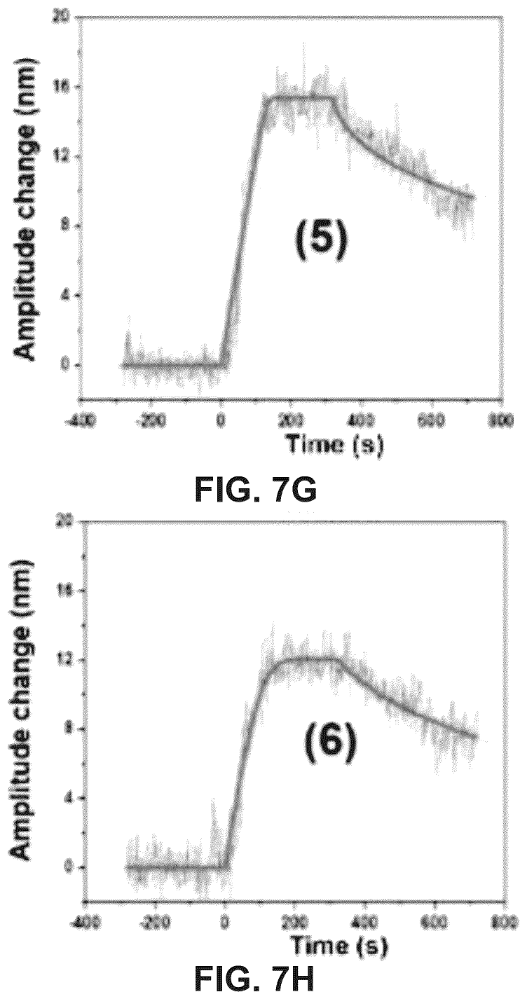

While macromolecular drugs are on the raise, small molecules and peptides are still the most popular drug candidates. Over two-third of new approved drugs by FDA in 2021 are small molecules and peptides. Due to their low molecular weight, small molecules pose challenges for detection using mass-sensitive methods, especially in interactions with large protein receptors. Additionally, labeling them with fluorescence or chemiluminescence dyes may alter the molecule's size, potentially affecting its properties and functions. Our nano-oscillator array solves this small molecule detection dilemma by measuring small molecule binding induced charge changes instead of mass changes. To evaluate the capability of the nano-oscillators for quantification of small molecule interaction with macromolecule receptors, nanocomplexes with single virions were firstly studied. Representative results of small molecule ligand binding to a virion displayed GPCR receptor, NTSR1, are shown in FIG. 7. Nanocomplexes with NTSR1-displayed virion were immobilized on sensor surface and driven into oscillation, and the small molecule ligand, neurotensin, was injected and the oscillation amplitude was monitored in real time. The nano-oscillator amplitude changes were extracted from the recorded plasmonic images for tracking association and dissociation responses of individual nano-oscillators. Attributed to the array format, parallel detection was achieved with multiple nanocomplexes imaged within the field-of-view simultaneously.

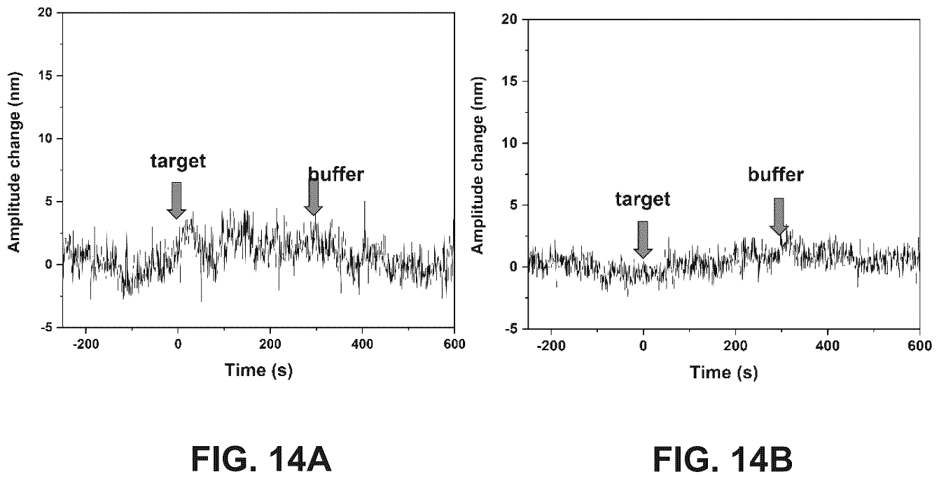

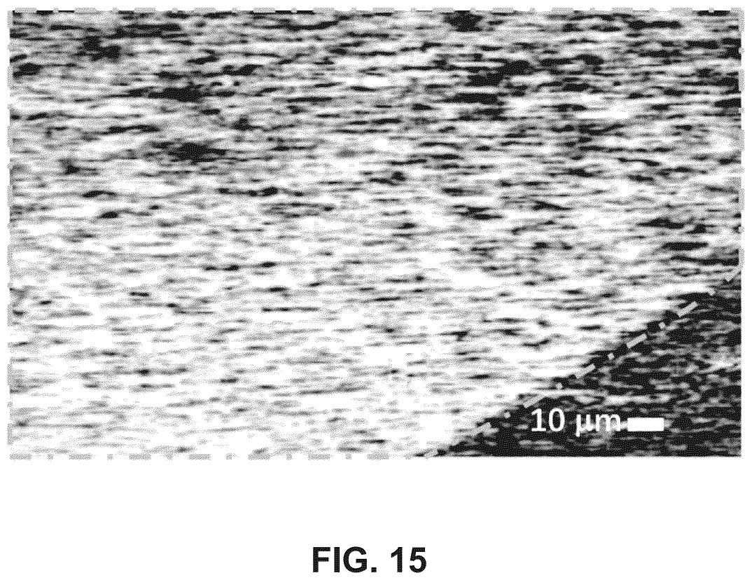

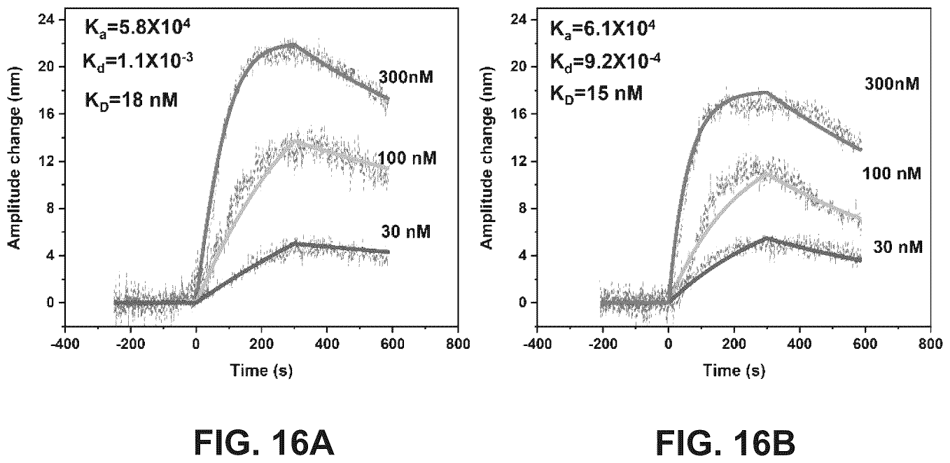

Binding response curves of nano-oscillators numbered in FIG. 7A are plotted in FIGS. 7C-H. Obvious amplitude changes in the presence of the ligand target indicated the interaction between the ligand and the nano-oscillator, which can be used to determine the binding kinetics. By fitting the binding curves with the first order kinetics model, the associate rate constant, dissociate rate constant and equilibrium constants for each nano-oscillator were obtained. From parallel counting of 35 individual nano-oscillators on the sensor chip, an equilibrium dissociation constant (KD) value of 12.8±2.3 nM was derived through Gaussian distribution fitting of the KD values. (FIG. 7B). Control experiments showed no responses for AuNPs without virions or the virions not displaying GPCRs (FIG. 14), which further confirmed that the observed amplitude changes are attributed to the specific interactions between GPCRs and the corresponding ligands. To validate the individual nano-oscillator results, interactions of the same ligand with high density ensemble virion-oscillators were measured (FIG. 15). The binding response curves are the averaged oscillation amplitude of the entire ensemble area as a function of time. The binding kinetics and affinity parameters obtained through the binding curves of the ensemble virion oscillators were consistent with the ones obtained through the binding curves of individual nano-oscillators in the dose-dependent measurements (FIG. 16), demonstrating that the bio-functionality of GPCRs were maintained well in the nanocomplex design. On the other hand, as shown in FIGS. 7C-7H, among individual nano-oscillators, variation of oscillation amplitudes changes in responding to the same ligand were present. This phenomenon is likely due to the heterogeneity in GPCR expression level among individual virions, which will affect maximum binding signal, but not the binding kinetics. This result shows that the nano-oscillator array can reliably measure ligand-GPCR interactions at single oscillator resolution.

To enable affinity-based barcoding, it is important to ensure nanoarrays are addressable by distinct affinities induced by different nucleotides mismatches. To prove the feasibility of the decoding strategy for nanoarray, two decoding sequences are tested. Perfectly matched, two and three-nucleotide mismatched complementary single DNA strains were used as barcode sequences on the nanocomplexes. Attributed to the charge-sensitive detection capability of nano-oscillators, hybridization of the charge-rich decoding DNA onto the nano-oscillators were recorded in real-time, enabling the calculation of hybridization affinities. These affinities serve as discriminative indicators, allowing for the differentiation of nano-oscillators encoded by barcode sequences that vary by just a few nucleotides.

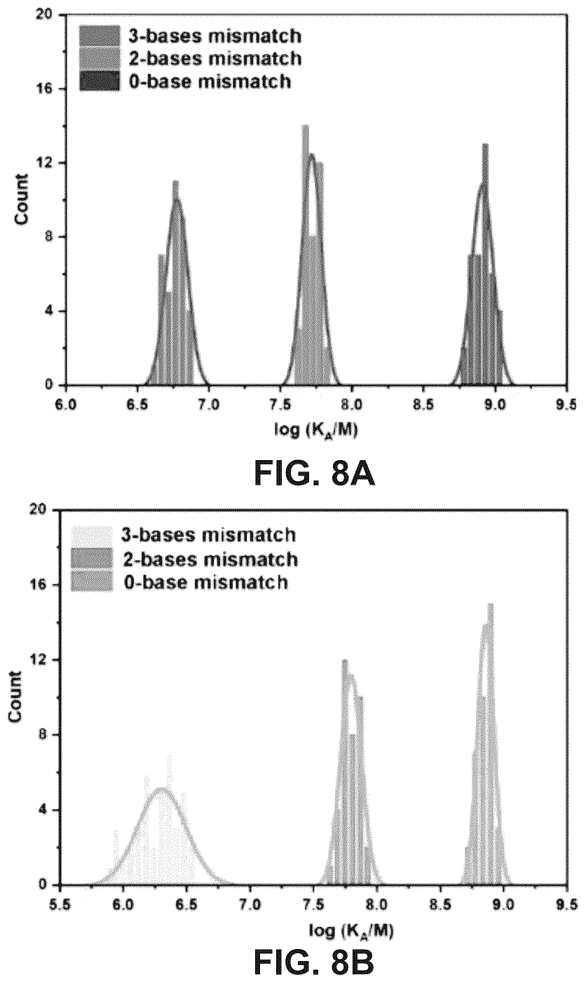

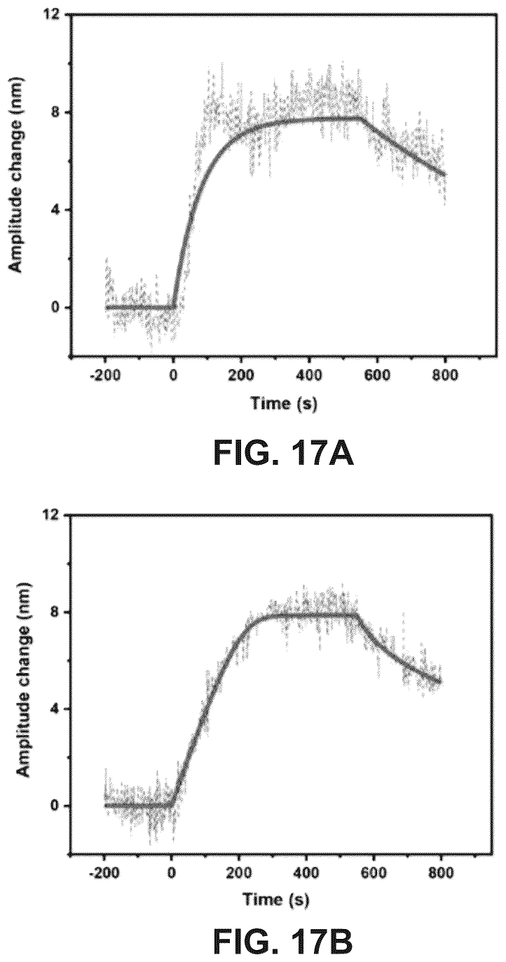

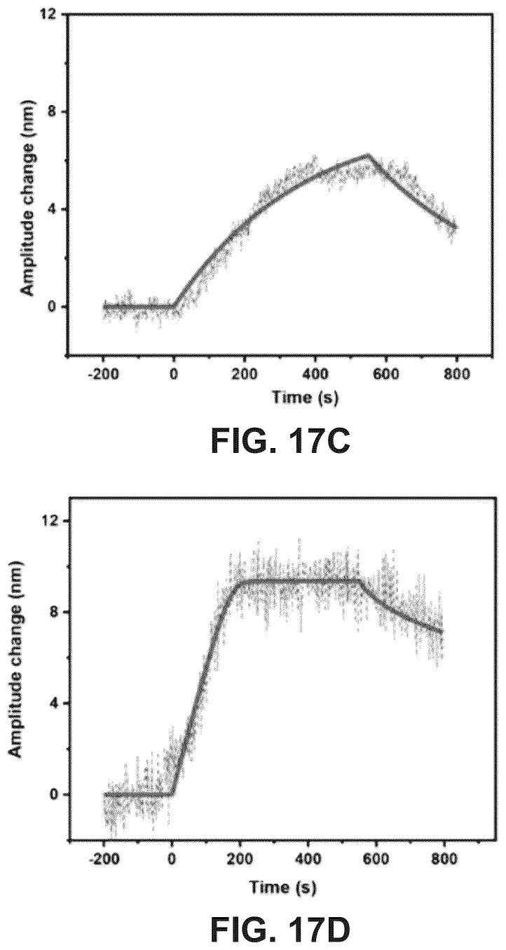

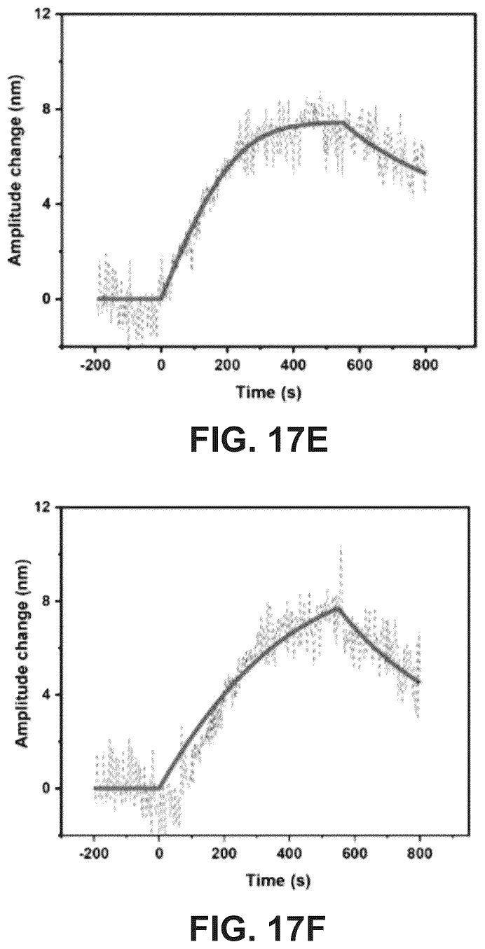

Barcode readout tests in single nano-oscillator with one kind of barcode sequence modification are shown in FIG. 17. The obvious amplitude changes from the decoding process indicated the capability to monitor hybridization in a single nano-oscillator level. In FIG. 8, for each kind of barcode sequence, 37 individual nano-oscillators were measured for statistical study. Distinct affinity differences (>10 fold) were obtained among the hybridizations of decoding-coding pairs containing 0-, 2-, and 3-nucleotide mismatches with narrow distributions, clearly indicating the discriminated encoded states. This result demonstrates that the affinity-based decoding enables simultaneous readout of multiple nucleotides mismatch encoded states, and does not need a separate readout method or complicated nano-barcodes synthesis and labelling. This affinity enhanced DNA barcode strategy only need a few decoding sequences to efficiently readout a large nano-oscillators array for screening applications.

Considering the inevitable presence of degraded virions or imperfectly functionalized nanocomplexes, redundant measurements on multiple particles for the same binding pair are necessary to ensure the quality of the measurement. Supported by this large field of view imaging system, simultaneous measurement of large number of nano-oscillators can be conveniently performed. The responses of nano-oscillators to their specific targets were separately recorded, and the minimal number of particles Nₘᵢₙ needed to ensure an effective response in every measurement was evaluated by the following formula:

$${N}_{min} \geq {( \frac{2 S . D .}{Med - Lim} )}^{2} \qquad ( 2 )$$

in which Med is the median of the measured responses, Lim is the limit of effective responses, and S.D. is the standard error by counting the measured responses. When the median exceeded the limit of effective response by a two-fold of standard error, it had a confidence level of 95% to ensure a significant response just by detecting particles in a number of Nₘᵢₙ. In these tests, based on the statistics in Table 3, Nₘᵢₙ was determined to be between 1 to 3 for the investigated detection. Given that each particle featuring the plasmonic parabolic pattern occupies 250 pixels and the current imaging area comprises 316,224 pixels, this suggests a reasonable sample capacity to simultaneously sense over 1200 particles in the array. Assuming Nₘᵢₙ for each virion displaying a defined GPCR is set to 3, this translates to the potential detection of 400 different kinds of GPCRs. This capacity opens the possibility for screening a large library of in-vitro displayed membrane proteins, such as the recently developed viron displayed library containing 315 kinds of human non-odorant GPCRs.

To demonstrate the feasibility of multiplexing measurements of various ligand-membrane protein interactions, the nano-oscillators conjugated with the defined virions were self-assembled on the gold chip to form a nanoarray. The array contained six types of virions, including five types of GPCRs-displayed virions and one blank control virion without any transfected GPCRs. The combinations of the two pre-tested DNA barcode groups (see FIG. 8) were used to encode the nanocomplex element in the array. Through two rounds of hybridization with four distinct states (three affinity states mediated by barcodes and one no barcode state) per hybridization, the number of the coding combinations is able to cover the six types of virions in the array (4²=16, with ten coding combinations left out). The GPCR-targeted ligands, including peptides and small molecules, were separately flowed over the nanoarray sensor chip, and the real time recorded plasmonic images of nano-oscillators were used to screen the ligands interacted with GPCRs and determine the binding kinetics. Followed by introducing two decoding DNA sequences sequentially, individual nano-oscillators were identified by the combination of the affinity characterization.

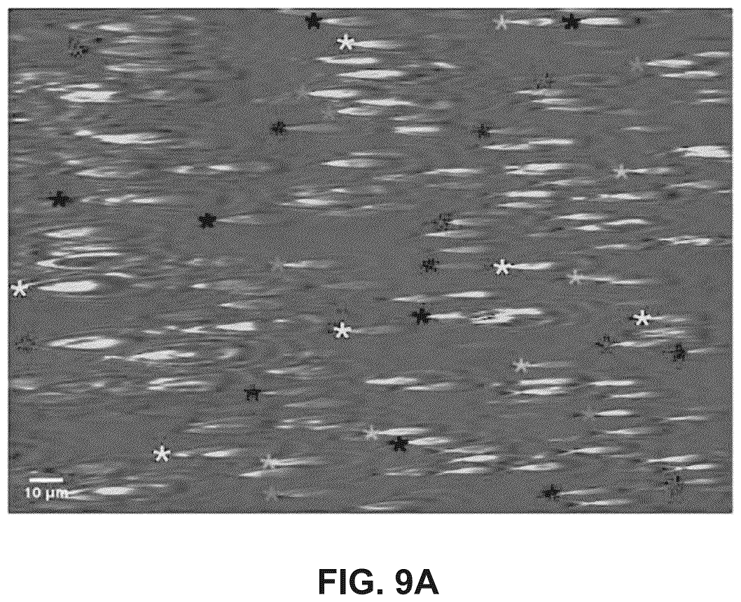

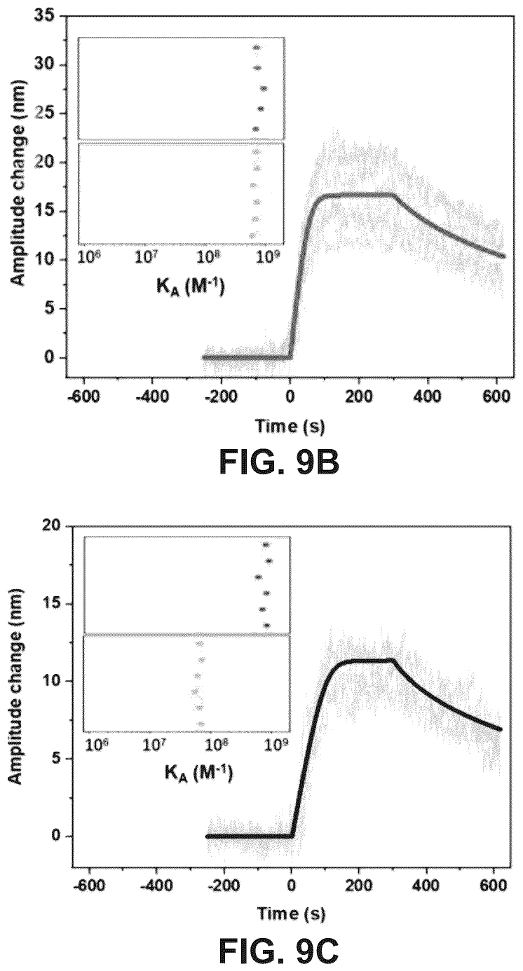

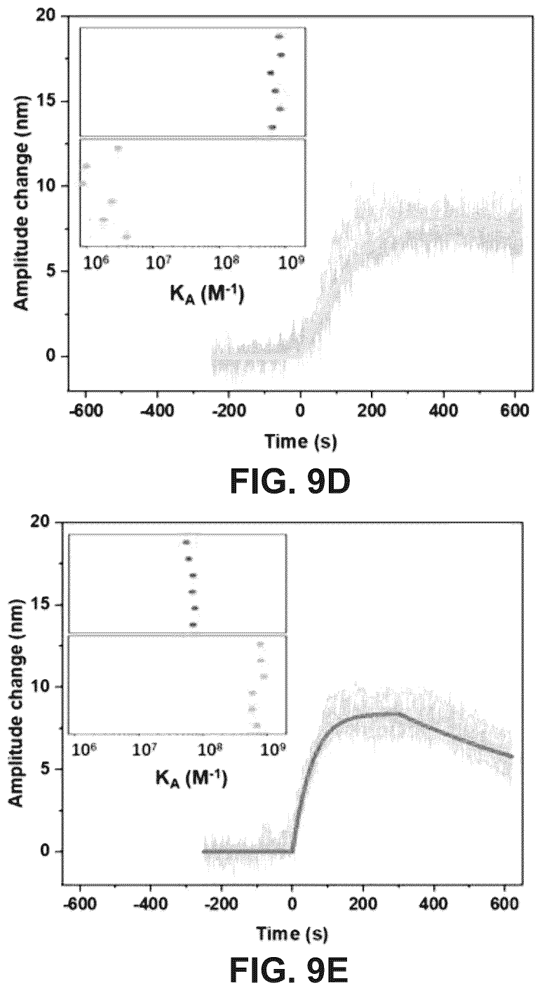

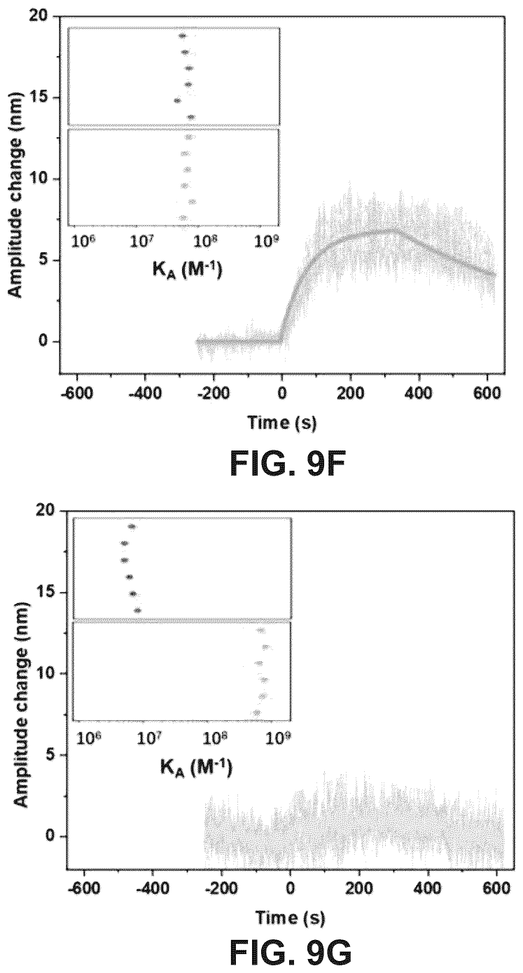

In the demonstration test depicted in FIG. 9A, around 350 nano-oscillators were assembled and simultaneously imaged on one sensor chip. The identification and classification of nano-oscillators to map the random distributions of arrays were achieved by examining the affinities of decoding hybridizations. To clearly illustrate the readout process, for each kind of virion bound nano-oscillator, six individuals nanocomplex were picked out and marked by specific colors in FIG. 6a. As shown in the FIGS. 9B-9G, the binding affinities of individual nano-oscillators to the two decoding hybridizations indicate the encoded states. The darker and lighter dots separately represent the affinity constants for each decoding hybridizations, and the distributions of these dots show the patterns defined by the combinations of barcoding sequences. Based on the encoded states, different GPCR-displayed nano-oscillators were successfully clustered. In FIGS. 9B-9G, with measurements of the six individuals in one cluster, the binding response curves (gray curves) of GPCRs specifically interacted with their respective target ligands were presented. The average effective responding curves for each kind of GPCRs were used to generate the corresponding first order kinetic fitting curves (solid curves with colors matching the star marks in FIG. 9A) for quantification of ligand-protein binding kinetics. Due to their inherent sensitivity to charge rather than molecular weight, individual nano-oscillators accurately measured interactions with small molecule targets. Notably, even molecules with a weight below 300 Da, such as propranolol (molecular weight=295.8 Da) in the ADRB2-propranolol pair illustrated in FIG. 9C, were precisely characterized. While, in FIG. 9G, nano-oscillators containing control virions showed negative responses to the introduced ligands. These results demonstrated the capability of the nano-oscillator array platform to characterize the binding kinetics of ligand-protein interactions in a multiplexing manner, particularly for small molecule ligands.

It is also worth pointing out that the limitations of the method. Firstly, the negatively charged DNA barcode may induce bias to the ligand binding kinetics for positively charged ligands due to the nonspecific electrostatic adsorption. Replacing DNA codes with less charged peptide nucleic acids (PNA) could address this issue. In addition, the array density is practically limited by the plasmonic propagation length, which generate parabolic pattern from individual nanocomplex that will overlap at high nano-oscillator density, as shown in FIG. 15. Feasible approaches to further improve the sensing throughput are: enlarge the field-of-view with a large camera size or lower zoom, reduce the plasmonic tails in the image experimentally or computationally, or eliminate the plasmonic tails and obtain diffraction limited images with plasmonic scattering imaging.

### Conclusion

In conclusion, we report a novel affinity encoded nano-oscillator platform that enables multiplexed, label-free quantification of small-molecule binding kinetics. The nano array is fabricated based on self-assembly of nano-oscillators on a gold coated sensor chip. Combining with the virion display technology, we show that the nanoarray can study membrane proteins in their native environment. The individual virion-AuNP conjugated nano-oscillator can be resolved with a prism coupled large-view plasmonic imaging system for real-time quantification of oscillation amplitude in a sub-nm precision. The electrical field driven, charge sensitive oscillation can detect molecular binding events induced charge changes, and particularly suitable for measure small molecule ligands binding kinetics to large membrane proteins. Using the same detection mechanism, the nanoarray is encoded with an affinity resolved DNA barcoding system that can provide significantly higher encoding states than conventional DNA barcoding with just a few decoding hybridizations. This encoding strategy can work for other type of samples attached to the gold nanoparticle, not limited to viral displayed membrane proteins, such as proteins, DNAs, membrane prep, nanodiscs encapsulated membrane proteins, etc. The gold nanoparticle could also be substituted by other type of nanoparticles, such as magnetic beads, polystyrene or silica nanoparticles, etc. As a multiplexed and addressable platform to study ligand-protein interaction, this nano-oscillator array sensor can quantify numerous binding affinities and kinetics reliably and simultaneously, holding great promise to develop practical high-throughput screening techniques for biochemical processes study and drug discovery.

### Supplementary Information

### 1. Materials.

SRIF-14 was purchased from Sigma-Aldrich. Neurotensin and Dynorphin A were purchased from AnaSpec, Inc. Propranolol hydrochloride was purchased from Bio-Techne Corporation. D1 antagonist was purchased from Hello Bio, Inc. Biotin-PEG-NHS (1000 Da), thiol-PEG-biotin (10 kDa), and thiol-PEG-NHS (10 kDa) were purchased from Nanocs Inc. Methoxy-(PEG)4-thiol was purchased from Thermo Fisher Scientific. 1×PBS was purchased from Corning. BK7 glass cover slip was purchase from VWR. Deionized (DI) water with conductivity of 18 MΩ·cm⁻¹ was used in all the experiments. DNA sequences were purchased from Integrated DNA Technologies, Inc. Their sequences are presented in Table 1.

TABLE 1
Sequences of labeled barcodes and decoding oligonucleotides
employed in this example.

| Name | Sequence | SEQ ID NO: |
|---|---|---|
| decoding | 5′-GAGAAGGGCCGAGGTATTGT-3′ | 1 |
| sequence 1 | | |
| barcode 1 | biotin-(CH₂)₆-5′-ACAATACCTCGGCCCTTCTC-3′ | 2 |
| (group 1) | | |
| (0 mismatch) | | |
| barcode 2 | biotin-(CH₂)₆-5′-ACACTACCTCGGCCCTCCTC-3′ | 3 |
| (group 1) | | |
| (2 mismatches) | | |
| barcode 3 | biotin-(CH₂)₆-5′-ACACTACCTCAGCCCTCCTC-3′ | 4 |
| (group1) | | |
| (3 mismatches) | | |
| decoding | 5′-ACGAAGGTCTGTAGCAACTC-3′ | 5 |
| sequence 2 | | |
| barcode 1 | biotin-(CH₂)₆-5′-GAGTTGCTACAGACCTTCGT-3′ | 6 |
| (group 2) | | |
| (0 mismatch) | | |
| barcode 2 | biotin-(CH₂)₆-5′-GAGTTACTACAGACCATCGT-3′ | 7 |
| (group 2) | | |
| (2 mismatches) | | |
| barcode 3 | biotin-(CH₂)₆-5′-ACACTACCTCAGCCCTCCTC-3′ | 8 |
| (group 2) | | |
| (3 mismatches) | | |

### 2. Sample Preparation.

BK7 cover slips were rinsed by acetone and DI water and then dried by nitrogen flow. Gold sensor chip was fabricated by a thermal evaporator (Auto306, Edwards) with 2 nm chromium and 47 nm gold on the cleaned cover slips. The gold chip was then immersed overnight in a solution containing biotin-(PEG)n-thiol (10 KDa) and methoxy-(PEG)4-thiol at a ratio of 1:100. This resulted in the assembly of biotin linker PEG with a long length and spacer PEG with a short length on the gold chip.

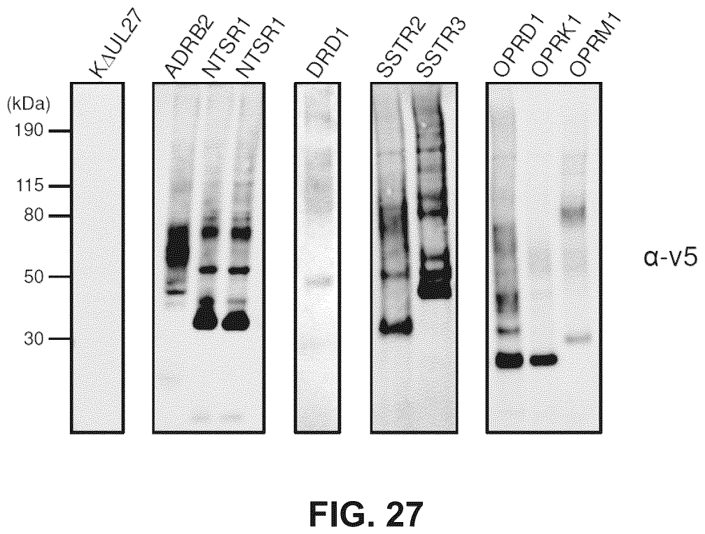

Human GPCR-displayed virions were prepared according to previously published paper. FIG. 27 shows the Western blot result of the GPCR-displayed virions. The virions were first incubated with biotin-(PEG)n-NHS (1000 Da) at a 1:2 ratio in 1×PBS for 1 hour to form a biotin-(PEG)n modified virion structure. As shown in FIG. S2a, the virions were then incubated with streptavidin-coated AuNPs at 1:1 ratio in 1×PBS for 30 minutes to obtain virion-AuNP nanocomplex through streptavidin-biotin binding. The dimerization process is governed by Derjaguin-Landau-Verwey-Overbeek (DLVO) theory, which predicts that when two particles with similar diameters (greater than 100 nm) are in close proximity, chemical bonding can occur.

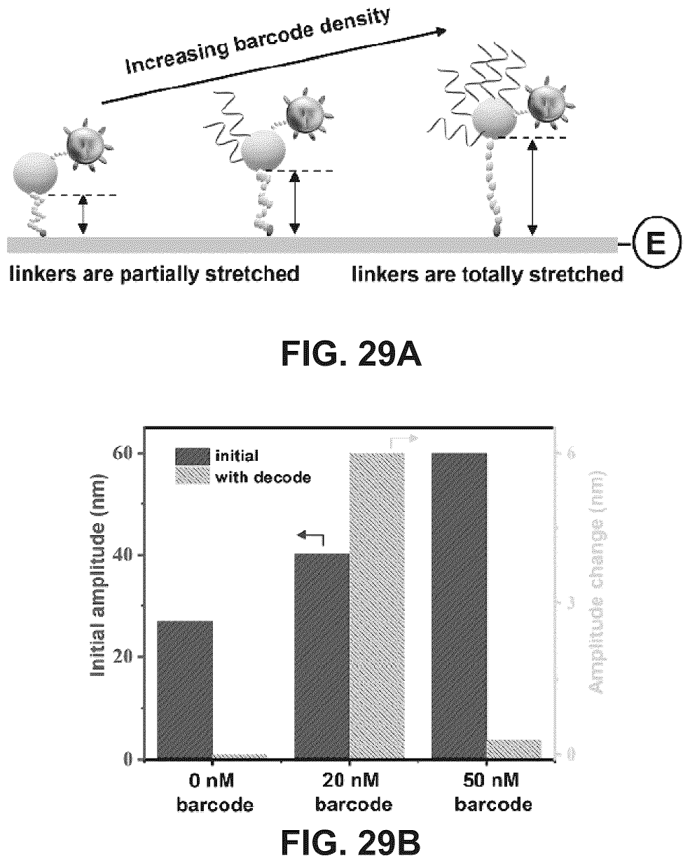

In the proof-of-concept study, to specifically encode nanocomplexes of different GPCR virions, a combination of two groups of biotin-labeled DNA barcode sequences with a final concentration of 20 nM, was incubated for an additional 30 minutes. This concentration was selected to effectively modify the nano-oscillators while preventing signal saturation during decoding, as illustrated in FIG. 29. Following this, two rounds of centrifugation at 5000 rpm for 10 min were performed to collect the deposit. Subsequently, the deposit was dispersed by PBS buffer to obtain a batch of DNA-coded virion-gold nanoparticle nanocomplexes. One type of virion was encoded per batch. The library was constructed by mixing different batches (FIG. 28b). The barcode combinations for the specific virion have been list in Table 2.

Finally, the nanocomplexes were diluted into a concentration about 10⁴/μL and added onto the prepared gold sensor chip with PEG modification to form the self-assembled oscillators. The chip was incubated with the nanocomplexes for 30 minutes to allow the streptavidin coating on the nanocomplexes to bind to the biotin moieties on the linker. The high spacer/link ratio (100:1) was employed to control the linker density and minimize non-specific adsorption. A silicone liquid cell was cleaned and mounted on the gold sensor chip for handling the solution and flow.

TABLE 2
Barcode combinations for addressing the specific virions.

| Target small | Virion | |
|---|---|---|
| molecule | in the | |
| Ligand | nanocomplex | Barcode combination |
| Neurotensin | NTSR1 | barcode 1 (group 1) and barcode 1 (group 2) |
| DynorphinA | OPRM1 | barcode 1 (group 1) and barcode 2 (group 2) |
| Propranolol | ADRB2 | barcode 1 (group 1) and barcode 3 (group 2) |
| D1 | DRD1 | barcode 2 (group 1) and barcode 1 (group 2) |
| antagonist | | |
| SRIF14 | SSTR2 | barcode 2 (group 1) and barcode 2 (group 2) |
| N/A | K082 | barcode 3 (group 1) and barcode 1 (group 2) |

### 3. Experimental Setup.

The plasmonic imaging measurement were conducted using a commercial prism-based SPR imaging system (SPRm200, Biosensing Instrument Inc., Tempe, Arizona, USA) with a 690 nm, 1 mW laser (illumination area ˜1.2 mm×0.8 mm), and a custom installed USB3 CMOS camera (MQ003MG-CM, XIMEA, Germany). The effective field of view is determined by 330 μm×250 μm. A sinusoidal potential was applied to the gold chip with a potentiostat (AFCBP1, Pine Instrument Company) and a function generator (33521A, Agilent) using the standard three-electrode setup, where gold sensor surface, platinum wire, and Ag/AgCl serve as working, counter and reference electrode. Unless indicated otherwise, a sinusoidal potential with amplitude, 350 mV, and frequency, 5 Hz, was applied in the measurements. A USB data acquisition card (NI USB-6251, National Instruments) was used to record the time stamp of the images from the camera in order to synchronize plasmonic imaging with electrical measurement. Optical imaging sequence was recorded at 50 frames per second (FPS) simultaneously to capture the oscillation processes.

### 4. Measurement Workflow.

The first step of the measurement is target screening, which quantifies the binding kinetics of all ligands to the GPCRs on the nanoarray sequentially. After a steady baseline was achieved with the running buffer, a target small molecule ligand was introduced for 300 seconds to measure the association of the ligand to the GPCRs. Next, the flow was switched back to buffer to measure the dissociation of the ligand from the GPCRs for a duration of 250 seconds. All ligands were measured sequentially on the same nanoarray following this protocol.

The second step of the measurement is decoding, which identifies the type of GPCRs on individual nano-oscillators, and was achieved by sequentially measuring the binding affinity of all decoding sequences. Each round of decoding flowed 50 nM of a specific readout sequence to hybridize with the barcodes on the nano-oscillator for 550 seconds, followed by buffer washing to dissociate the readout sequence for a duration of 250 seconds. Conducting the ligand screening before decoding can prevent potential issue of saturated oscillation amplitude change that may arise from excessively high negatively charged nano-oscillators after hybridization with the decoding sequence. Throughout all experiments, the injected sample was driven by a syringe pump and flowed onto the upper right portion of the field-of-view to minimize mass transport issues.

### 5. Data Processing.

After recording the images, a region of interest (ROI) was selected on each particle (covering the parabolic tail), and the mean intensity within the ROI was calculated as the plasmonic intensity. An adjacent region with the same size was selected as the reference region, and its intensity was used to remove common noise from the ROI. The oscillation amplitude was obtained in every second using fast Fourier transform (FFT). The FFT intensity at the oscillation frequency was then plotted against time to illustrate both the binding trend and decoding process. Response curve fitting and calculation of binding kinetic constants were conducted using Scrubber (BioLogic Software). All data were visualized using Origin and MATLAB.

### 6. Nano-Oscillator Characterization.

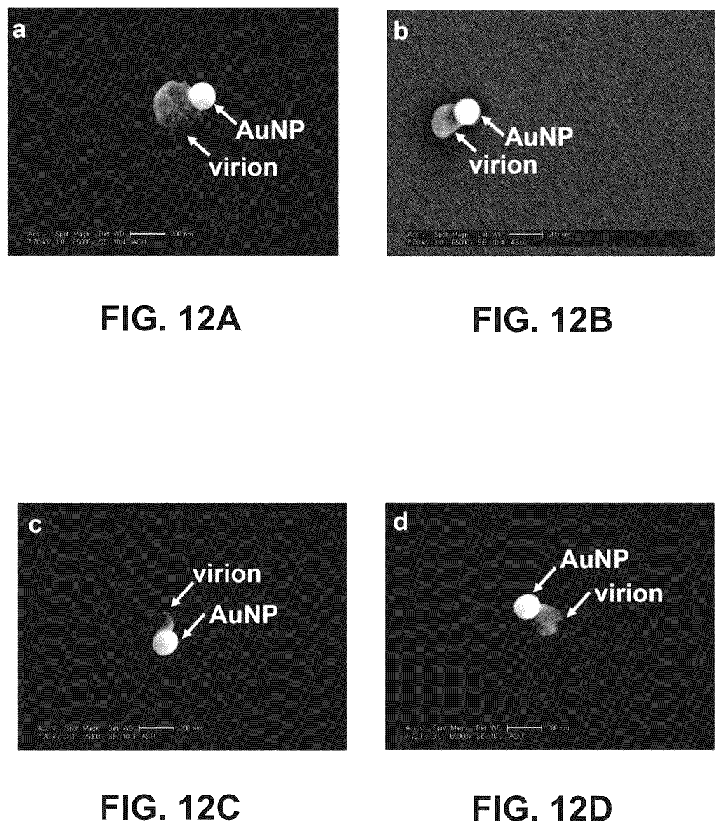

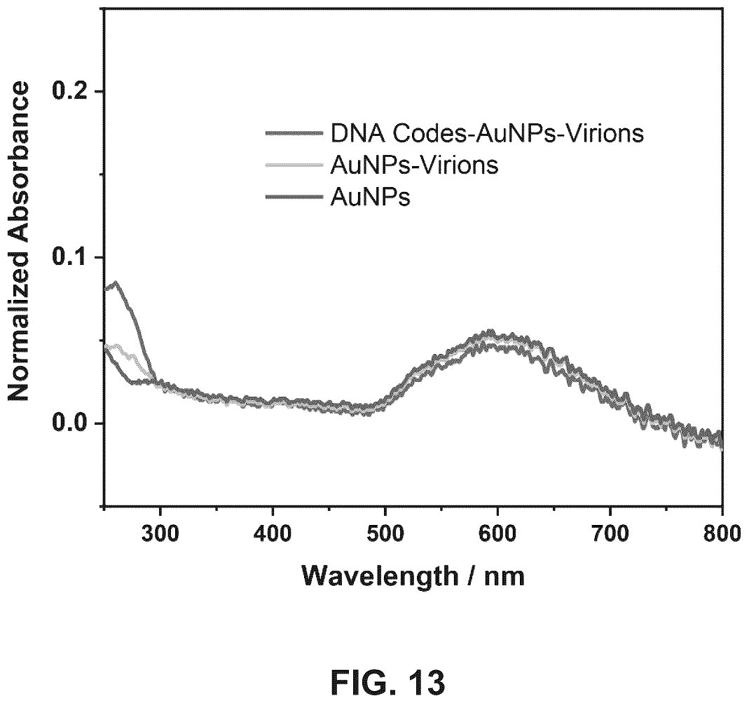

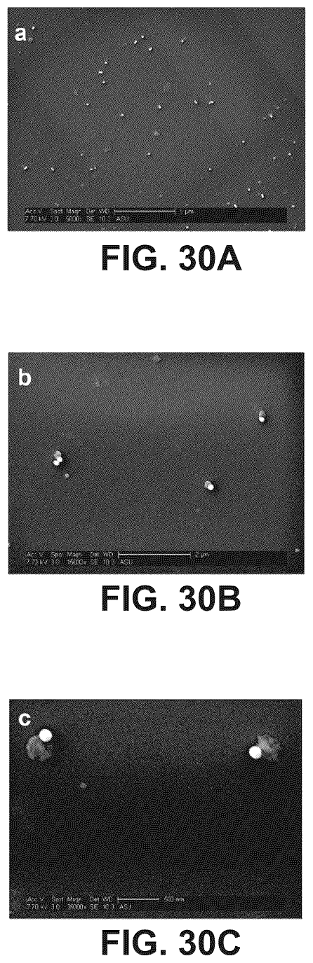

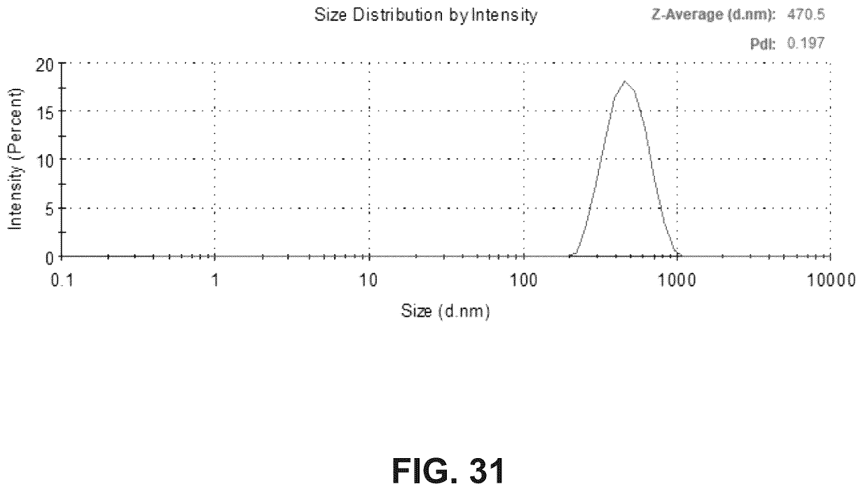

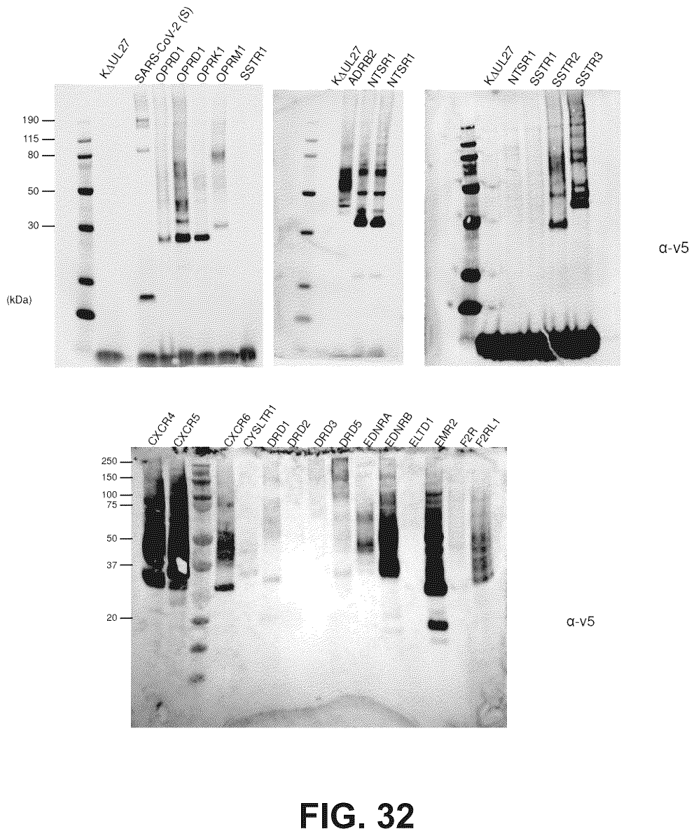

FIG. 10 illustrates a time sequence of SPR images for monitoring nano-oscillators assembled on the gold chip. Three representative nano-oscillators are highlighted with arrows in FIG. 10A upon they assembled on the sensor surface. The SPR intensity changes of the ROIs (as marked in S1a) where these nano-oscillators assembled on the gold chip are plotted in FIG. 10B, and the SPR signal intensities increased in the range of 150-200 mDeg upon assembly of the nano-oscillators. Similarly, tracking the SPR intensities of separate virions and AuNPs attached to the gold chip, statistical results shown in FIG. 11 demonstrate that nano-oscillators comprising virion-AuNP complexes present the maximum signal intensity. As depicted in FIGS. 12 and 30, further examination of the nano-oscillator structure through scanning electron microscopy confirms the successful connection between the virion and the gold nanoparticle. Additionally, as shown in FIG. 13, UV-vis spectra were employed to qualitatively investigate the connection with virions, where the lightest greyscale curve represents absorption at 280 nm for proteins, and DNA barcodes are indicated by the medium shade greyscale curve with absorption at 260 nm for DNA. As shown in FIG. 31, a single hydrodynamic size peak measured by dynamic light scattering, along with a low polydispersity index, indicates the narrow size distribution of the AuNp-virion nanocomplex.

### 7. Control and Validation Experiments.

To confirm that the observed amplitude changes of the nano-oscillator were attributed to ligand-GPCR interactions rather than interference from the virion envelope or adjacent AuNPs with DNA barcodes, we tested nano-oscillators fabricated using K082 virions with no GPCR displayed on the envelope, and nano-oscillators with AuNPs only without virion. As depicted in FIG. 14, no discernible responses to ligands were observed, suggesting that the oscillator structure itself did not yield false positive responses.

In addition, we compared ensemble SPR oscillation measurements to single oscillator detection. FIG. 15 shows SPR image of high density of virion oscillators spotted on the substrate, which created an oscillating area that provide ensemble responses to ligand binding. The dose-dependent measurement results from the ensemble virions were found to be consistent with the single nano-oscillator measurement results, indicating that this nano-oscillator design can accurately measure the GPCRs.

Decoding responses were examined in FIG. 17, where obvious amplitude changes upon introducing and washing of the decoding sequence were observed. The response curves were fitted with first order kinetic model to obtain distinct binding affinities among different groups of barcodes, confirming that this DNA hybridization affinity based decoding strategy is working at the single nano-oscillator level. Table 3 presents the statistics of group 1 and group 2 barcodes hybridization signal amplitude and standard deviations, as well as predicted number of individual nano-oscillators needed to reach 95% confidence level. The statistics of GPCRs displayed oscillators responding to their corresponding ligand pairs were also listed in Table 3.

TABLE 3
Statistics of sensor experiments, and calculated number of
particles needed for 95% confidence in target detection.

| | Median of | | Calculated minimal |
|---|---|---|---|
| | amplitude | | number of nano- |
| | change for | | oscillators needed per |
| | association | S.D. | test to reach 95% |
| Particle group | (nm) | (nm) | confidence level |
| barcode 1 (group1) | 9.7 | 2.6 | 1 |
| (0 mismatch) | | | |
| barcode 2 (group1) | 9.6 | 3 | 1 |
| (2 mismatches) | | | |
| barcode 3 (group1) | 7.5 | 2.5 | 2 |
| (3 mismatches) | | | |
| barcode 1 (group2) | 11.6 | 4.9 | 2 |
| (0 mismatch) | | | |
| barcode 2 (group2) | 10.9 | 5.6 | 3 |
| (2 mismatches) | | | |
| barcode 3 (group2) | 7.2 | 2.9 | 3 |
| (3 mismatches) | | | |
| NTSR1 | 11.5 | 5.9 | 3 |
| OPRM1 | 10.5 | 4.5 | 2 |
| DRD1 | 7.8 | 1.3 | 2 |
| ADRB2 | 5.4 | 2.1 | 3 |
| SSTR2 | 6.4 | 2.6 | 3 |
| barcode 1 (group1) | 9.7 | 2.6 | 1 |
| (0 mismatch) | | | |
| barcode 2 (group1) | 9.6 | 3 | 1 |
| (2 mismatches) | | | |
| barcode 3 (group1) | 7.5 | 2.5 | 2 |
| (3 mismatches) | | | |
| barcode 1 (group2) | 11.6 | 4.9 | 2 |
| (0 mismatch) | | | |
| barcode 2 (group2) | 10.9 | 5.6 | 3 |
| (2 mismatches) | | | |
| barcode 3 (group2) | 7.2 | 2.9 | 3 |
| (3 mismatches) | | | |
| NTSR1 | 11.5 | 5.9 | 3 |
| OPRM1 | 10.5 | 4.5 | 2 |
| DRD1 | 7.8 | 1.3 | 2 |
| ADRB2 | 5.4 | 2.1 | 3 |
| SSTR2 | 6.4 | 2.6 | 3 |

### Example 2: Spotting-Free Nano-Oscillator Array for Quantification of Virion Displayed Membrane Protein Binding Kinetics

### Introduction

Membrane proteins are the most popular drug targets, accounting for over a half of the FDA approved drugs. A key task in studying the physiological functions and developing drugs is to measure the kinetics of ligand binding to membrane protein targets. Unfortunately, such measurements remain extremely challenging, particularly for transmembrane receptors such as G-protein coupled receptors (GPCRs), which require the native cellular membrane to preserve conformation and normal function. To further exacerbate the problem, small molecules are the most preferred form for therapeutic drugs, comprising most of the currently available drugs. However, small molecule detection is difficult, and no techniques exist that enable direct high throughput kinetic measurement of small molecule interactions to membrane proteins in their native conformations.

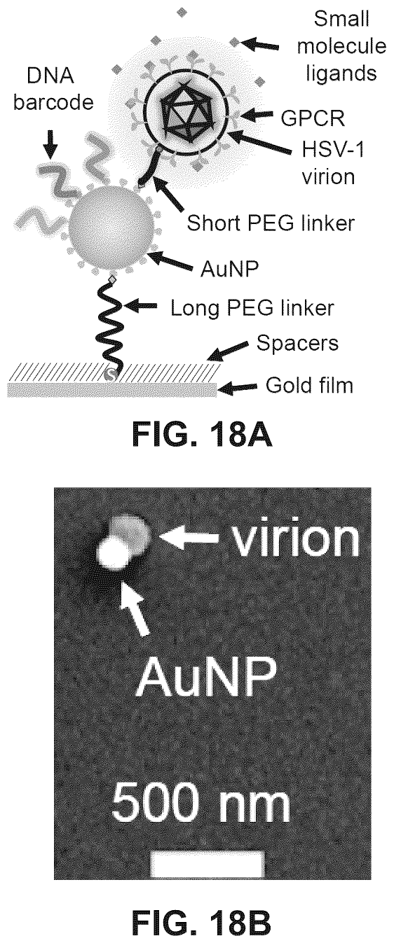

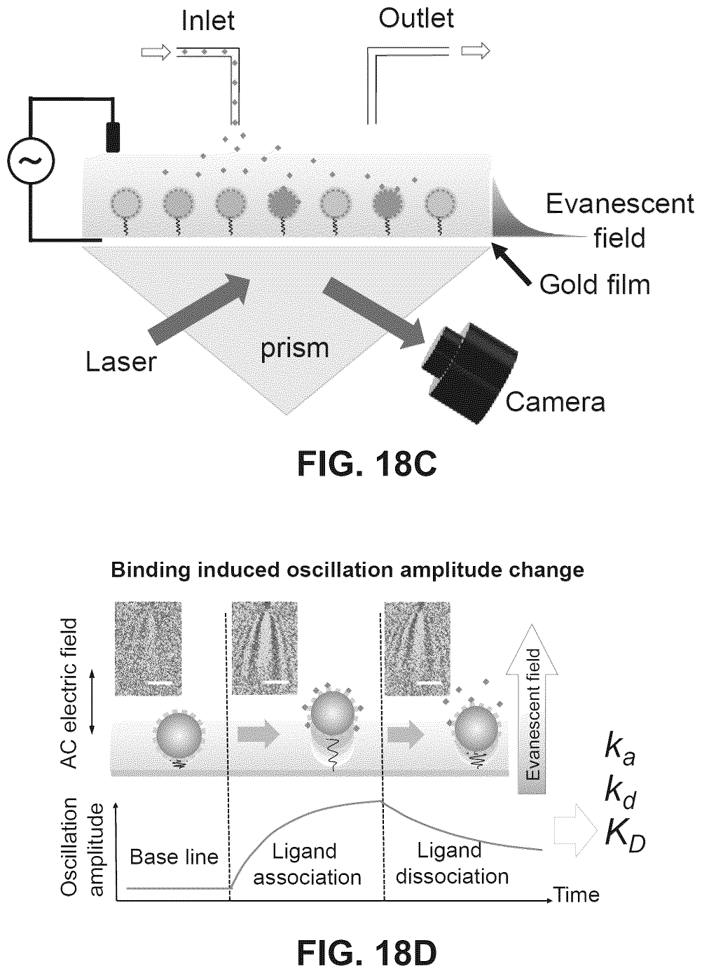

To overcome these challenges, a spotting-free nano-oscillator array (SFNOA) technology is presented in this example. SFNOA enables high throughput quantification of small molecule ligand binding kinetics to virion-displayed transmembrane proteins (VirD). Human GPCRs displayed on the human herpes simplex virus-1 (HSV-1) envelope are measured to showcase the capability of the SFNOA technology. The HSV-1 envelope is derived from the human cellular Golgi membrane, and thus preserves the native conformations and post-translational modifications of the transmembrane proteins. As shown in FIG. 18, SFNOA uses a random nano-oscillator array consisting of different types of VirD membrane proteins and quantifies the binding kinetics of large and small molecule ligands to individual oscillators via an innovative charge sensitive plasmonic imaging technique. The individual nano-oscillators in the array are addressable with a high efficiency affinity encoded DNA barcoding method, which enables spotting-free fabrication and rapid read-out of thousands of nano-oscillators in parallel for high throughput multiplexed measurement.

This technology removes the tedious steps required to extract and purify multi-pass transmembrane proteins, overcomes instability issues associated with protein reconstitution, preserves native conformation and function of membrane proteins, and performs high throughput kinetic quantification of both large and small molecule ligand. To highlight the significance of SFNOA, a large VirD collection of 315 non-odorant human GPCRs, which cover 85% of all annotated non-odorant GPCRs in humans and many of them are the preferred drug targets, are utilized to demonstrate high throughput measurement of small molecule, peptide and protein ligands binding kinetics to these membrane proteins.

### Membrane Proteins, Including GPCRs, are the Most Popular Drug Targets

Membrane proteins relay signals between a cell and its external environment, transport ions and molecules in and out of the cell, and allow cells to recognize and interact with other cells. They are also the most popular drug targets, accounting for over a half of the FDA approved drugs. A particularly important family of human transmembrane proteins is G-protein-coupled receptors (GPCRs), which constitute the largest and most diverse transmembrane protein family in the human genome with over 800 members identified to date. GPCRs play critical roles in numerous cellular and physiological processes, including cell proliferation, differentiation, neurotransmission, development and apoptosis, and cancer growth and development. Unsurprisingly, over 30% of FDA-approved drugs target this family. For example, GPCRs are considered to be the most useful drug targets of many solid cancers. Tumor cell proliferation is regulated by many neuropeptides, and signaling molecules, such as prostaglandin E2, thrombin, sphingosine-1-phosphate, lipoprotein A and interleukin 8, which often target GPCRs to initiate the downstream signaling networks that involve second messenger-generating systems, small GTPases of the Ras and Rho families, and MAPK cascades. A recent review listed more than 47 relevant non-odorant GPCRs in 13 different cancers.

Challenges in Studying Transmembrane Proteins in their Native Membrane Environment

Understanding the vital cellular functions and screening new drugs targeting membrane proteins require accurate measurement of ligand drug binding interactions with membrane proteins. Despite the importance, developing such a capability has been challenging. Traditional methods use radiolabelled or fluorescent-labelled ligands. These technologies are end-point measurements, which provide affinity but not binding kinetics. Binding kinetics is critical for determining drug efficacy, residence time and biased agonism, and for elucidating ligand-target binding mechanism in drug design. To determine molecular binding kinetics, label-free detection technologies have been developed. However, these technologies usually require extracting proteins from cells, followed by purifying the extracted proteins and immobilizing them on a sensor surface for binding measurement. This approach unfortunately cannot be directly applied to GPCRs and other multi-pass transmembrane proteins as these proteins unavoidably lose their native conformations and functions outside of their membranes.

Several methods have been developed to prepare and stabilize transmembrane proteins for binding kinetic studies. Examples include inserting the proteins into artificial membrane bilayers, such as lipid vesicles and nanodisc, and cell membrane preps. We have developed a method to directly measure membrane protein binding kinetics on whole cells. Despite the success, high throughput quantification of ligand or drug binding with a large collection of membrane proteins remains challenging. This is especially the case for measuring small molecule binding to the proteins. Small molecules are difficult to detect and quantify because existing label-free detection technologies typically detect relative changes in mass, which is miniscule for small molecules. Despite this obstacle and at great cost, small molecules remain the predominant drug format for transmembrane proteins, and account for ˜90% of the FDA approved drugs. Indeed, a capability for high throughput quantification of small molecule kinetic interactions to transmembrane proteins in their native conformation is thus an important need to advance membrane protein study and develop new drugs targeting transmembrane proteins. No pre-existing method meets these needs.

Spotting-Free Nano-Oscillator Array with Virion Displayed Membrane Proteins

To address this unmet need, this example illustrates a spotting-free nano-oscillator array (SFNOA) detection technology that can directly measure binding kinetics of virion displayed (VirD) multi-pass membrane proteins. As illustrated in FIG. 18, SFNOA consists of a random nano-oscillator array that is individually addressable via an innovative affinity encoded DNA barcoding system, and uses a charge sensitive nano-oscillator plasmonic detection platform to achieve label-free and real-time kinetic measurements of binding between small molecule ligands and membrane proteins. GPCRs are displayed in HSV-1 envelopes derived from human Golgi membranes, which preserve the native conformations and post-translational modifications of the GPCRs. Each VirD-GPCR is conjugated to a gold nanoparticle (AuNP), which is tethered to a plasmonic sensor chip (gold-coated glass slide) via flexible PEG linkers to form a nano-oscillator. By applying an alternating electric field to the gold surface, the nano-oscillator is driven into oscillation and its oscillation amplitude is monitored using a plasmonic imaging technique with sub-nm precision. When ligand molecules bind to the GPCRs on the nano-oscillator, the oscillation amplitude changes, which is measured in real-time to provide precise binding kinetics. The AuNP not only amplifies the plasmonic signal by 3× and thus the detection sensitivity, but it also facilitates multi-state encoding with DNA barcodes for efficient read-out (more details are provided herein).

VirD: A Virion Display Technology for Displaying Membrane Proteins in their Native Membranes

A VirD technology for profiling functional membrane proteins has been developed by others. VirD uses recombinant HSV-1 to display human GPCRs on the viral envelopes by replacing a gene expressing the major viral envelope glycoprotein B (gB) with a GPCR ORF. The HSV-1 envelope is derived from the human Golgi membranes to preserve the native conformations and post-translational modifications of the membrane proteins. The VirD technology is non-virulent, and eliminates the need of extraction, purification, and reconstitution of the membrane proteins, which alleviates the aforementioned difficulties in the existing methods. VirD has been developed for 315 non-odorant human GPCRs, which covers 85% of all annotated non-odorant human GPCRs as preferred drug targets. The remaining critical challenge is to measure the affinity and kinetics of ligand molecules and drug candidates binding with the GPCRs in a high throughput fashion. Each virion has a large mass (˜200 MDa) compared to a protein (˜100 kDa) and therefore, the relative mass change caused by binding to a protein ligand is small. The relative mass change associated with the binding of a small molecule ligand (˜100-1k Da) is even smaller. Currently, no commercial technologies can detect such small mass changes in the virions in real time.

### Nano-Oscillators: A Label-Free Technology for Quantifying the Binding of Large and Small Ligands to Transmembrane Protein on Single Virions

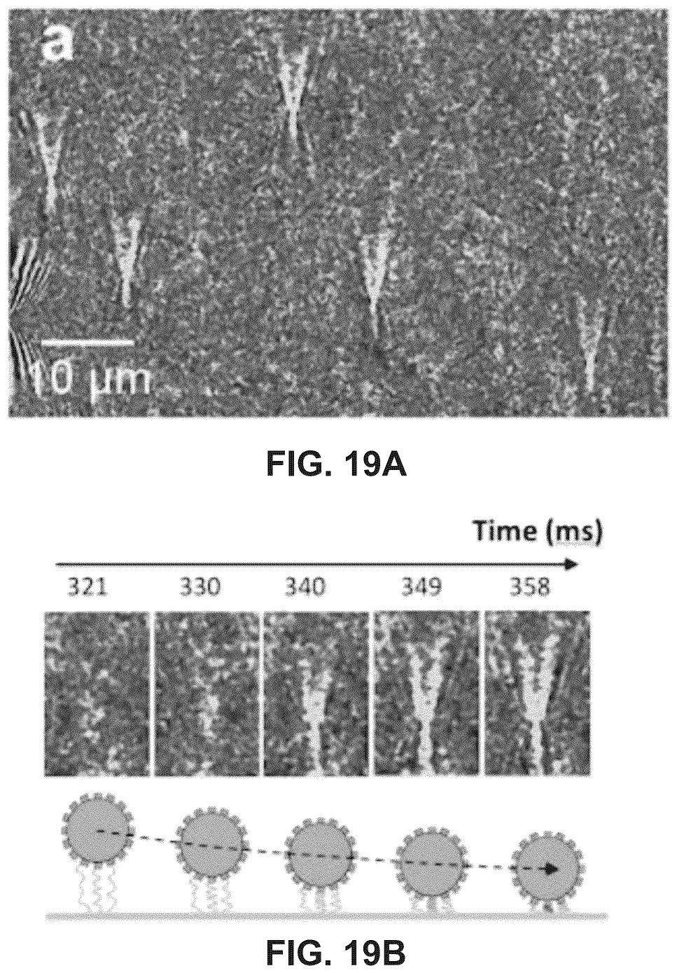

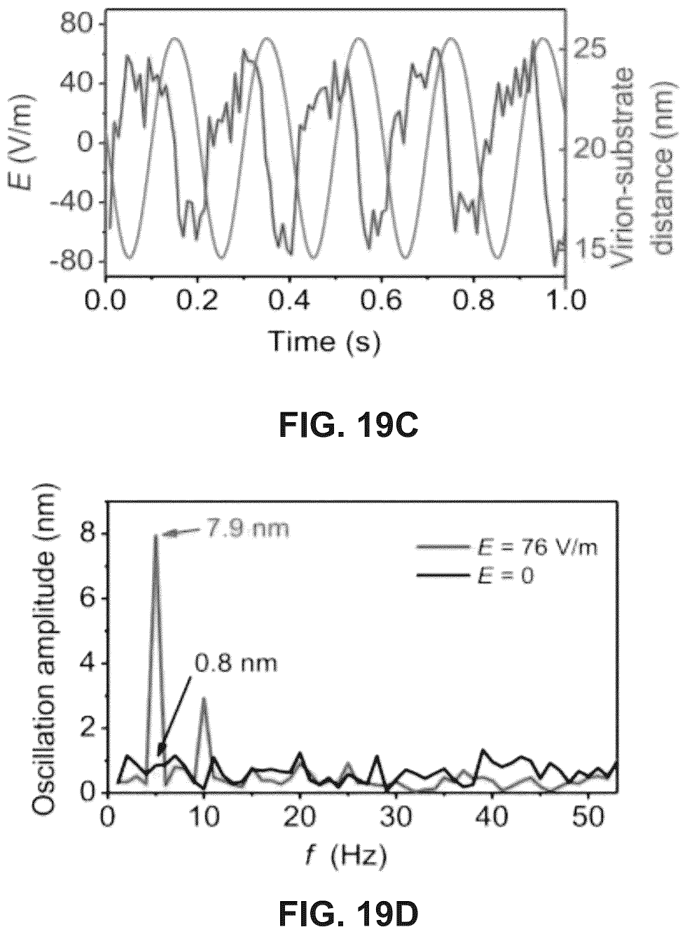

We have developed a label-free nano-oscillator detection technology, which features synergistic coupling of the VirD technology with an innovative plasmonic imaging technology for sensitive quantification of large and small molecule binding kinetics to transmembrane proteins while in their native conformation. Each VirD is tethered to the gold sensor chip via a flexible polyethylene glycol (PEG) polymer linker (FIG. 19B). The VirD is driven into oscillation with an alternating electric field, and the oscillation amplitude (z₀) is given by

$${z}_{0} = \frac{q *}{k *} {E}_{0} , \qquad ( 1 )$$

where q* is the effective charge of the oscillator, k* is the effective spring constant of the polymer linker, and E₀ is the amplitude of the alternating electric field. Upon ligand binding to the target proteins on the oscillator, a change in the charge profile occurs resulting in a change in oscillation amplitude (FIG. 18D). Fortunately, most biologically relevant ligands and drugs are charged or partially charged (containing charged groups), which accounts for 99.5% of the 2039 FDA approved small molecule drugs. Uncharged ligands can still alter the charge profile via conformation changes upon binding to the membrane proteins, thus producing detectable changes in oscillation amplitude. To confirm this, we have shown that both charged and uncharged molecules can be detected (see data provided herein). Since the nano-oscillator detection technology is not based on mass detection, its sensitivity does not diminish with the molecular mass of the ligand, making it suitable for studying both large and small ligands. E₀ and k* in Eq. 1 can be determined with calibration, from which the effective charge (q*) can be quantified. It is important to note that for binding kinetics measurement, only the relative change in the oscillation amplitude vs. time needs to be measured. For this reason, the number of the linkers to each nano-oscillator only affects k*, having no direct effect on the binding kinetics.

### Sensitive Detection of Single Virion Oscillation Via Plasmonic Imaging

The detection limit of the nano-oscillator is determined by how accurate one can measure the oscillation amplitude (Eq. 1). Using a plasmonic imaging system, we have shown that the oscillation amplitude of a nano-oscillator can be detected with sub-nm precision (FIG. 19). This extremely high sensitivity comes from the scattering of the plasmonic wave by the nanoscale oscillator, which produces a parabolic pattern for each oscillator (FIG. 19A) with image intensity that decays rapidly with the distance between the oscillator and the chip surface (FIG. 19B). The plasmonic imaging (parabolic pattern) intensity (I) is related to the distance between the oscillator and the sensor surface (z), given by

$$Δ I / I = - {z}_{0} / 100 nm \qquad ( 2 )$$

where ΔI/I is the relative intensity change of the oscillator, and 100 nm is the decay distance of the evanescent field. Our experiments have shown that the oscillation can be tracked in real time (FIG. 19C) with a precision of ˜0.8 nm (FIG. 19D). The corresponding charge detection limit is ˜1.4e, where e=1.6×10⁻¹⁹ C, the charge of a single electron. This detection limitation is among the most sensitive detection technologies.

### Affinity Encoded SFNOA for High Throughput and Multiplexed Readout

Traditional microarray spotters produce spots >100 μm in diameter, which is far too large for the fabrication needs of >1000 spot nanoarray that can be detected with high resolution real time imaging. Serial assembly of different biofunctionalized nanoparticles onto a chip while continuously recording their adsorption positions has been attempted, but the slow batch to batch adsorption for nanoarray formation reduces the throughput and screening efficiency of large biochemical libraries, thereby limiting its commercial feasibility.

In this example, we present an innovative DNA barcoding strategy using affinity encoded, high efficiency multi-state decoding for spotting-free one-step fabrication of individually addressable random nano-oscillator arrays. To address individual nano-oscillators in the array, we developed an encoding-decoding strategy using DNA sequence specific binding affinities. The encoding protocol is detailed further herein. Briefly, a streptavidin-coated AuNP core is implemented to both enhance the plasmonic signal for sensitive detection and establish a centralized docking surface for attaching the VirD-GPCR and biotinylated DNA barcodes. In this way, a library of DNA encoded nano-oscillators containing different GPCRs can be readily generated (FIG. 18A). To form a multiplexed nanoarray, various nano-oscillators are simply mixed together and self-assembled to a sensor surface pre-functionalized with biotinylated PEG linkers in one step, without using any array spotter (FIG. 18C).

As shown in FIG. 18D, the assembled SFNOA is first exposed to buffer to establish a stable baseline, then exposed to a ligand to measure the association, followed by flowing buffer again to measure the dissociation, and the whole process is recorded by plasmonic imaging. The unique responses from each individual nano-oscillator are extracted from the image sequences to obtain the binding kinetics of each oscillator to the ligand.

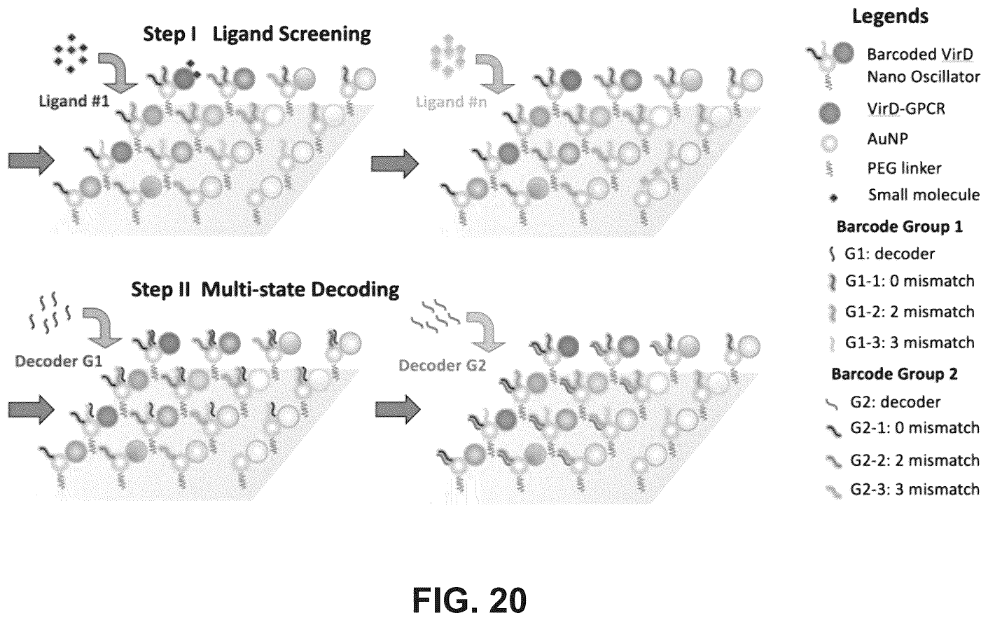

As shown in FIG. 20, multiple ligands can be measured sequentially on the same chip. After the binding kinetics of each nano-oscillator to each drug has been measured, decoding can occur. The decoding processes is performed by hybridizing the decoding sequences to the barcodes of the nano-oscillators and then consequently measure the hybridization affinity. Same as measuring the ligand binding kinetics, DNA hybridization changes the charge profile of the individual oscillators that are quantified by the changes of their oscillation amplitudes.

To expand the addressability of the DNA barcoding scheme, the barcodes are divided into decoding groups by a distinct decoding sequence. In each decoding group, barcodes have sequences that are absent, complementary, or mismatched to varying degrees to the decoding sequence, thereby permitting for distinct hybridization affinities corresponding to each barcode sequence. In this way, the decoding time is significantly expedited, whereby each member of a decoding group is decoded by just one hybridization of a single decoding sequence, which produces distinct hybridization affinities that directly correspond to the number of mismatches in the hybridization.

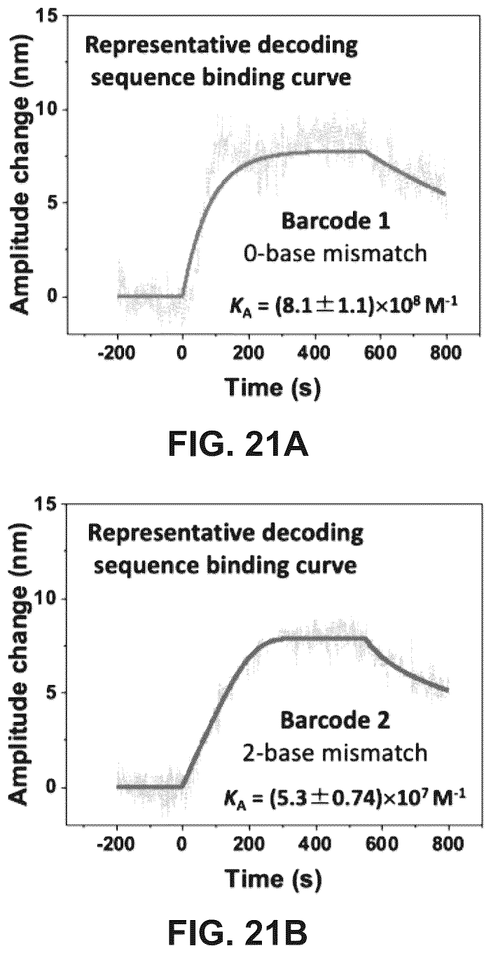

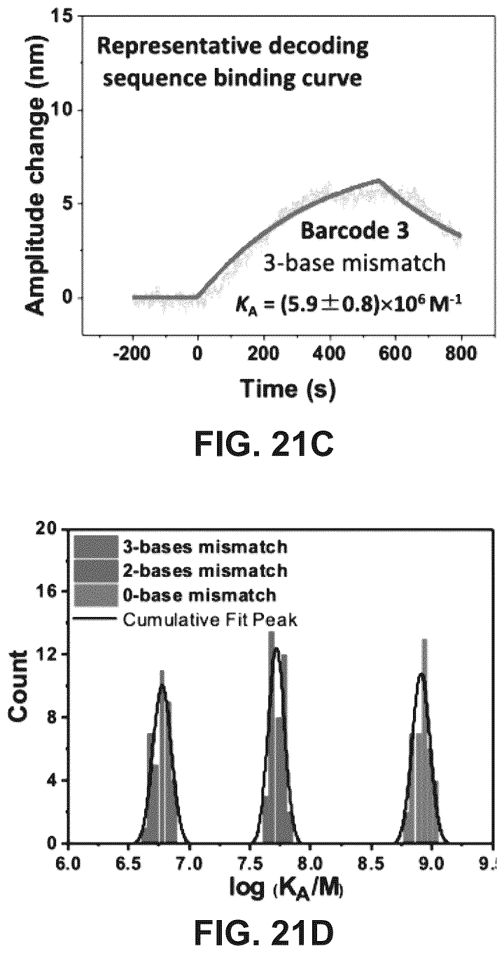

In this way, for each decoding group containing j types of DNA barcodes, j+1 states can be encoded, since “no barcode” is also an encoding state. If the number of coding groups is m, then the combinatorial assignment of DNA barcodes from different decoding groups would generate an impressive (j+1)^{m} distinct states, with each nano-oscillator only needing to be labelled with m types of barcodes. In contrast, a conventional DNA barcoding strategy is binary, where only the “with” and “without” barcode states are observed, producing merely 2^{m} distinct states. The data in FIG. 21 shows that by mismatching up to 3 DNA bases, 3 distinct hybridization affinities can be resolved to produce (3+1) robust states (no barcode, 0, 2 and 3 mismatches). Therefore, for m groups of decoding sequences, 4^{m} distinct states can be read, which is 2^{m} times more than the conventional DNA barcoding method. This innovative barcoding approach exponentially scales the addressing capacity so that large libraries can be readily encoded with only a few decoding groups. Conservatively, based upon this preliminary data, only 5 decoding groups, producing 4⁵=1024 distinct states, would be sufficient to cover the entire 315 non-odorant human VirD-GPCR collection. In comparison, the conventional DNA barcoding method would require 9 barcodes and decoding sequences to generate the 2⁹=512 distinct states necessary to address the same sample collection.

This novel approach to DNA-based encoding is conveniently read-out in high throughput on the SFNOA platform using plasmonic oscillations and provides a powerful tool to efficiently address large VirD libraries of nano-oscillators.

### Exemplary Key Features and Significance of SFNOA and Detection Platform

The SFNOA technology disclosed herein has the following innovative features:
Virion display of transmembrane proteins removes the tedious protein extraction, purification, and reconstitution steps, and preserves the native functions of the transmembrane proteins.
VirD provides a large collection of important GPCRs that are readily accessible and mass-producible.
Charge sensitive nano-oscillators enable quantification of small molecule ligand binding kinetics.
Plasmonic detection allows label-free, real-time, and high throughput quantification of large and small molecule binding kinetics.
High efficiency manufacturing of barcoded nano-oscillators in a spotting-free single step fabrication.
Affinity encoded DNA barcodes for high efficiency multi-state decoding with 4^{m} multiplexing using only m decoding sequences.

These exemplary features enable a more biologically relevant study of molecular interactions of GPCRs and other transmembrane proteins associated with diseases, and accelerate drug development by providing quantitative binding constants (for accurate ranking of drug candidates).

### Results

### Production of 315 Non-Odorant VirD GPCRs

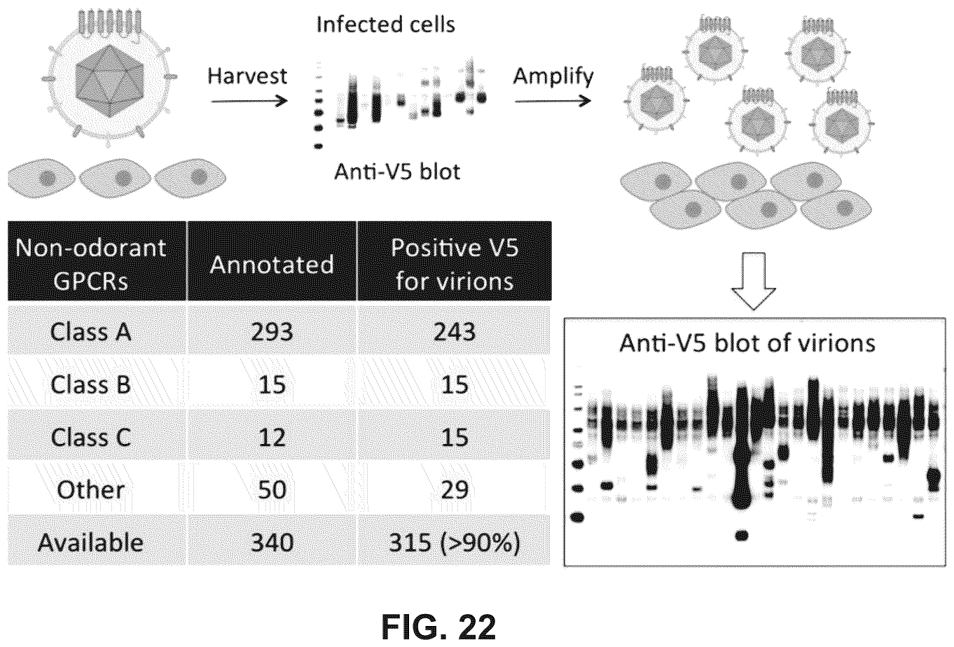

The human non-odorant GPCRs were cloned into the HSV-1 genome using the Gateway (Life, USA) method. As shown in FIG. 22, each GPCR was expressed as a viral gene under the control of the UL27 gene (the gene of gB) promoter, and the expressed polypeptide contains a C-terminal V5 epitope tag. To produce virions incorporating GPCRs, we infected Vero cells (3×10⁷ cells) at a multiplicity of infection of five plaque forming units/cell. The extracellular medium of these infected cells was collected 48 hours after infection, and then purified and concentrated via centrifugation through a 20% sucrose cushion. The final virion pellets were re-suspended in 30% glycerol and stored at −80° C. before use. Incorporation of the GPCR molecules in the virion was confirmed by immunoblot analysis using anti-V5 antibody. Of the 340 available non-odorant human GPCRs in our collection, 92% of them showed detectable anti-V5 signals. The function of the 315 VirD-GPCRs displayed in the virions were also validated by 20 monoclonal antibodies (mAbs) raised against the ectodomains of human GPCRs with a fluorescent microarray.

### Fabrication of VirD Oscillators

We fabricated VirD oscillators by tethering single HSV-1 virions (˜200 nm in diameter) to a gold sensor chip via PEG linker (63 nm in length) (FIG. 19B). The PEG has a thiol group on one end to bind to the gold surface, and a N-hydroxysuccinimide (NHS) moiety on the other end to form a covalent bond to the virions via the primary amines of the viral glycoproteins in the envelopes. The virions were incubated with HS-PEG-NHS at 1:5 ratio in 1×PBS overnight to form a virion-PEG complex, which was diluted to 10⁴ virions/μL. 100 μL virion-PEG complex was then applied to a gold chip and incubated for 20 min to allow assembly of the virion-oscillators on the chip. The chip was then rinsed with 1×PBS, followed by incubation in 15 nM dithiolalkanearomatic PEG6-COOH overnight to passivate the exposed gold surface. The chip coated with the virion-oscillators was kept hydrated and stored at 4° C. during the modification.

### Detection of Small Molecule Binding to GPCRs Displayed on VirD Oscillators

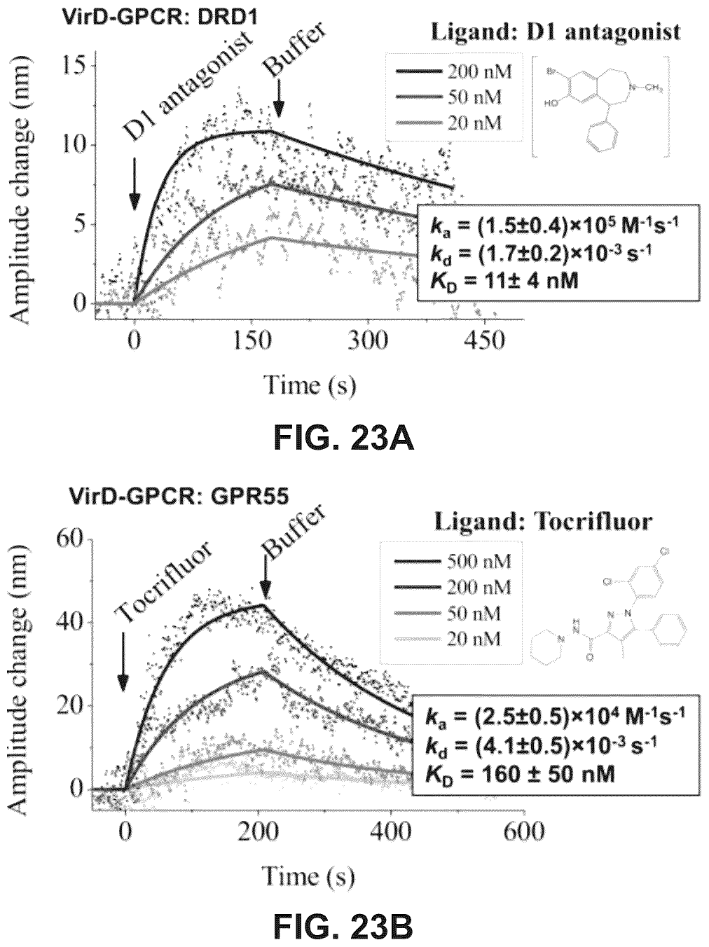

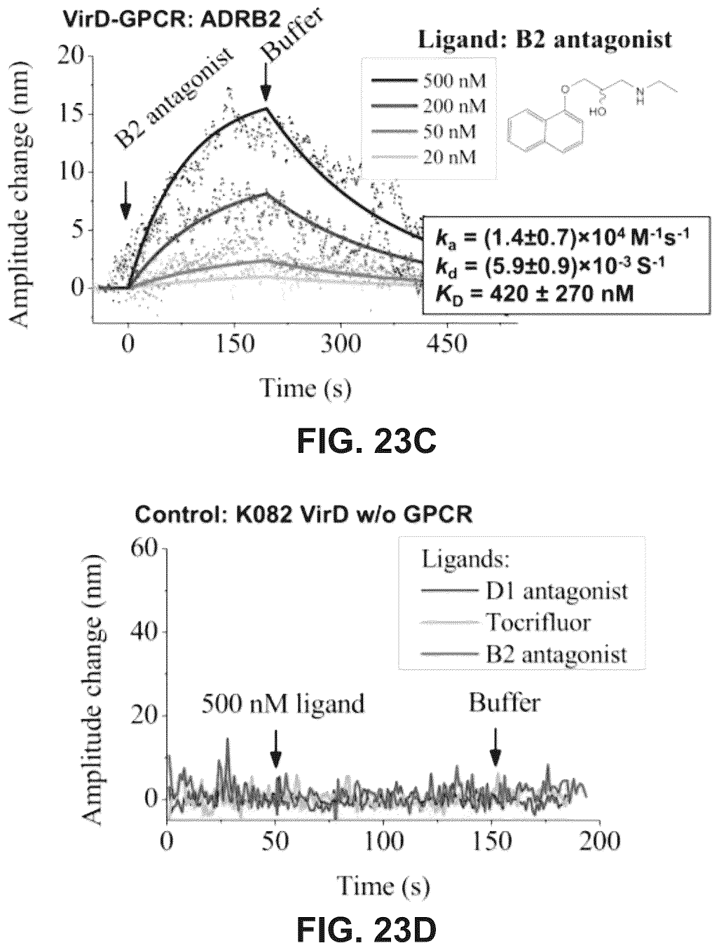

To demonstrate the capability of VirD oscillators, we displayed three human GPCRs (DRD1, GPR55, and ADRB2) on the HSV-1 virion envelope, fabricated virion-oscillators, and measured the binding kinetics of 3 canonical small molecule ligands (D1 antagonist, Tocrifluor, and B2 antagonist) targeting DRD1, GPR55, and ADRB2, respectively (FIG. 23). These GPCRs were chosen because they have well characterized small molecule ligands and are commercially available in both fluorescently labelled and un-labelled forms. To drive the virions into oscillation, we applied a periodic potential with amplitude, 0.4 V, and frequency, 5 Hz, to the gold chip. Initially, we flew 4 mM PBS buffer at a rate of 300 μL/min over the gold chip, and recorded the oscillation amplitude to establish a baseline, and then introduced each of the three ligands to allow binding (association) to the corresponding target GPCR on the virions. The binding induces a change in the oscillation amplitude, indicating changes in the virion effective charge associated with the ligand binding to the GPCRs.

After measuring binding, we studied unbinding of the small molecule ligands from the GPCRs by flowing PBS buffer over the chip and observed that the oscillation amplitude returned to the pre-binding level. By repeating the measurement at different concentrations of each ligand, we obtained binding curves for D1 antagonist-DRD1, Tocrifluor-GPR55, and B2 antagonist-ADRB2 binding pairs (FIGS. 23A-23C). By fitting the binding curves at different concentrations globally with the first order kinetics model, we determined the association rate constant, kₐ, dissociation rate constant k_{d}, and the equilibrium constant K_{D}. Note that at pH 7.4, D1 antagonist and B2 antagonist are positively charged but Tocrifluor is neutral. These results demonstrate that the VirD oscillator can measure binding of both charge and uncharged small molecules, as long as the binding can induce change in the net charge or charge profile.

### Control Experiments

To confirm that the above results were not due to non-specific binding, we used K082, a gB null HSV-1 virion with no GPCR displayed on the envelope, as a negative control and measured its binding with each of the three ligands. We did not observe any detectable changes in the oscillation amplitude (FIG. 23D), indicating no non-specific binding of the ligands to the VirD oscillators. We further cross-validated the binding results with fluorescence imaging of fluorophore-labelled small molecule ligands.

### Fabrication of DNA Barcoded Nano-Oscillators

We fabricated the DNA barcoded nano-oscillators shown in FIG. 18A with the following protocol. Clean glass cover slips pre-coated with 47 nm gold film were immersed overnight in a solution containing biotin-(PEG)n-thiol (10 KDa) and methoxy-(PEG)4-thiol at a ratio of 1:100 to tether the long biotin linker PEG and spacer PEG onto the gold chip. The virons were incubated with biotin-(PEG)n-NHS (1000 Da) at a 1:2 ratio in 1×PBS for 1 hour to form a biotin-(PEG)n modified virion structure. The modified virions were then incubated with streptavidin-coated 150 nm AuNPs at 1:1 ratio in 1×PBS for 30 minutes to obtain the virion-AuNP nanocomplex. To encode the nanocomplexes of different GPCR virions, they were incubated in 20 nM biotin-labeled DNA barcode sequences for 30 minutes, followed by 2× centrifugation at 5000 rpm for 10 min to collect the deposit. Subsequently, the deposit was dispersed in PBS buffer to obtain DNA-coded virion-AuNP nanocomplexes. The one-to-one conjugation of AuNP to the virion was confirmed by scanning electron microscopy as shown in FIG. 18B. Finally, the nanocomplexes were diluted into a concentration about 10⁴/μL and added onto the PEG linker modified gold sensor chip to form a random nano-oscillator array.

### Affinity Encoded Multi-State DNA Barcoding

To demonstrate the feasibility of affinity encoded multi-state DNA barcoding, we fabricated a group of DNA sequences consisting of one decoding oligonucleotide and 3 DNA barcodes with 0, 2 and 3 mismatches to the decoding sequence, as shown in Table 4. Three different types of VirD-GPCRs are labelled with these barcodes, and they are listed in the table along with the corresponding small molecule ligands.

TABLE 4
Proof of concept DNA barcodes and corresponding VirD-GPCRs and
small molecule ligands.

| | | SEQ | | |
|---|---|---|---|---|
| | | ID | VirD- | Small molecule |
| Barcode Name | DNA Sequence | NO: | GPCR | ligands |
| decoding sequence | 5′-GAGAAGGGCCGAGGTATTGT-3′ | 1 | | |
| barcode 1 | biotin-(CH2)6-5′- | 2 | ADRB2 | Propranolol (296 Da) |
| (0 mismatch) | ACAATACCTCGGCCCTTCTC-3′ | | | |
| barcode 2 | biotin-(CH2)6-5′- | 3 | DRD1 | D1 antagonist (332 Da) |
| (2 mismatches) | ACACTACCTCGGCCCTCCTC-3′ | | | |
| barcode 3 | biotin-(CH2)6-5′- | 4 | K082 | N/A |
| (3 mismatches) | ACACTACCTCAGCCCTCCTC-3′ | | | |

Multiplexed Small Molecule Binding Kinetic Measurement with Wide-View Plasmonic Imaging

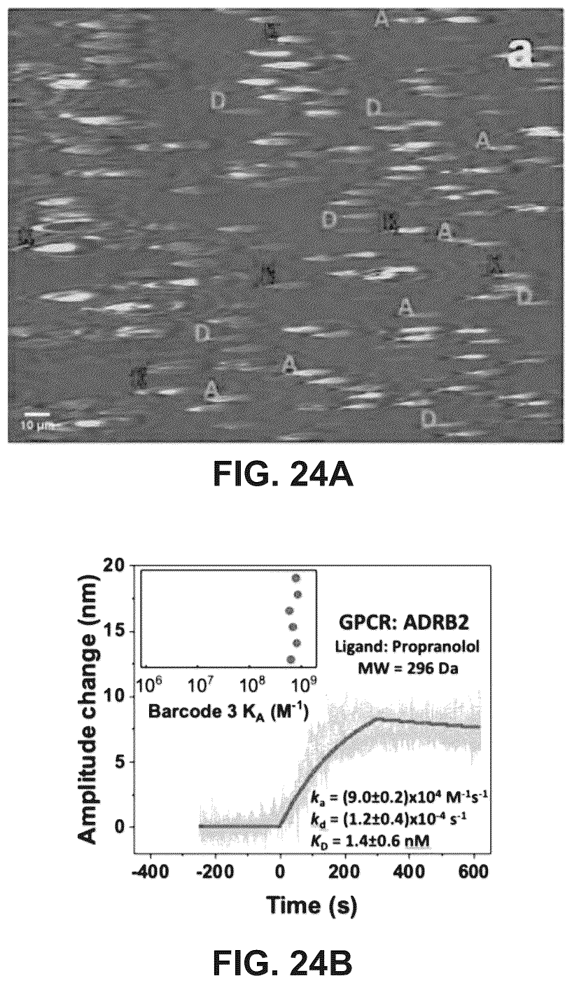

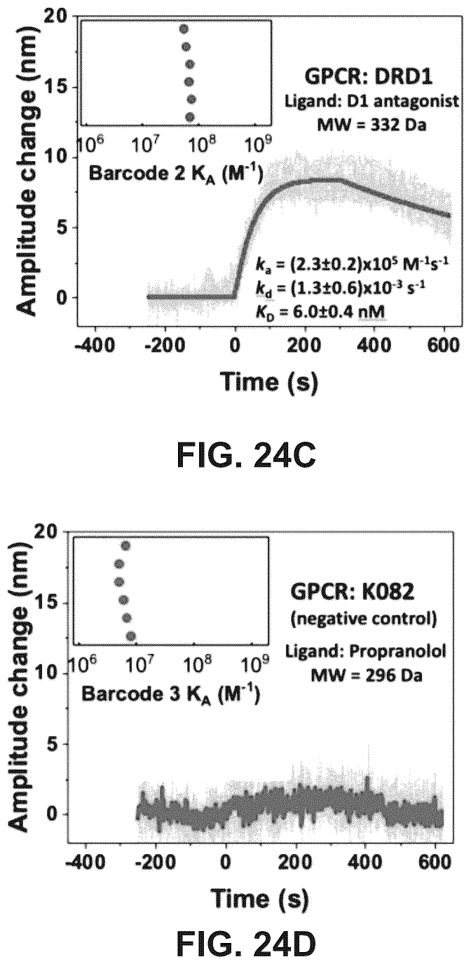

After simultaneous measurement of the binding kinetics of every oscillator on the sensor array, the same plasmonic imaging technique can be used to simultaneously readout the barcodes of every nano-oscillator (FIG. 24A). In preliminary studies, this was achieved with high magnification objectives (e.g., 60×) that have relatively small view size (detection area). However, for high throughput applications, e.g., simultaneous study of 315 human GPCRs on a single chip, the imaging view area must be sufficiently large to encompass thousands of oscillators (in a nanoarray format). This requires wide view plasmonic imaging capability. To meet this requirement, we measured DNA barcoded nanocomposite oscillators in a low magnification (˜10×) prism coupled wide-view plasmonic imaging system for multiplexed detection and decoding using the DNA barcodes and selected GPCRs listed in Table 4. The SFNOA results measured in wide-view are shown in FIG. 24, confirming that one decoding sequence combined with different mismatched barcodes is able to robustly produce distinct hybridization affinities (˜10 fold K_{D}) with narrow distributions sufficient for affinity-base decoding of these barcodes (FIG. 21A). These results highlight the feasibility of our affinity-encoded multi-state DNA barcoding strategy. Additionally, the use of multiple decoding sequences should be able to rapidly expand the number of addressable barcodes, as illustrated in FIG. 20.

### High Spatial Resolution Plasmonic Scattering Imaging for High Density SFNOA.

Redundant measurement on multiple virions of the same binding pair is necessary to ensure high quality measurement and fidelity in the results. Statistically, the minimum number of particles Nₘᵢₙ needed to ensure an effective response in every measurement is:

$${N}_{min} \geq {( \frac{2 S . D .}{Med - Lim} )}^{2} \qquad ( 2 )$$

where Med is the median of the measured responses, Lim is the limit of effective responses, and S.D. is the standard error by counting the measured responses. When the median exceeds the limit of effective response by two-fold of the standard error, then a confidence level of 95% to ensure a significant response is achieved by just detecting Nₘᵢₙ number of particles. For the SFNOA result described herein, Nₘᵢₙ was determined to be 3. Another factor affecting the minimum number of nano-oscillators needed on the chip is the digital noise due to the random nature of the array, which is presented as N=N_{average}±\sqrt{N_{average}}, where N_{average}, is the average number of a certain type of nano-oscillators presented in the plasmonic image. Therefore, both Nₘᵢₙ≥3 and N_{average}≥6 must be satisfied to statistically ensure high quality and fidelity in the results. Consequently, ˜2000 nano-oscillators are required to confidently measure the entire 315 non-odorant human GPCR collection with SFNOA.

With a broad 10× optical zoom, a typical CMOS camera with ⅔ inch sensor size can provide ˜0.5 mm² imaging area, limiting the total number of observable nano-oscillators to ˜1000. This limit is primarily imposed on the array density as a consequence of the inherently large plasmonic propagation length, which generates parabolic patterns from the interference between the individual nanocomplex and the surface plasmon wave. This interference pattern can persist over 20 μm due to the strong scattering effect from the AuNP, as shown in FIG. 24A. Moreover, reducing the optical zoom would be counterproductive, as doing so would reduce sensitivity for measuring the oscillation amplitude.

Consequently, improving the spatial resolution by reducing or removing the interference patterns provides the greatest benefit. We have developed a software-based method to remove the plasmonic tails via image reconstruction, but this approach adds computational cost and processing time to the analysis, and it becomes less effective as the density of particles increases. Hardware-based reduction of the plasmonic tails have also been reported by using azimuthal rotation illumination, however, the method requires complicated hardware to rotate the beam, thus increasing cost, noise, and instability. In this project, we propose to use our recently develop plasmonic scattering imaging technology to eliminate the plasmonic tails and realize high density SFNOA.

### Principle of Plasmonic Scattering Microscopy (PSM):

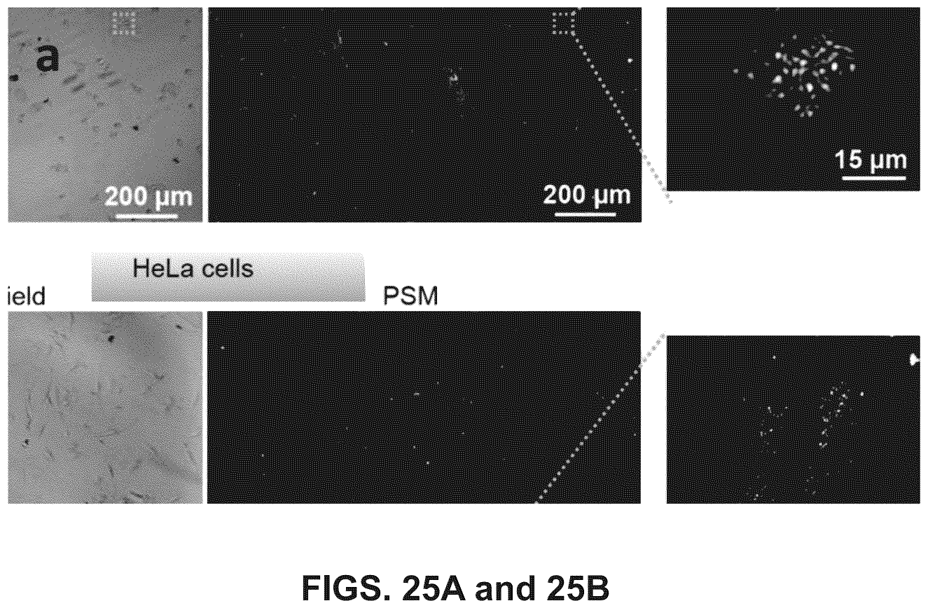

As shown in FIG. 24A, instead of collecting the reflected light as in the conventional plasmonic imaging system, PSM collects the far-field scattered light from the particles on the plasmonic sensor surface. This approach avoids the collection the reflected light that contains the parabolic pattern (FIG. 19A and FIG. 24A) and results in nearly diffraction limited imaging of nanoscale objects (FIG. 25B). Using PSM, we recently successfully imaged single exosomes which are smaller than VirD nanoparticles. PSM uses the same evanescent plasmonic wave as conventional plasmonic imaging to illuminate the sample, therefore retains the same high sensitivity to vertical distance for accurate measurement of the oscillating amplitude of a nano-oscillator.

Prism-Coupled PSM Setup with Electrical Potential Modulation Capability:

We build a prism-coupled PSM setup with a top-mounted long working distance 10× objective as illustrated in FIG. 25A, and integrate potential modulation function to the setup. Briefly, light from a laser diode with center wavelength of 660 nm are collimated to excite the SPR on the gold coated glass slide placed on a prism. The scattered surface plasmonic waves are collected by a 10× microscope objective and imaged by a high resolution USB 3.0 CMOS camera (e.g. FLIR GS3-U3-23S6M-C with up to 163 fps capture rate at 1920×1200 pixels) after a tube lens. A flow cell is used for sample delivery. A pre-patterned double-sided tape gasket is sandwiched between an Indium Tin Oxide (ITO) coated cover glass with two 1 mm drilled holes (as inlet and outlet) and a gold-coated glass slide to form a flow cell. The ITO layer faces down acting as an integrated counter electrode. The gold film acts as the working electrode. Both electrodes are connected to a potentiostat for applying electrical potential modulations to drive the nano-oscillators. A narrow flow cell height of ˜50 μm is set by the thickness of the double-sided tape gasket. Two PDMS blocks with holes created by 0.75 mm disposable biopsy punches are bound to the cover glass after plasma cleaning and baking 1 hour at 90° C. A syringe pump is used to deliver the sample to the flow cell.

### Maximizing Array Density and Imaging Capability:

Nanocomposite oscillators are fabricated according to the protocol described herein with different PEG linker and PEG spacer ratios (e.g. 1:5, 1:10, 1:20, 1:50 and 1:100) and adjusting the nanocomposite concentration if needed, to realize different SFNOA densities. The fabricated oscillators are measured by the PSM setup with electrical modulations at a range of frequencies and voltages. The recorded signals are analyzed to find out the optimal SFNOA density, as well as working potential and frequency. One goal is to reach maximum oscillator density (>2000 nano oscillators in the imaging area) while accurately resolving individual oscillators, and measuring the oscillation amplitudes with <1 nm noise level, corresponding to ˜1.5e charges.

### Multiple Decoding Groups for Multiplexed Decoding of SFNOA.

We construct an additional decoder and corresponding barcodes to establish scalability of the affinity encoded multi-state DNA barcoding scheme for large SFNOA arrays. Our data shows that affinity differences between DNA barcodes with different numbers of mismatched bases to a single decoding sequence can discriminate up to 4 addressable states. To fully address several hundred types of membrane proteins, a decoding scheme with multiple decoding groups is needed.

Two decoding groups of DNA barcodes will be designed and tested to enable up to 16 unique addresses, as shown in FIG. 20. With selected VirD-GPCRs this will demonstrate on a small scale SFNOA's ability to self-assemble multiple DNA barcodes on a single nano-oscillator and to decode the corresponding sequences on single nano-oscillators in a highly parallel fashion. To test feasibility, six DNA barcodes in two groups are tested as listed in Table 5. Each group contains a unique decoding sequence and 3 barcodes with 0, 2 and 3 mismatched bases. Note that no barcode is a valid 4^{th} address. These barcodes are used to label 6 different types of VirD-GPCRs as listed in Table 6 to establish the fabrication protocol. SFNOA arrays are fabricated with these barcode labelled GPCRs. The binding kinetics of the corresponding small molecule ligands are measured, and then serially decoded with the two decoding sequences to establish a protocol for multiplexed multi-state affinity encoded DNA barcoding for large SFNOA arrays. The obtained binding kinetics are validated with individually measured VirD nano-oscillator results and literature-reported affinity (K_{D}) values listed in Table 6.

TABLE 5
Two groups of DNA barcodes.

| | | SEQ |
|---|---|---|
| | | ID |
| Barcode Name | DNA Sequence | NO: |
| Group 1 decoding sequence | 5′-GAGAAGGGCCGAGGTATTGT-3′ | 1 |
| barcode G1-1 (0 mismatch) | biotin-(CH₂)₆-5′- | 2 |
| | ACAATACCTCGGCCCTTCTC-3′ | |
| barcode G1-2 (2 mismatches) | biotin-(CH₂)₆-5′- | 3 |
| | ACACTACCTCGGCCCT&CTC-3′ | |
| barcode G1-3 (3 mismatches) | biotin-(CH₂)₆-5′- | 4 |
| | ACACTACCTCAGCCCTCCTC-3′ | |
| Group 2 decoding sequence | 5′-ACGAAGGTCTGTAGCAACTC-3′ | 5 |
| barcode G2-1 (0 mismatch) | biotin-(CH₂)₆-5′- | 6 |
| | GAGTTGCTACAGACCTTCGT-3′ | |
| barcode G2-2 (2 mismatches) | biotin-(CH₂)₆-5′- | 7 |
| | GAGTTACTACAGACCATCGT-3′ | |
| barcode G2-3 (3 mismatches) | biotin-(CH₂)₆-5′- | 21 |
| | GAGTTACTATAGACCATCGT-3′ | |

TABLE 6
SFNOA with exemplary multiplexed barcodes.

| | | Small Molecule | |
|---|---|---|---|
| Barcodes | GPCR | Ligands | Reported K_{D} (nM) (method) |
| G1-1, G2-1 | NTSR1 | Neurotensin | 17 (fluorescence)⁵³ |
| G1-1, G2-2 | OPRM1 | DynorphinA | 13.8 (Radioligand)⁵⁴ |
| G1-1, G2-3 | ADRB2 | Propranolol | 1.1 (Radioligand)⁵³ |
| G1-2, G2-1 | DRD1 | D1 antagonist | 6.6 (western blotting)⁵⁵ |
| G1-2, G2-2 | SSTR2 | SRIF14 | 4.9 (radioimmunoassay)⁵⁶ |
| G1-3, G2-3 | K082 | N/A | |

Optionally, the conventional plasmonic imaging approach is used along with a CMOS camera with sensor large area, combined with software tail removal to realize detection of ˜2000 oscillators for the 315 non-odorant human GPCRs. If the charged DNA barcode affects the measured binding kinetics of small molecule ligands, this problem can be solved by replacing DNA codes with less charged peptide nucleic acids (PNA).

### High Throughput SFNOA Instrument

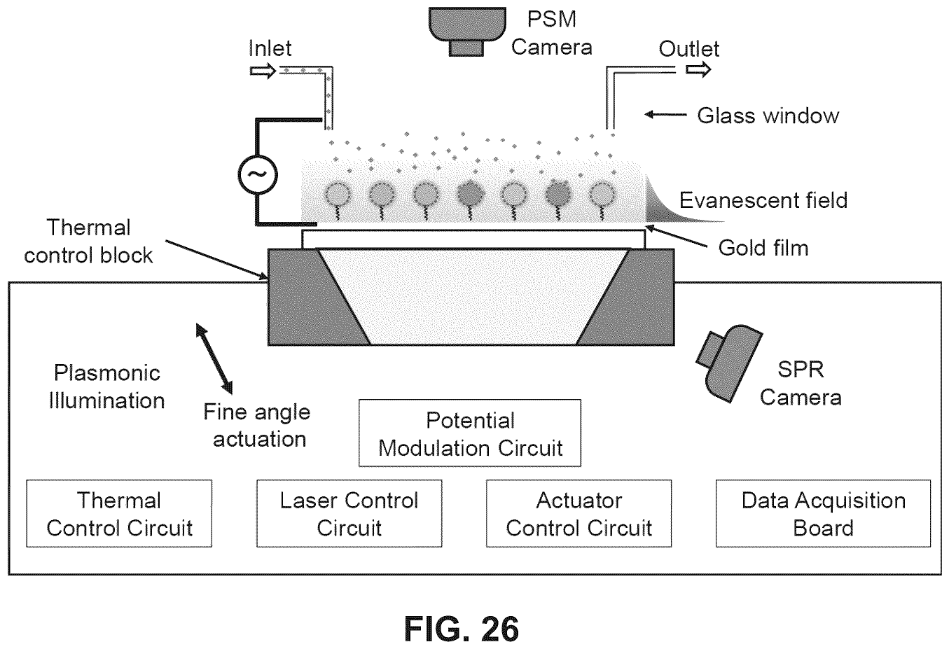

We develop a plasmonic scattering imaging reader for high throughput quantification of molecular binding kinetics of the membrane proteins displayed on the virions. The high throughput SFNOA reader can resolve each individual VirD-Oscillator in high density with a 10× objective and a high-resolution CMOS imager for simultaneous charge-sensitive detection of small molecule binding kinetics on each of the 315 kinds of non-odorant human GPCR oscillators in one measurement (FIG. 26). The system consists of 1) optics for plasmonic scattering detection and imaging, 2) electrical control system to manage controllers and peripheral devices, record data, and drive the SFNOA into oscillation, 3) fluidics for precision sample delivery, and 4) software for data collection and analysis.

Optical imaging system: p-polarized light from a solid-state laser is used to excite surface plasmonic waves on the gold chip placed on top of a prism. A fast high resolution USB 3.0 CMOS imager is used to collect the plasmonic scattering image via a 10× objective mounted on top of the microarray chip.

Electrical circuit (driver of the SFNOA): A custom potential modulation circuit board is built to drive the SFNOA into oscillation. It supports a sine wave potential sweep of 5Vpp up to 1 kHz, which is sufficient to meet project needs. The circuit supports direct connectivity of control electrodes with buffered 1/O lines to prevent damage from high voltage shock. The circuit is connected to a data acquisition board.

Temperature control: Temperature stability is important for accurate and reproducible measurement of binding kinetics. We design a temperature control system to support a range of 10 to 40° C. with stability of ±0.01° C. A precision Peltier element is the mounted to the prism block for efficient heating and cooling of the sensor chip. A PTC series thermal controller from Wavelength Electronics is used to control the temperature.

Sample delivery: Reliable binding kinetics measurements also need a low-noise and reproducible sample delivery. This is achieved with a carefully designed flow cell and a fluidic pump together with an auto-sampler. The flow cell has integrated electrodes for electrically driving the SFNOA. A precision syringe pump with in-line degassing is used to deliver a stable stream of running buffer. The auto-sampler (ALIAS™ from Spark Holland, Netherlands)) is used for fast and reliable testing of multiple solutions.

Data acquisition and analysis software: A data acquisition card from National Instruments Inc. (NI) is used to control digital and analog I/O lines. Data acquisition and analysis software are developed with Matlab and/or C++ for: 1) recording top and bottom plasmonic images along with potential and current; 2) automatically identifying the array spots in the wide-view images, and individual VirD-Oscillators in the high-resolution images; 3) extracting the image intensity of each spot/VirD-Oscillators vs. time; 4) using a Fourier filter to reduce all random noises except at the frequency of oscillation, 5) generating binding curves for each virion; and 6) fitting the data with binding kinetics models.

### Affinity Encoded SFNOA for the Collection of 315 Non-Odorant Human GPCRs

We design and fabricate 5 decoding groups of affinity encoded multi-state DNA barcodes to encoding the entire collection of 315 virion displayed non-odorant human GPCRs. We optimize the SFNOA fabrication protocol and test the storage conditions for both of the fabricated SFNOA chip and the barcoded nanocomposites.

Produce 315 VirD non-odorant human GPCRs: We have developed a GPCR library, including a collection of 315 non-odorant human GPCRs displayed on virions by cloning human GPCRs into the HSV-1 genome using the Gateway method, as described herein. We focus on these non-odorant GPCRs because unlike odorant/sensory GPCRs they play critical roles in signaling pathways involved in various human diseases and are preferred drug targets for treating the diseases. We produce virions displayed with the entire collection of the 315 GPCRs by infected Vero cells following the protocol described herein. Production of the VirD-GPCRs is relatively straight forward, such that the quantity can be readily scaled up as needed. We use these virions to test the nanoarray fabrication protocols and produce SFNOA chips with these non-odorant human GPCRs for further validation of the technology.

Design and fabricate three additional decoding groups (five total): To encode the entire 315 VirD-GPCR collection, 5 decoding groups of DNA barcodes are needed, with each group containing 3 barcodes with different affinities to the decoding sequence. Table 7 lists the design of three additional decoding groups. These barcodes are constructed and tested to confirm that barcodes in each decoding group have distinct affinities.

TABLE 7
Additional 3 groups of DNA barcodes.

| | | SEQ ID |
|---|---|---|
| Barcode Name | DNA Sequence | NO: |
| Group 3 decoding sequence | 5′-TCTGTGATCGTGTAGGATGC-3′ | 9 |
| barcode G3-1 (0 mismatch) | biotin-(CH₂)₆-5′- | 10 |
| | GCATCCTACACGATCACAGA-3′ | |
| barcode G3-2 (2 mismatches) | biotin-(CH₂)₆-5′- | 11 |
| | GCACCCTACACGATCASAGA-3′ | |
| barcode G3-3 (3 mismatches) | biotin-(CH₂)₆-5′- | 12 |
| | GCACCCTACATGATCAAAGA-3′ | |
| Group 4 decoding sequence | 5′-GGTTGTTGACGAGTAGTGTG-3′ | 13 |
| barcode G4-1 (0 mismatch) | biotin-(CH₂)₆-5′- | 14 |
| | CACACTACTCGTCAACAACC-3′ | |
| barcode G4-2 (2 mismatches) | biotin-(CH₂)₆-5′- | 15 |
| | CACATTACTCGTCATCAACC-3′ | |
| barcode G4-3 (3 mismatches) | biotin-(CH₂)₆-5′- | 16 |
| | CACATTACTAGTCATCAACC-3′ | |
| Group 5 decoding sequence | 5′-TGGTGTGTACTTGTGCAGAG-3′ | 17 |
| barcode G5-1 (0 mismatch) | biotin-(CH₂)₆-5′- | 18 |
| | CTCTGCACAAGTACACACCA-3′ | |
| barcode G5-2 (2 mismatches) | biotin-(CH₂)₆-5′- | 19 |
| | CTCTACACAAGTACATACCA-3′ | |
| barcode G5-3 (3 mismatches) | biotin-(CH₂)₆-5′- | 20 |
| | CTCTACACAATTACATACCA-3′ | |

Optimize SFNOA chip fabrication protocol: The protocol described herein is optimized for fabrication of SFNOA at large scale. Key features of the protocol include simplicity and reproducibility with >90% yield and batch-to-batch variability <10%. Key parameters to optimize including: 1) Polymer linker length: PEG with lengths varying from a few tens of nm to a few hundred of nm are available from commercial vendors. A PEG linker with a length of >100 nm will allow the virion to move in and out of the evanescent field completely, maximizing the image contrast change of the virion. However, noise from Brownian motion of the virion increases with the linker length, as the virion is able to move over a larger distance. The dependence of the signal-to-noise ratio on the PEG length are analyzed to determine the length that provides the best results. 2) Optimal AuNP size: The 150 nm AuNP size demonstrated in preliminary tests are the size initially implemented. Larger AuNPs may produce greater plasmonic signal and host more copies of DNA barcodes, but it may also induce non-specific binding of ligands. However, if the AuNP is too large, it will exceed the evanescent field detection range. 200 nm and 500 nm AuNP sizes are tested in case additional surface area for hosting of 5 DNA barcodes is needed. 3) Density of DNA barcodes: The concentration of DNA barcodes are adjusted as needed to determine the optimal barcoding signal while also minimizing interference with sample measurement. 4) Buffer: Buffer may change the effective charge of the virions via charge screening. In our preliminary tests, diluted PBS buffers was primarily used. We examine the effects of buffer type and ionic strength on the performance of the SFNOA.

Test the storage condition of the SFNOA chips and DNA barcoded nanocomposites: The storage condition and shelf life of both the fabricated SFNOA chip and the DNA barcoded nanocomposites containing VirD-GPCRs will be tested. For short-term storage, both the SFNOA gold chip and DNA barcoded nanocomposites can be kept in buffer and stored at 4° C. However, a long-term storage method is needed for product storage and sale. Long-term storage conditions of the chips and the barcoded nanocomposites will be tested, starting with the standard protein microarray storage protocols, such as covered with buffer containing glycerol and store in −80° C. freezer. A six months storage without degradation of the performance can be targeted.

In case not all the barcodes can have 3 distinct affinity states, a mix of 2 and 3 affinity state barcoding groups can be used. For example, if 3 out of the 5 barcode groups can only have j=2 (e.g., none, 0 mismatch and 2 mismatch), we still can have a total of 4²×3³=432 encoding states, sufficient to cover the entire 315 GPCR collection. If long-term storage of pre-printed SFNOA chips is problematic, an easy-to-follow assay kit containing barcoded VirD-nanocomposites for onsite self-fabricate the chips can be developed.

### Test and Validate the Developed SFNOA GPCR Assay

The SFNOA technology is tested by measuring the binding kinetics of small molecule drugs, peptide ligands, and antibodies against human GPCRs and validates the measurements with reference technologies when applicable. The success of these tests validate the SFNOA technology for studying transmembrane protein functions (e.g. orphan GPCR functions), discovering new biomarkers and drug targets, and screening new drugs by ranking the drug candidates based on the measured affinity and kinetic constants.

Ligands identified for non-odorant GPCRs consist of a wide variety of molecules, including small molecules, peptides, and proteins. We test the SFNOA technology by selecting ligands from each class. This allows us to evaluate the capability of the technology for measuring large and small molecule ligands. We initially focus on 10 GPCRs, including those that are relevant to different types of human cancer. For example, ADRB2, BDKR1, CCRL2 and CXCR2 are well-documented for their roles in cancer growth and/or metastasis, and ACKR3, ADRA2A, CCR7, HRH1, KISS1R, and SSTR1 have been identified important for cancer.

Measure binding kinetics of small molecule and peptide ligand binding to GPCRs: We measure the binding kinetics of five small molecule drugs and five peptide ligands with known human GPCR targets (Table 8) following the protocol described earlier. Preliminary tests of these binding pairs are highly promising. These GPCRs are identified because of their importance in cancer, but their binding kinetic constants are difficult to measure with the existing detection technologies. To evaluate the accuracy, we thus only compare the affinity constants measured by the SFNOA technology with the values in the binding database. For binding kinetics, we perform various control experiments, and evaluate the precision of our technology via statistical analysis of 6× replicated measurements.

TABLE 8
Selected small molecule and peptide ligands

| GPCR | Ligand | Class |
|---|---|---|
| ADRB2 | Salmeterol | Small molecule |
| HRH1 | Histamine | Small molecule |
| ADRA2A | Atipamezol | Small molecule |
| CHRM3 | Tiotropium | Small molecule |
| OPRM1 | Alvimopium | Small molecule |
| BDKRB1 | Bradykinin | Peptide |
| NTSR1 | Neurotensin | Peptide |
| KISS1R | Kisspeptin | Peptide |
| SSTR1 | Somatostatin | Peptide |
| TACR1 | Substance P | Peptide |

An additional consideration is that a small molecule may bind to more than one GPCR, a situation reported in literature. However, we expect that the kinetic constants would be noteworthy and different. Surveying the binding of a small molecule with many non-odorant GPCRs in a single assay allows us to rank the binding affinity and kinetics of the small molecules with different GPCRs, which is important for drug screening.

Measure kinetic and affinity constants of antibody binding to GPCRs: Antibody drugs are often designed based on the structures of the canonical ligands, and they may suffer from unwanted side effects associated with off-target binding activities. This is mainly because some GPCRs share high sequence homology in the binding pockets targeted by the drug. Therefore, recent efforts have been devoted to developing antibody-based biologicals targeting ectodomains of the non-odorant GPCRs. The capability of measuring binding kinetics and specificity of anti-GPCR antibodies simultaneously with the SFNOA platform permits observation of these issues early in the development process, thus reducing overall drug development time and cost. We select five commercially available mAbs (Table 9) based on our preliminary studies. Four of them targeting CXCR1/2/5 and CCR7 show ultra-specific binding affinity, while anti-ACKR3 has two off-targets (i.e., CALCR and GPR61) in addition to its intended target. Both cell surface staining and immunoblot analysis confirmed the weaker off-target interactions. It is insightful to compare the differences in binding kinetics between the canonical and off-targets of this mAb. Additional reasons for choosing these mAbs includes: 1) The targeting GPCRs recognize chemokines as their canonical ligands; 2) Some of the GPCRs (e.g., CXCR1 and CXCR2) recognize the same ligand (e.g., IL8); and 3) CXCR1, CXCR2, and CXCR5 share a significant amount of sequence homology. Quantitative binding kinetic study with SFNOA will progress understanding of the structure and function of these GPCRs. We use the half maximal effective concentration (EC50) values obtained from cell-based neutralization tests as initial guidance for the concentration selection (Table 9; data obtained from R&D Systems, USA). We also carry out negative and positive control experiments to validate the results. We compare SFNOA measured binding affinity results with the values obtained from cell-based neutralization test to further validate our technology. We obtain both the kinetic constants and affinity values for the mAbs. We expect that the binding affinity of anti-ACKR3 to the two off-target GPCRs, namely CALCR and GPR61, to be weaker than that of ACKR3. We also compare the kinetic constants for the on- and off-target GPCRs and examine the correlation between the kinetic constants and affinity data. Additionally, we determine the instrument precision by repeated measurements on six different chips for each binding pair listed in Table 9.

TABLE 9
Selected commercial mAbs targeting
ectodomains of human GPCRs

| mAb targets | Catalog No. | Function data |
|---|---|---|
| CCR7 | R&D MAB197 | EC50 = 5 μg/mL |
| CXCR1 | R&D MAB330 | EC50 = 2 μg/mL |
| CXCR2 | R&D MAB331 | EC50 = 5 μg/mL |
| CXCR5 | R&D MAB190 | EC50 = 1.5 μg/mL |
| ACKR3 | R&D MAB4139 | Not available |

Additional exemplary aspects of the present disclosure are provided in the accompanying APPENDICES A and B, which are incorporated by reference in their entireties.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of performing multiplex detection of ligand binding kinetics, the method comprising:
(a) contacting a first ligand with an array of nucleic acid barcoded oscillators disposed on a first surface of a substrate that comprises an electrically conductive coating, wherein the nucleic acid barcoded oscillators each comprise a nanoparticle attached to the first surface via one or more linker moieties, wherein at least a first nucleic acid barcoded oscillator comprises one or more first ligand binding moieties and one or more first barcode coding nucleic acids attached to the nanoparticle of the first nucleic acid barcoded oscillator, wherein at least a second nucleic acid barcoded oscillator comprises one or more second ligand binding moieties and one or more second barcode coding nucleic acids attached to the nanoparticle of the second nucleic acid barcoded oscillator, wherein the first ligand binding moieties differ from the second ligand binding moieties, wherein the first barcode coding nucleic acids differ from the second barcode coding nucleic acids, and wherein the first ligand is contacted with the array of nucleic acid barcoded oscillators under conditions sufficient for the first ligand to at least partially bind to the first and/or second ligand binding moieties;
(b) applying an alternating current electric field to the substrate sufficient to induce the nucleic acid barcoded oscillators to oscillate proximal to the first surface of the substrate;
(c) detecting changes, if any, in oscillation amplitudes of at least the first and second nucleic acid barcoded oscillators over a duration to produce a first set of ligand binding data;
(d) replacing the first ligand from the array of nucleic acid barcoded oscillators with buffer;
(e) repeating steps (a)-(c) using a second ligand that differs from the first ligand to produce a second set of ligand binding data, wherein the second ligand is contacted with the array of nucleic acid barcoded oscillators under conditions sufficient for the second ligand to at least partially bind to the first and/or second ligand binding moieties;
(f) contacting one or more first barcode decoding nucleic acids with the array of nucleic acid barcoded oscillators disposed on the first surface of the substrate, wherein the first barcode decoding nucleic acids are at least partially complementary to the first and/or second barcode coding nucleic acids, and wherein the first barcode decoding nucleic acids are contacted with the array of nucleic acid barcoded oscillators under conditions sufficient for the first barcode decoding nucleic acids to at least partially hybridize with the first and/or second barcode coding nucleic acids;
(g) applying the alternating current electric field to the substrate sufficient to induce the nucleic acid barcoded oscillators to oscillate proximal to the first surface of the substrate;
(h) detecting changes, if any, in oscillation amplitudes of at least the first and second nucleic acid barcoded oscillators over a duration to produce a first set of barcode decoding data;
(i) replacing the first barcode decoding nucleic acids from the array of nucleic acid barcoded oscillators with buffer; detecting changes, if any, in oscillation amplitudes of at least the first and second nucleic acid barcoded oscillators over a duration to produce a first set of ligand binding data;
(j) repeating steps (f)-(h) using one or more second barcode decoding nucleic acids to produce a second set of barcode decoding data, wherein the second barcode decoding nucleic acids are at least partially complementary to the first and/or second barcode coding nucleic acids, wherein the second barcode decoding nucleic acids differ from the first barcode decoding nucleic acids, and wherein the second barcode decoding nucleic acids are contacted with the array of nucleic acid barcoded oscillators under conditions sufficient for the second barcode decoding nucleic acids to at least partially hybridize with the first and/or second barcode coding nucleic acids, thereby performing the multiplex detection of the ligand binding kinetics.

2. The method of claim 1, wherein step (d) comprises washing the first ligand from the array of nucleic acid barcoded oscillators using a buffer.

3. The method of claim 2, comprising detecting changes, if any, in oscillation amplitudes of at least the first and second nucleic acid barcoded oscillators over a duration to produce an additional set of ligand binding data prior performing step (e).

4. The method of claim 1, wherein step (i) comprises washing the first barcode decoding nucleic acids from the array of nucleic acid barcoded oscillators using a buffer.

5. The method of claim 4, comprising detecting changes, if any, in oscillation amplitudes of at least the first and second nucleic acid barcoded oscillators over a duration to produce an additional set of barcode decoding data prior performing step (j).

6. The method of claim 1, comprising using the first and/or second ligand in a pharmaceutical agent development process based at least in part on the first and/or second set of ligand binding data and the first and/or second set of barcode decoding data.

7. The method of claim 1, comprising administering the first and/or second ligand to a subject in need thereof based at least in part on the first and/or second set of ligand binding data and the first and/or second set of barcode decoding data.

8. The method of claim 1, comprising detecting the changes in the oscillation amplitudes of the first and second nucleic acid barcoded oscillators using a plasmonic imaging technique and/or a microscopic imaging technique.

9. The method of claim 1, comprising quantifying the binding kinetics and binding affinity of the first and second ligands using the detected changes in the oscillation amplitudes of the first and second nucleic acid barcoded oscillators over the duration.

10. The method of claim 1, comprising detecting the changes in the oscillation amplitudes of the first and second nucleic acid barcoded oscillators over the duration using a CMOS camera.

11. The method of claim 1, comprising removing the second ligand from the array of nucleic acid barcoded oscillators prior to performing step (f).

12. The method of claim 1, wherein the first surface of the substrate comprises one or more oscillators that lack a barcode coding nucleic acid attached to the nanoparticle.

13. The method of claim 1, wherein one or more of the barcode coding nucleic acids and/or one or more of the barcode decoding nucleic acids comprise a sequence of nucleotides selected from the group consisting of: SEQ ID NOS: 1-20.

14. The method of claim 1, wherein the electrically conductive coating comprises gold (Au), indium tin oxide (ITO), silver (Ag), copper (Cu), and/or aluminum (Al).

15. The method of claim 1, wherein the linker moieties comprise polyethylene glycol (PEG) moieties and/or biomolecules.

16. An oscillator array device, comprising a substrate that comprises a first surface that comprises an electrically conductive coating and an array of nucleic acid barcoded oscillators disposed on the first surface of the substrate, wherein the nucleic acid barcoded oscillators each comprise a nanoparticle attached to the first surface via one or more linker moieties, wherein at least a first nucleic acid barcoded oscillator comprises one or more first ligand binding moieties and one or more first barcode coding nucleic acids attached to the nanoparticle of the first nucleic acid barcoded oscillator, wherein at least a second nucleic acid barcoded oscillator comprises one or more second ligand binding moieties and one or more second barcode coding nucleic acids attached to the nanoparticle of the second nucleic acid barcoded oscillator, wherein the first ligand binding moieties differ from the second ligand binding moieties, and wherein the first barcode coding nucleic acids differ from the second barcode coding nucleic acids.

17. The oscillator array device of claim 16, wherein the first ligand binding moieties comprise at least a first protein that binds, or is capable of binding, to a first and/or a second ligand, wherein the second ligand binding moieties comprise at least a second protein that binds, or is capable of binding, to the first and/or second ligand, wherein the first and second proteins differ from one another, wherein virions are attached to the nanoparticles of the first and the second nucleic acid barcoded oscillators, and wherein viral envelopes of the virions display the first or second proteins.

18. The oscillator array device of claim 17, wherein the virions comprise human herpes simplex virus-1 (HSV-1) virions.

19. The oscillator array device of claim 17, wherein the first and second proteins comprise different G-protein-coupled receptors (GPCRs).

20. A system for performing multiplex detection of ligand binding kinetics, comprising:
a substrate having a first surface and a second surface opposite the first surface, wherein the first surface comprises an electrically conductive coating, wherein an array of nucleic acid barcoded oscillators is disposed on the first surface, wherein the nucleic acid barcoded oscillators each comprise a nanoparticle attached to the first surface via one or more linker moieties, wherein at least a first nucleic acid barcoded oscillator comprises one or more first ligand binding moieties and one or more first barcode coding nucleic acids attached to the nanoparticle of the first nucleic acid barcoded oscillator, wherein at least a second nucleic acid barcoded oscillator comprises one or more second ligand binding moieties and one or more second barcode coding nucleic acids attached to the nanoparticle of the second nucleic acid barcoded oscillator, wherein the first ligand binding moieties differ from the second ligand binding moieties, and wherein the first barcode coding nucleic acids differ from the second barcode coding nucleic acids;
a power source electrically connected to the substrate, which power source is configured to apply an alternating current electric field to the substrate;
an objective lens or a prism disposed proximal to the second surface of the substrate;
a light source configured to introduce light through the objective lens or the prism to induce a plasmonic wave at least proximal to the first surface of the substrate;
a detector configured to collect light reflected from the substrate; and
a controller operably connected at least to the power source, the light source, and the detector, wherein the controller comprises, or is capable of accessing, computer readable media comprising non-transitory computer-executable instructions which, when executed by at least one electronic processor, perform at least:
applying an alternating current electric field to the substrate to induce the nucleic acid barcoded oscillators to oscillate proximal to the first surface of the substrate using the power source;
introducing an incident light toward the second surface of the substrate from the light source to induce the plasmonic wave at least proximal to the first surface of the substrate; and,
detecting changes in oscillation amplitudes of the first and second nucleic acid barcoded oscillators over a duration.
